(19) United States
(12) Patent Application Publication
(10) Pub. No.: US 2005/0032204 A1
(43) Pub. Date: Feb. 10, 2005

(54) Microreactor architecture and methods

(75) Inventors: Seth T. Rodgers, Somerville, MA (US); Andrey J. Zarur, Winchester, MA (US); A. Peter Russo, Woburn, MA (US); Sean J. Leblanc, Westminster, MA (US); Xinyu Li, Woburn, MA (US); Howard B. Schreyer, Malden, MA (US)

Correspondence Address: Timothy J. Oyer, Ph.D.;Wolf, Greenfield & Sacks, P.C., 600 Atlantic Avenue, Boston, MA, 02210, US

(73) Assignee: BioProcessors Corp., Woburn, MA (US)

(21) Appl. No.: 10/664,067

(22) Filed: Sep. 16, 2003

Related U.S. Application Data
(63) Continuation of application No. PCT/US03/25943, filed on Aug. 19, 2003.
(63) Continuation-in-part of application No. 10/456,133, filed on Jun. 5, 2003, now abandoned.
Application No. 10/456,133 is a continuation-in-part of application No. 10/119,917, filed on Apr. 10, 2002.
(60) Provisional application No. 60/409,273, filed on Sep. 9, 2002.
(60) Provisional application No. 60/282,741, filed on Apr. 10, 2001.

(51) Int. Cl. C12M 1/34; C12Q 1/20

(52) U.S. Cl. 435/288.5; 435/305.3; 435/33

(57) ABSTRACT

The present invention generally relates to chemical, biological, and/or biochemical reactor chips and other reaction systems such as microreactor systems, as well as systems and methods for constructing and using such devices. In one aspect, a chip or other reaction system may be constructed so as to promote cell growth within it. In certain embodiments, the chips or other reaction systems of the invention include one or more reaction sites. The reaction sites can be very small, for example, with a volume of less than about 1 ml. In one aspect of the invention, a chip is able to detect, measure and/or control an environmental factor such as the temperature, pressure, CO₂ concentration, O₂ concentration, relative humidity, pH, etc. associated with one or more reaction sites, by using one or more sensors, actuators, processors, and/or control systems. In another aspect, the present invention is directed to materials and systems having humidity and/or gas control, for example, for use with a chip. Such materials may have high oxygen permeability and/or low water vapor permeability. The present invention, in still another aspect, generally relates to light-interacting components suitable for use in chips and other reactor systems. These components may include waveguides, optical fibers, light sources, photodetectors, optical elements, and the like.

### RELATED APPLICATIONS

This application is a continuation of International Patent Application No. PCT/US03/25943, filed Aug. 19, 2003, entitled “Microreactor Architecture and Methods,” by Rodgers, et al., which application claims priority to U.S. Provisional Patent Application Ser. No. 60/409,273, filed Sept. 9, 2002, entitled “Protein Production and Screening Methods,” by Zarur, et al.; U.S. patent application Ser. No. 10/223,562, filed Aug. 19, 2002, entitled “Fluidic Device and Cell-Based Screening Method,” by Schreyer, et al.; U.S. patent application Ser. No. 10/457,048, filed Jun. 5, 2003, entitled “Reactor Systems Responsive to Internal Conditions,” by Miller, et al.; U.S. patent application Ser. No. 10/456,934, filed Jun. 5, 2003, entitled “Systems and Methods for Control of Reactor Environments,” by Miller, et al.; U.S. patent application Ser. No. 10/456,133, filed Jun. 5, 2003, entitled “Microreactor Systems and Methods,” by Rodgers, et al.; U.S. patent application Ser. No. 10/457,049, filed Jun. 5, 2003, entitled “Materials and Reactor Systems having Humidity and Gas Control,” by Rodgers, et al.; and U.S. patent application Ser. No. 10/457,015, filed Jun. 5, 2003, entitled “Reactor Systems Having a Light-Interacting Component,” by Miller, et al. This application is also a continuation-in-part of said U.S. patent application Ser. No. 10/456,133, which application is a continuation-in-part of U.S. patent application Ser. No. 10/119,917, filed Apr. 10, 2002, entitled “Microfermentor Device and Cell Based Screening,” by Zarur, et al., which application claims priority to U.S. Provisional Patent Application Ser. No. 60/282,741, filed Apr. 10, 2001, entitled “Microfermentor Device and Cell Based Screening,” by Zarur, et al. This application is also a continuation-in-part of said U.S. patent application Ser. No. 10/457,049, which application claims priority to U.S. Provisional Patent Application Ser. No. 60/386,323, filed Jun. 5, 2002, entitled “Materials and Reactors having Humidity and Gas Control,” by Rodgers, et al. This application is also a continuation-in-part of said U.S. patent application 10/457,015, which application claims priority to U.S. Provisional Patent Application Ser. No. 60/386,322, filed Jun. 5, 2002, entitled “Reactor Having Light-Interacting Component,” by Miller, et al. All of these applications are incorporated herein by reference.

### BACKGROUND

1. Field of the Invention

The present invention generally relates to chemical, biological, and/or biochemical reactor chips and other reaction systems such as microreactor systems.

2. Description of the Related Art

A wide variety of reaction systems are known for the production of products of chemical and/or biochemical reactions. Chemical plants involving catalysis, biochemical fermenters, pharmaceutical production plants, and a host of other systems are well-known. Biochemical processing may involve the use of a live microorganism (e.g., cells) to produce a substance of interest.

Cells are cultured for a variety of reasons. Increasingly, cells are cultured for proteins or other valuable materials they produce. Many cells require specific conditions, such as a controlled environment. The presence of nutrients, metabolic gases such as oxygen and/or carbon dioxide, humidity, as well as other factors such as temperature, may affect cell growth. Cells require time to grow, during which favorable conditions must be maintained. In some cases, such as with particular bacterial cells, a successful cell culture may be performed in as little as 24 hours. In other cases, such as with particular mammalian cells, a successful culture may require about 30 days or more.

Typically, cell cultures are performed in media suitable for cell growth and containing necessary nutrients. The cells are generally cultured in a location, such as an incubator, where the environmental conditions can be controlled. Incubators traditionally range in size from small incubators (e.g., about 1 cubic foot) for a few cultures up to an entire room or rooms where the desired environmental conditions can be carefully maintained.

Recently, as described in International Patent Application Ser. No. PCT/US01/07679, published on Sept. 20, 2001 as WO 01/68257, entitled “Microreactors,” incorporated herein by reference, cells have also been cultured on a very small scale (i.e., on the order of a few milliliters or less), so that, among other things, many cultures can be performed in parallel.

### SUMMARY OF THE INVENTION

The present invention generally relates to chemical, biological, and/or biochemical reactor chips and other reaction systems such as microreactor systems. The subject matter of this invention involves, in some cases, interrelated products, alternative solutions to a particular problem, and/or a plurality of different uses of one or more systems and/or articles.

In one aspect, the invention is an apparatus. The apparatus, in one set of embodiments, includes a chip comprising a predetermined reaction site having a volume of less than about 1 ml. In one embodiment, the apparatus also includes an active control system able to control an environmental factor associated with the chip in response to a signal indicative of a condition associated with the chip, so as to support a living cell within the predetermined reaction site. The apparatus, in another embodiment, includes a control system able to control an environmental factor associated with the predetermined reaction site, the environmental factor being at least one of relative humidity, pH, dissolved O₂ concentration, dissolved CO₂ concentration, and concentration of a media component.

According to another embodiment, the apparatus may include a control system able to produce a change in a first environmental factor associated with the predetermined reaction site within 1 s of and responsive to a change in a second environmental factor associated with the predetermined reaction site. In still another embodiment, the apparatus may include an active control system able to control an environment within the predetermined reaction site so as to support a living cell for a period of at least 1 day. In yet another embodiment, the apparatus includes a membrane substantially transparent to incident electromagnetic radiation in the infrared to ultraviolet range having a pore size less than 2.0 microns in fluid communication with the predetermined reaction site.

According to another embodiment, the apparatus also includes a component separating the predetermined reaction site from a source of a non-pH-neutral composition. In still another embodiment, the apparatus can include a precursor able to react to form a gaseous agent able to substantially alter the pH of a substance within the predetermined reaction site, where the chip is arranged to allow gaseous non-liquid transport of the agent to the predetermined reaction site. In yet another embodiment, the apparatus includes a pH-altering agent dispensing unit integrally connected to the chip in fluid communication with the predetermined reaction site. The invention, in accordance with another embodiment, includes a source of gas integrally connected to the chip. In another embodiment, the invention includes a laser waveguide in optical communication with a surface defining the predetermined reaction site.

In yet another embodiment, the apparatus includes a sensor integrally connected to the chip, where the sensor is able to determine an environmental factor associated with the predetermined reaction site. The environmental factor is at least one of pH, a concentration of a dissolved gas, molarity, osmolarity, glucose concentration, glutamine concentration, pyruvate concentration, apatite concentration, color, turbidity, viscosity, a concentration of an amino acid, a concentration of a vitamin, a concentration of a hormone, serum concentration, a concentration of an ion, shear rate, and degree of agitation. In some cases, the apparatus may also include an actuator integrally connected to the chip, where the actuator is able to alter the environmental factor.

In another embodiment, the apparatus includes a first sensor integrally connected to the chip, the first sensor able to determine at least one of temperature and pressure, and a second sensor, integrally connected to the chip, that is able to determine a second environmental factor. The second environmental factor, in certain cases, is at least one of pH, a concentration of a dissolved gas, molarity, osmolarity, glucose concentration, glutamine concentration, pyruvate concentration, apatite concentration, color, turbidity, viscosity, a concentration of an amino acid, a concentration of a vitamin, a concentration of a hormone, serum concentration, a concentration of an ion, shear rate, and degree of agitation. In some cases, the apparatus may also include an actuator integrally connected to the chip able to alter at least one of the temperature, the pressure, and the environmental factor.

The apparatus, according to another embodiment of the invention, may include a sensor able to determine an environmental factor associated with at least one of the predetermined reaction sites. The environmental factor may be at least one of the CO₂ concentration, glucose concentration, glutamine concentration, pyruvate concentration, apatite concentration, serum concentration, a concentration of a vitamin, a concentration of an amino acid, and a concentration of a hormone.

In another set of embodiments, the apparatus includes a chip comprising a predetermined reaction site having an inlet, an outlet, and a volume of less than about 1 ml. The predetermined reaction site constructed and arranged to maintain at least one living cell at the predetermined reaction site. In some cases, the chip is constructed and arranged to stably connect in a predetermined, aligned relationship to other, similar chips.

In one set of embodiments, the apparatus includes a chip comprising a predetermined reaction site having an inlet, an outlet, and a volume of less than about 1 ml, where the chip is constructed and arranged to be stably connectable to a microplate. The apparatus, in accordance with another set of embodiments, includes a chip comprising a predetermined reaction site having an inlet, an outlet, and a volume of less than about 1 ml, where the chip is constructed and arranged to be fluid communicable with an apparatus constructed and arranged to address a well of a microplate. In yet another set of embodiments, the apparatus includes a chip comprising a predetermined reaction site having an inlet, an outlet, and a volume of less than about 1 ml, where each predetermined reaction site overlaps at least one well of a microplate. The apparatus, in still another set of embodiments, includes a substantially liquid-tight chip comprising a predetermined reaction site having a volume of less than about 1 ml, where the predetermined reaction site is constructed and arranged to maintain at least one living cell at the predetermined reaction site.

The apparatus, in one set of embodiments, is defined, at least in part, by a chip produced by a process including the step of fastening two components to produce a portion of the chip defining a predetermined reaction site having a volume of less than about 1 ml, where the predetermined reaction site is constructed and arranged to maintain at least one living cell at the predetermined reaction site. The apparatus, in another set of embodiments, includes a chip comprising a predetermined reaction site having a volume of less than about 1 ml, where the predetermined reaction site constructed and arranged to maintain at least one living cell at the predetermined reaction site, and the predetermined reaction site has a nonzero evaporation rate of less than about 100 microliters/day.

According to another set of embodiments, the apparatus includes a predetermined reaction site having a volume of less than about 1 ml, that is constructed and arranged to carry out a chemical or biological reaction promoted by or monitored by electromagnetic radiation within a predetermined wavelength range, and a membrane, transparent to electromagnetic radiation within the predetermined wavelength range to the extent necessary to promote or monitor the reaction, having a pore size of less than 2.0 microns in fluid communication with the predetermined reaction site.

In accordance with another set of embodiments, the apparatus is defined, at least in part, by a chip comprising a first predetermined reaction site having a volume of less than about 1 ml and a second predetermined reaction site, where the chip defines a pathway fluidly connecting the first predetermined reaction site and the second predetermined reaction site, and where the pathway crosses a membrane.

The apparatus, in one set of embodiment, includes a reaction site having a first portion and a second portion separated by a membrane, and at least a first and a second channel in fluidic communication with the second portion of the reaction site.

The invention is a method in another aspect. The method, in one set of embodiments, includes an act of permeating a pH-altering agent into a predetermined reaction site having a volume of less than about 1 ml. According to another set of embodiments, the method includes at least acts of providing a chip comprising a predetermined reaction site having a volume of less than about 1 ml, generating an acid or a base proximate the predetermined reaction site, and contacting the acid or base with a substance within the predetermined reaction site to substantially alter the pH of the substance. In another set of embodiments, the method includes providing a chip defining at least one compartment, the chip further comprising a predetermined reaction site having a volume of less than about 1 ml, and permeabilizing a component positioned between the predetermined reaction site and the compartment.

In accordance with one set of embodiments, the method includes producing a gas in a chip comprising a predetermined reaction site having a volume of less than about 1 ml by directing a laser at at least a portion of the chip.

According to one set of embodiments, the invention, in a method of producing a chip comprising a predetermined reaction site having a volume of less than 1 ml, includes attaching a first component of the chip to a second component of the chip with or without auxiliary adhesive to produce a portion of the chip that defines the predetermined reaction site.

The method, in yet another set of embodiments, includes an act of providing a substrate having a surface into which is fabricated a plurality of reaction sites, where at least one reaction site has a volume less than about 2 ml and is divided by a substantially cell impermeable membrane into at least a cell culture portion containing cells and a reservoir portion not containing cells, where the reservoir portion is fluidly connected to at least a first and a second channel fabricated into the surface of the substrate. The method also includes acts of introducing at least one test compound into at least one of the plurality of reaction sites, and monitoring the effect of the test compound on cells located within the cell culture portion.

In another aspect, the present invention is directed to a method of making one or more of the embodiments described herein, for example, a chip or other reaction system, such as a microreactor system. In yet another aspect, the present invention is directed to a method of using one or more of the embodiments described herein, for example, a chip or other reaction system, such as a microreactor system. In still another aspect, the present invention is directed to a method of promoting one or more of the embodiments described herein, for example, a chip or other reaction system, such as a microreactor system.

In another aspect, the present invention is directed to a method of making a chip and/or a reactor system, e.g., as described in any of the embodiments herein. In yet another aspect, the present invention is directed to a method of using a chip and/or a reactor system, e.g., as described in any of the embodiments herein, for example, example. In still another aspect, the present invention is directed to a method of promoting a chip and/or a reactor system, e.g., as described in any of the embodiments herein.

Other advantages and novel features of the invention will become apparent from the following detailed description of the various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For the purposes of clarity, not every component is labeled in every figure, nor is every component of each embodiment of the invention shown where illustration is not necessary to allow those of ordinary skill in the art to understand the invention. In the figures:

FIG. 1 illustrates one embodiment of the invention;

FIG. 2 illustrates an example of a microfluidic chip for use with the invention including mixing, heating/dispersion, reaction, and separation units, in expanded view;

FIGS. 3A-3C illustrate various stackable arrangements of chips of the invention;

FIGS. 4A-4C illustrate various energy directors for use with the invention in certain embodiments;

FIGS. 5A and 5B illustrate a device according to one embodiment of the invention, having multiple layers;

FIG. 6 is a block diagram of an example of a control system of the invention;

FIGS. 7A and 7B illustrate a device according to another embodiment of the invention having a dispensing unit;

FIGS. 8A and 8B illustrate a device according to another embodiment of the invention where a laser is used to produce a response;

FIGS. 9A and 9B are cross sectional views of certain embodiments of the present invention;

FIGS. 10A-10D illustrates certain membranes of the invention in fluid communication with various reaction sites.

FIG. 11 is an illustration of the dependence of oxygen permeance on film thickness in one embodiment of the invention;

FIG. 12 is a plot of oxygen transmission versus water vapor transmission for various membranes, including certain membranes used in the invention;

FIG. 13 is a graph of pH versus relative intensity, in accordance with one embodiment of the invention;

FIG. 14 is a graph of optical density versus time, demonstrating control of an environmental factor according to an embodiment of the invention;

FIG. 15 illustrates one embodiment of the invention, showing a light interaction with a reaction site;

FIG. 16 illustrates the change of a pH indicator with respect to time in an embodiment of the invention;

FIGS. 17A and 17B (expanded) illustrate portions of various chips according to one embodiment of the invention;

FIGS. 18A and 18B illustrate expanded views of portions of various chips according to another embodiment of the invention;

FIG. 19 illustrates an expanded view of a portion of a chip according to yet another embodiment of the invention;

FIG. 20 is a graph illustrating oxygen permeability for an embodiment of the invention as used in a bacterial culture;

FIG. 21 is a graph illustrating oxygen permeability for an embodiment of the invention as used in a mammalian cell culture;

FIG. 22 illustrates another embodiment of the invention having a waveguide;

FIG. 23 is a graph of intensity (in relative units) versus relative concentration, in an embodiment of the invention;

FIG. 24 is a graph of optical density at 480 nm versus time in an experiment using an embodiment of the invention;

FIG. 25 illustrates a solid substrate having a reaction site and channels, in accordance with one embodiment of the invention;

FIGS. 26A-26E illustrate various views of the embodiment illustrated in FIG. 25; and

FIGS. 27A and 27B illustrate microfabricated bioreactors in accordance with various embodiments of the invention.

### DETAILED DESCRIPTION

The following applications are incorporated herein by reference: U.S. Provisional Patent Application Ser. No. 60/282,741, filed Apr. 10, 2001, entitled “Microfermentor Device and Cell Based Screening Method,” by Zarur, et al.; U.S. patent application Ser. No. 10/119,917, filed Apr. 10, 2002, entitled “Microfermentor Device and Cell Based Screening Method,” by Zarur, et al.; International Patent Application No. PCT/US02/11422, filed Apr. 10, 2002, entitled “Microfermentor Device and Cell Based Screening Method,” by Zarur, et al.; U.S. Provisional Patent Application Ser. No. 60/386,323, filed Jun. 5, 2002, entitled “Materials and Reactors having Humidity and Gas Control,” by Rodgers, et al.; U.S. Provisional Patent Application Ser. No. 60/386,322, filed Jun. 5, 2002, entitled “Reactor Having Light-Interacting Component,” by Miller, et al.; U.S. patent application Ser. No. 10/223,562, filed Aug. 19, 2002, entitled “Fluidic Device and Cell-Based Screening Method,” by Schreyer, et al.; U.S. Provisional Patent Application Ser. No. 60/409,273, filed Sep. 24, 2002, entitled “Protein Production and Screening Methods,” by Zarur, et al.; U.S. patent application Ser. No. 10/457,048, filed Jun. 5, 2003, entitled “Reactor Systems Responsive to Internal Conditions,” by Miller, et al.; U.S. patent application Ser. No. 10/456,934, filed Jun. 5, 2003, entitled “Systems and Methods for Control of Reactor Environments,” by Miller, et aL.; U.S. patent application Ser. No. 10/456,133, filed Jun. 5, 2003, entitled “Microreactor Systems and Methods,” by Rodgers, et al.; U.S. patent application Ser. No. 10/457,049, filed Jun. 5, 2003, entitled “Materials and Reactor Systems having Humidity and Gas Control,” by Rodgers, et al.; an International Patent Application, filed Jun. 5, 2003, entitled “Materials and Reactor Systems having Humidity and Gas Control,” by Rodgers, et aL.; U.S. patent application Ser. No. 10/457,015, filed Jun. 5, 2003, entitled “Reactor Systems Having a Light-Interacting Component,” by Miller, et al.; an International Patent Application, filed Jun. 5, 2003, entitled “Reactor Systems Having a Light-Interacting Component,” by Miller, et aL.; U.S. patent application Ser. No. 10/457,017, filed Jun. 5, 2003, entitled “System and Method for Process Automation,” by Rodgers, et al.; and U.S. patent application Ser. No. 10/456,929, filed Jun. 5, 2003, entitled “Apparatus and Method for Manipulating Substrates,” by Zarur, et al.

The present invention generally relates to chemical, biological, and/or biochemical reactor chips and other reaction systems such as microreactor systems, as well as systems and methods for constructing and using such devices. In one aspect, a chip or other reaction system may be constructed so as to promote cell growth within it. In certain embodiments, the chips or other reaction systems of the invention include one or more reaction sites. The reaction sites can be very small, for example, with a volume of less than about 1 ml. In one aspect of the invention, a chip is able to detect, measure and/or control an environmental factor such as the temperature, pressure, CO₂ concentration, O₂ concentration, relative humidity, pH, etc. associated with one or more reaction sites, by using one or more sensors, actuators, processors, and/or control systems. In another aspect, the present invention is directed to materials and systems having humidity and/or gas control, for example, for use with a chip. Such materials may have high oxygen permeability and/or low water vapor permeability. The present invention, in still another aspect, generally relates to light-interacting components suitable for use in chips and other reactor systems. These components may include waveguides, optical fibers, light sources, photodetectors, optical elements, and the like.

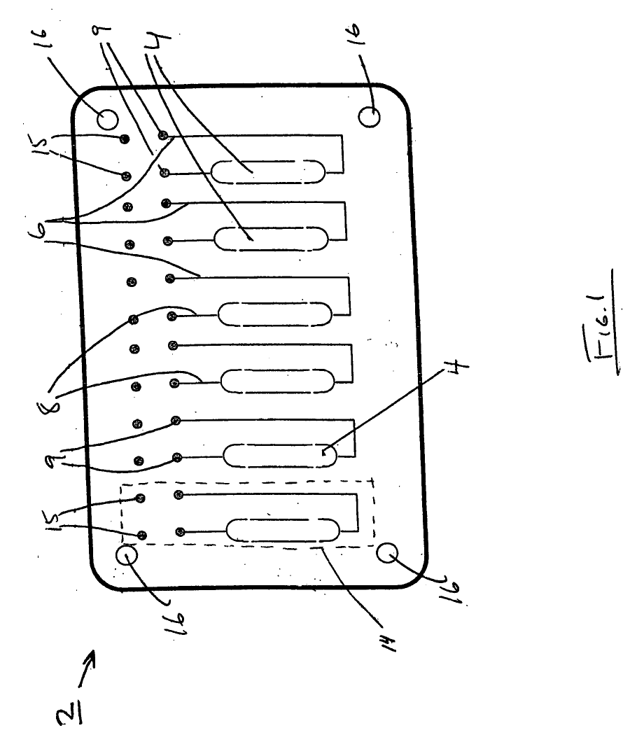

Referring now to FIG. 1, one portion of a chip according to one embodiment is illustrated schematically. The portion illustrated is a layer 2 which includes within it a series of void spaces which, when layer 2 is positioned between two layers (a top and bottom layer relative to the plane of FIG. 1, not shown) define a series of enclosed channels and reaction sites. The overall arrangement into which layer 2 can be assembled to form a chip will be understood more clearly from the description below with respect to other figures.

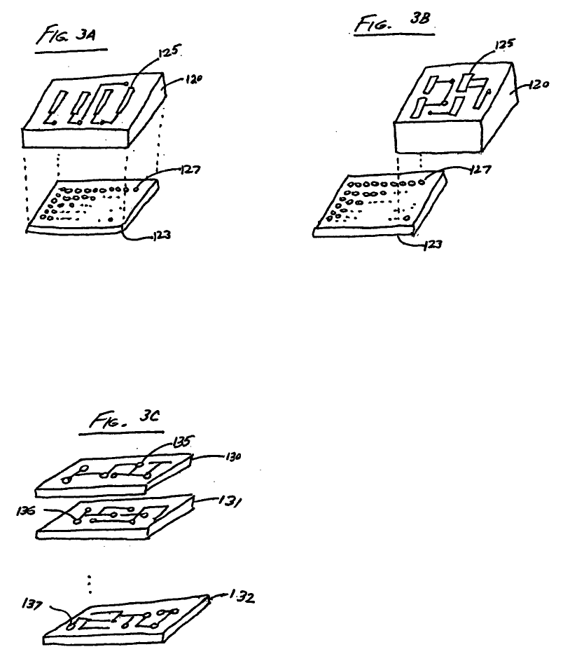

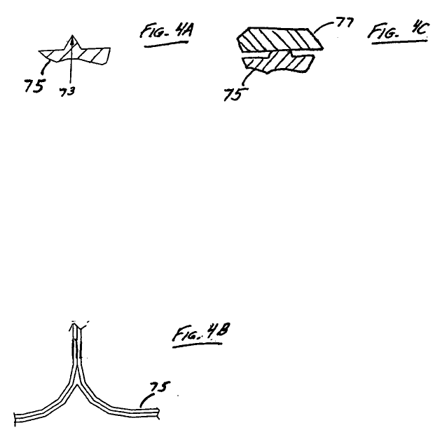

FIG. 1 represents an embodiment including six reaction sites 4 (analogous to, for example, reaction site 125 of FIG. 3A, or reaction site 112 of FIG. 5A, described below). Reaction sites 4 define a series of generally aligned, elongated, rounded rectangular voids within a relatively thin, generally planar piece of material defining layer 2. Reaction sites 4 can be addressed by a series of channels including channels 6 for delivering species to reaction sites 4 and channels 8 for removal of species from the reaction sites. Of course, any combination of channels can be used to deliver and/or remove species from the reaction sites. For example, channels 8 can be used to deliver species to the reaction sites while channels 6 can be used to remove species, etc. Although shown as lines in FIG. 1, channels 6 and 8 are to be understood to define voids within layer 2 which, when covered above and/or below by other layers, may become enclosed channels. Each of channels 6 and 8, in the embodiment illustrated in FIG. 1, is addressed by a port 9. Where port 9 is connected to an inlet channel it can define an inlet port, and where fluidly connected to an outlet channel it can define an outlet port. In the embodiment illustrated, port 9 is a void that is larger in width than the width of channels 6 or 8. Those of ordinary skill in the art will recognize a variety of techniques for accessing ports 9 and utilizing them to introduce species into channels, and/or remove species from channels addressed by those ports. As one example, port 9 can be a “self-sealing” port, addressable by a needle (as described more fully below) when at least one side of port 9 is covered by a layer (not shown) of material which, when a needle is inserted through the material and withdrawn, forms a seal generally impermeable to species such as fluids introduced into or removed from the chip via the port.

Also shown in FIG. 1 are a series of ports 15, not shown to be fluidly connected or connectable to any inlet channels, outlet channels, or reaction sites of the chip. Ports 15 can be defined by voids in layer 2, and can be used to facilitate fluidic connection between and among various layers of a chip and/or an environment external to the chip. As an example, where layer 2 forms part of a multi-layer chip including multiple reaction sites in different layers, another layer may be provided on one side of layer 2 (optionally separated by an intermediate layer or layers) including one set of reaction sites or conduits, and another layer may be provided on the opposite side of layer 2, similarly separated by intermediate layers if desirable, and ports 15 may define passages or routes for fluidic connection between reaction sites and/or conduits of chip layers on opposite sides of layer 2. Ports 15 also may connect to channels communicating with a chamber aligned with a chamber defining reaction site 4, separated from the reaction site by a membrane, e.g. semipermeable membrane. In this way, fluid can be independently flowed into, out of, and/or through a space on one side of a membrane, and also independently through a space on the other side of the membrane, one or both defining a chamber and/or reaction site.

In FIG. 1, each reaction site 4, along with the associated fluidic connections (e.g., channels 6 and 8, ports 9 and ports 15), together define a reactor 14, as indicated by dotted lines. In FIG. 1, layer 2 contains six such reactors, each reactor having substantially the same configuration. In other embodiments, a reactor may include more than one reaction site, channels, ports, etc. Additionally, a chip layer may have reactors that do not substantially have the same configuration.

Additionally shown in FIG. 1 is a series of devices 16 which can be used to secure layer 2 to other layers of a chip and/or to assure alignment of layer 2 with other layers and/or other systems to which the chip is desirably coupled. Devices 16 can define screws, posts, indentations (i.e., that match corresponding protrusions of other layers or devices), or the like. Those of ordinary skill in the art are aware of a variety of suitable techniques for securing layers to other layers and/or chips of the invention to other components or systems using devices such as these.

A variety of definitions are now provided which will aid in understanding of the invention. Following, and interspersed with these definitions, is further disclosure, including descriptions of figures, that will fully describe the invention. Components shown in the figures that follow can generally be used in conjunction with layer 2 of FIG. 1. It is to be understood that in FIG. 1, and in all of the other figures, the arrangement of reaction sites, number of reaction sites, arrangement of channels addressing reaction sites, ports, and the like are merely given as examples that fall within the overall invention.

The term “determining,” as used herein, generally refers to the measurement and/or analysis of a substance (e.g., within a reaction site), for example, quantitatively or qualitatively, or the detection of the presence or absence of the substance. “Determining” may also refer to the measurement and/or analysis of an interaction between two or more substances, for example, quantitatively or qualitatively, or by detecting the presence or absence of the interaction. Examples of techniques suitable for use in the invention include, but are not limited to, gravimetric analysis, calorimetry, pressure or temperature measurement, spectroscopy such as infrared, absorption, fluorescence, UV/visible, FTIR (“Fourier Transform Infrared Spectroscopy”), or Raman; gravimetric techniques; ellipsometry; piezoelectric measurements; immunoassays; electrochemical measurements; optical measurements such as optical density measurements; circular dichroism; light scattering measurements such as quasielectric light scattering; polarimetry; refractometry; or turbidity measurements, including nephelometry.

A “chip,” as used herein, is an integral article that includes one or more reactors. “Integral article” means a single piece of material, or assembly of components integrally connected with each other. As used herein, the term “integrally connected,” when referring to two or more objects, means objects that do not become separated from each other during the course of normal use, e.g., cannot be separated manually; separation requires at least the use of tools, and/or by causing damage to at least one of the components, for example, by breaking, peeling, etc. (separating components fastened together via adhesives, tools, etc.).

A chip can be connected to or inserted into a larger framework defining an overall reaction system, for example, a high-throughput system. The system can be defined primarily by other chips, chassis, cartridges, cassettes, and/or by a larger machine or set of conduits or channels, sources of reactants, cell types, and/or nutrients, inlets, outlets, sensors, actuators, and/or controllers. Typically, the chip can be a generally flat or planar article (i.e., having one dimension that is relatively small compared to the other dimensions); however, in some cases, the chip can be a non-planar article, for example, the chip may have a cubical shape, a curved surface, a solid or block shape, etc.

As used herein, a “membrane” is a three-dimensional material having any shape such that one of the dimensions is substantially smaller than the other dimensions. In some cases, the membrane may be generally flexible or non-rigid. As an example, a membrane may be a rectangular or circular material with a length and width on the order of millimeters, centimeters, or more, and a thickness of less than a millimeter, and in some cases, less than 100 microns, less than 10 microns, or less than 1 micron or less. The membrane may define a portion of a reaction site and/or a reactor, or the membrane may be used to divide a reaction site into two or more portions, which may have volumes or dimensions which are substantially the same or different. Some membranes may be semipermeable membranes, which those of ordinary skill in the art will recognize to be membranes permeable with respect to at least one species, but not readily permeable with respect to at least one other species. For example, a semipermeable membrane may allow oxygen to permeate across it, but not allow water vapor to do so, or allows water vapor to permeate it, but at a permeability that is at least an order of magnitude less. Or a semipermeable membrane may be selected to allow water to permeate across it, but not certain ions. For example, the membrane may be permeable to cations and substantially impermeable to anions, or permeable to anions and substantially impermeable to cations (e.g., cation exchange membranes and anion exchange membranes). As another example, the membrane may be substantially impermeable to molecules having a molecular weight greater than about 1 kilodalton, 10 kilodaltons, or 100 kilodaltons or more. In one embodiment, the membrane may be impermeable to cells, but be chosen to be permeable to varied selected substances; for example, the membrane may be permeable to nutrients, proteins and other molecules produced by the cells, waste products, or the like. In other cases, the membrane may be gas impermeable. Some membranes are transparent to particular light (e.g. infrared, UV, or visible light; light of a wavelength with which a device utilizing the membrane interacts; visible light if not otherwise indicted). Where a membrane is substantially transparent, it absorbs no more than 50% of light, or in other embodiments no more than 25% or 10% of light, as described more fully herein. In some cases, a membrane may be both semipermeable and substantially transparent. The membrane, in one embodiment, may be used to divide a reaction site constructed and arranged to support cell culture from a second portion, for example, a reservoir. For example, a reaction site may be divided into three portions, four portions, or five portions. For instance, a reaction site may be divided into a first cell culture portion and a second cell culture portion flanking a first reservoir portion and two additional reservoir portions, one of which is separated by a membrane from the first cell culture portion and the other of which is separated by a membrane from the second cell culture portion. Of course, those of ordinary skill in the art will be able to design other arrangements, having varying numbers of cell culture portions, reservoir portions, and the like, as further described below.

As used herein, a “substantially transparent” material (for example, a membrane) is a material that allows electromagnetic radiation to be transmitted through the material without significant scattering, such that the intensity of electromagnetic radiation transmitted through the material is sufficient to allow the radiation to interact with a substance on the other side of the material, such as a chemical, biochemical, or biological reaction, or a cell. In some cases, the material is substantially transparent to incident electromagnetic radiation ranging between the infrared and ultraviolet ranges (including visible light) and, in particular, between wavelengths of about 400-410 nm and about 1,000 nm. In some cases, the material may be transparent to electromagnetic radiation between wavelengths of about 400-410 nm and about 800 nm, and in some embodiments, the material may be substantially transparent to radiation between wavelengths of about 450 nm and 700 nm. The substantially transparent material may be able to transmit electromagnetic radiation in some cases such that a majority of the radiation incident on the material passes through the material unaltered, and in some embodiments, at least about 50%, in other embodiments at least about 75%, in other embodiments at least about 80%, in still other embodiments at least about 90%, in still other embodiments at least about 95%, in still other embodiments at least about 97%, and in still other embodiments at least about 99% of the incident radiation is able to pass through the material unaltered. In certain cases, the material is at least partially transparent to electromagnetic radiation within the above-mentioned wavelength range to the extent necessary to promote and/or monitor a physical, chemical, biochemical, and/or biological reaction occurring within a reaction site, for example as previously described. In other embodiments, the material may be transparent to electromagnetic radiation within the above-mentioned wavelength range to the extent necessary to monitor, observe, stimulate and/or control a cell within the reaction site.

As used herein, a “reactor” is the combination of components including a reaction site, any chambers (including reaction chambers and ancillary chambers), channels, ports, inlets and/or outlets (i.e., leading to or from a reaction site), sensors, actuators, processors, controllers, membranes, and the like, which, together, operate to promote and/or monitor a biological, chemical, or biochemical reaction, interaction, operation, or experiment at a reaction site, and which can be part of a chip. For example, a chip may include at least 5, at least 10, at least 20, at least 50, at least 100, at least 500, or at least 1,000 or more reactors. Examples of reactors include chemical or biological reactors and cell culturing devices, as well as the reactors described in International Patent Application Serial No. PCT/US01/07679, published on Sep. 20, 2001 as WO 01/68257, incorporated herein by reference. Reactors can include one or more reaction sites or chambers. The reactor may be used for any chemical, biochemical, and/or biological purpose, for example, cell growth, pharmaceutical production, chemical synthesis, hazardous chemical production, drug screening, materials screening, drug development, chemical remediation of warfare reagents, or the like. For example, the reactor may be used to facilitate very small scale culture of cells or tissues. In one set of embodiments, a reactor of the invention comprises a matrix or substrate of a few millimeters to centimeters in size, containing channels with dimensions on the order of, e.g., tens or hundreds of micrometers. Reagents of interest may be allowed to flow through these channels, for example to a reaction site, or between different reaction sites, and the reagents may be mixed or reacted in some fashion. The products of such reactions can be recovered, separated, and treated within the system in certain cases.

As used herein, a “reaction site” is defined as a site within a reactor that is constructed and arranged to produce a physical, chemical, biochemical, and/or biological reaction during use of the reactor. More than one reaction site may be present within a reactor or a chip in some cases, for example, At least one reaction site, at least two reaction sites, at least three reaction sites, at least four reaction sites, at least 5 reaction sites, at least 7 reaction sites, at least 10 reaction sites, at least 15 reaction sites, at least 20 reaction sites, at least 30 reaction sites, at least 40 reaction sites, at least 50 reaction sites, at least 100 reaction sites, at least 500 reaction sites, or at least 1,000 reaction sites or more may be present within a reactor or a chip. The reaction site may be defined as a region where a reaction is allowed to occur; for example, the reactor may be constructed and arranged to cause a reaction within a channel, one or more chambers, at the intersection of two or more channels, etc. The reaction may be, for example, a mixing or a separation process, a reaction between two or more chemicals, a light-activated or a light-inhibited reaction, a biological process, and the like. In some embodiments, the reaction may involve an interaction with light that does not lead to a chemical change, for example, a photon of light may be absorbed by a substance associated with the reaction site and converted into heat energy or re-emitted as fluorescence. In certain embodiments, the reaction site may also include one or more cells and/or tissues. Thus, in some cases, the reaction site may be defined as a region surrounding a location where cells are to be placed within the reactor, for example, a cytophilic region within the reactor.

In some cases, the reaction site containing cells may include a region containing a gas (e.g., a “gas head space”), for example, if the reaction site is not completely filled with a liquid. The gas head space, in some cases, may be partially separated from the reaction site, through use of a gas-permeable or semi-permeable membrane. In some cases, the gas head space may include various sensors for monitoring temperature, and/or other reaction conditions.

Many embodiments and arrangements of the invention are described with reference to a chip, or to a reactor, and those of ordinary skill in the art will recognize that the invention can apply to either or both. For example, a channel arrangement may be described in the context of one, but it will be recognized that the arrangement can apply in the context of the other (or, typically, both: a reactor which is part of a chip). It is to be understood that all descriptions herein that are given in the context of a reactor or chip apply to the other, unless inconsistent with the description of the arrangement in the context of the definitions of “chip” and “reactor” herein.

In some embodiments, the reaction site may be defined by geometrical considerations. For example, the reaction site may be defined as a chamber in a reactor, a channel, an intersection of two or more channels, or other location defined in some fashion (e.g., formed or etched within a substrate that can define a reactor and/or chip). Other methods of defining a reaction site are also possible. In some embodiments, the reaction site may be artificially created, for example, by the intersection or union of two or more fluids (e.g., within one or several channels), or by constraining a fluid on a surface, for example, using bumps or ridges on the surface to constrain fluid flow. In other embodiments, the reaction site may be defined through electrical, magnetic, and/or optical systems. For example, a reaction site may be defined as the intersection between a beam of light and a fluid channel.

The volume of the reaction site can be very small in certain embodiments. Specifically, the reaction site may have a volume of less than one liter, less than about 100 ml, les than about 10 ml, less than about 5 ml, less than about 3 ml, less than about 2 ml, less than about 1 ml, less than about 500 microliters, less than about 300 microliters, less than about 200 microliters, less than about 100 microliters, less than about 50 microliters, less than about 30 microliters, less than about 20 microliters or less than about 10 microliters in various embodiments. The reaction site may also have a volume of less than about 5 microliters, or less than about 1 microliter in certain cases. The reaction site may have any convenient size and/or shape. In another set of embodiments, the reaction site may have a dimension that is 500 microns deep or less, 200 microns deep or less, or 100 microns deep or less.

In some cases, cells can be present at the reaction site. Sensor(s) associated with the chip or reactor, in certain cases, may be able to determine the number of cells, the density of cells, the status or health of the cell, the cell type, the physiology of the cells, etc. In certain cases, the reactor can also maintain or control one or more environmental factors associated with the reaction site, for example, in such a way as to support a chemical reaction or a living cell. In one set of embodiments, a sensor may be connected to an actuator and/or a microprocessor able to produce an appropriate change in an environmental factor within the reaction site. The actuator may be connected to an external pump, the actuator may cause the release of a substance from a reservoir, or the actuator may produce sonic or electromagnetic energy to heat the reaction site, or selectively kill a type of cell susceptible to that energy. The reactor can include one or more than one reaction site, and one or more than one sensor, actuator, processor, and/or control system associated with the reaction site(s). It is to be understood that any reaction site or a sensor technique disclosed herein can be provided in combination with any combination of other reaction sites and sensors.

As used herein, a “channel” is a conduit associated with a reactor and/or a chip (within, leading to, or leading from a reaction site) that is able to transport one or more fluids specifically from one location to another, for example, from an inlet of the reactor or chip to a reaction site, e.g., as further described below. Materials (e.g., fluids, cells, particles, etc.) may flow through the channels, continuously, randomly, intermittently, etc. The channel may be a closed channel, or a channel that is open, for example, open to the external environment surrounding the reactor or chip containing the reactor. The channel can include characteristics that facilitate control over fluid transport, e.g., structural characteristics (e.g., an elongated indentation), physical/chemical characteristics (e.g., hydrophobicity vs. hydrophilicity) and/or other characteristics that can exert a force (e.g., a containing force) on a fluid when within the channel. The fluid within the channel may partially or completely fill the channel. In some cases the fluid may be held or confined within the channel or a portion of the channel in some fashion, for example, using surface tension (i.e., such that the fluid is held within the channel within a meniscus, such as a concave or convex meniscus). The channel may have any suitable cross-sectional shape that allows for fluid transport, for example, a square channel, a circular channel, a rounded channel, a rectangular channel (e.g., having any aspect ratio), a triangular channel, an irregular channel, etc. The channel may be of any size within the reactor or chip. For example, the channel may have a largest dimension perpendicular to a direction of fluid flow within the channel of less than about 1000 micrometers in some cases, less than about 500 micrometers in other cases, less than about 400 micrometers in other cases, less than about 300 micrometers in other cases, less than about 200 micrometers in still other cases, less than about 100 micrometers in still other cases, or less than about 50 or 25 micrometers in still other cases. In some embodiments, the dimensions of the channel may be chosen such that fluid is able to freely flow through the channel, for example, if the fluid contains cells. The dimensions of the channel may also be chosen in certain cases, for example, to allow a certain volumetric or linear flowrate of fluid within the channel. In one embodiment, the depth of other largest dimension perpendicular to a direction of fluid flow may be similar to that of a reaction site to which the channel is in fluid communication with. Of course, the number of channels, the shape or geometry of the channels, and the placement of channels within the chip can be determined by those of ordinary skill in the art.

Chips of the invention may also include a plurality of inlets and/or outlets that can receive and/or output any of a variety of reactants, products, and/or fluids, for example, directed towards one or more reactors and/or reaction sites. In some cases, the inlets and/or outlets may allow the aseptic transfer of compounds. At least a portion of the plurality of inlets and/or outlets may be in fluid communication with one or more reaction sites within the chip. In some cases, the inlets and/or outlets may also contain one or more sensors and/or actuators, as further described below. Essentially, the chip may have any number of inlets and/or outlets from one to tens of hundreds that can be in fluid communication with one or more reactors and/or reaction sites. Less than 5 or 10 inlets and/or outlets may be provided to the reactor and/or reaction site(s) for certain reactions, such as biological or biochemical reactions. In some cases each reactor may have around 25 inlets and/or outlets, in other cases around 50 inlets and/or outlets, in still other cases around 75 inlets and/or outlets, or around 100 or more inlets and/or outlets in still other cases.

As one example, the inlets and/or outlets of the chip, directed to one or more reactors and/or reaction sites may include inlets and/or outlets for a fluid such as a gas or a liquid, for example, for a waste stream, a reactant stream, a product stream, an inert stream, etc. In some cases, the chip may be constructed and arranged such that fluids entering or leaving reactors and/or reaction sites do not substantially disturb reactions that may be occurring therein. For example, fluids may enter and/or leave a reaction site without affecting the rate of reaction in a chemical, biochemical, and/or biological reaction occurring within the reaction site, or without disturbing and/or disrupting cells that may be present within the reaction site. Examples of inlet and/or outlet gases may include, but are not limited to, CO₂, CO, oxygen, hydrogen, NO, NO₂, water vapor, nitrogen, ammonia, acetic acid, etc. As another example, an inlet and/or outlet fluid may include liquids and/or other substances contained therein, for example, water, saline, cells, cell culture medium, blood or other bodily fluids, antibodies, pH buffers, solvents, hormones, carbohydrates, nutrients, growth factors, therapeutic agents (or suspected therapeutic agents), antifoaming agents (e.g., to prevent production of foam and bubbles), proteins, antibodies, and the like. The inlet and/or outlet fluid may also include a metabolite in some cases. A “metabolite,” as used herein, is any molecule that can be metabolized by a cell. For example, a metabolite may be or include an energy source such as a carbohydrate or a sugar, for example, glucose, fructose, galactose, starch, corn syrup, and the like. Other example metabolites include hormones, enzymes, proteins, signaling peptides, amino acids, etc.

The inlets and/or outlets may be formed within the chip by any suitable technique known to those of ordinary skill in the art, for example, by holes or apertures that are punched, drilled, molded, milled, etc. within the chip or within a portion of the chip, such as a substrate layer. In some cases, the inlets and/or outlets may be lined, for example, with an elastomeric material. In certain embodiments, the inlets and/or outlets may be constructed using self-sealing materials that may be re-usable in some cases. For example, an inlet and/or outlet may be constructed out of a material that allows the inlet and/or outlet to be liquid-tight (i.e., the inlet and/or outlet will not allow a liquid to pass therethrough without the application of an external driving force, but may admit the insertion of a needle or other mechanical device able to penetrate the material under certain conditions). In some cases, upon removal of the needle or other mechanical device, the material may be able to regain its liquid-tight properties (i.e., a “self-sealing” material). Non-limiting examples of self-sealing materials suitable for use with the invention include, for example, polymers such as polydimethylsiloxane (“PDMS”), natural rubber, HDPE, or silicone materials such as Formulations RTV 108, RTV 615, or RTV 118 (General Electric, New York, N.Y.).

In some embodiments, the chip of the present invention may include very small elements, for example, sub-millimeter or microfluidic elements. For example, in some embodiments, the chip may include at least one reaction site having a cross sectional dimension of no greater than, for example, 100 mm, 80 mm, 50 mm, or 10 mm. In some embodiments, the reaction site may have a maximum cross section no greater than, for example, 100 mm, 80 mm, 50 mm, or 10 mm. As used herein, the “cross section” refers to a distance measured between two opposed boundaries of the reaction site, and the “maximum cross section” refers to the largest distance between two opposed boundaries that may be measured. In other embodiments, a cross section or a maximum cross section of a reaction site may be less than 5 mm, less than 2 mm, less than 1 mm, less than 500 micrometers, less than 300 micrometers, less than 100 micrometers, less than 10 micrometers, or less than 1 micrometer or smaller. As used herein, a “microfluidic chip” is a chip comprising at least one fluidic element having a sub-millimeter cross section, i.e., having a cross section that is less than 1 mm. As one particular non-limiting example, a reaction site may have a generally rectangular shape, with a length of 80 mm, a width of 10 mm, and a depth of 5 mm.

While one reaction site may be able to hold and/or react a small volume of fluid as described herein, the technology associated with the invention also allows for scalability and parallelization. With regard to throughput, an array of many reactors and/or reaction sites within a chip, or within a plurality of chips, can be built in parallel to generate larger capacities. For example, a plurality of chips (e.g. at least about 10 chips, at least about 30 chips, at least about 50 chips, at least about 75 chips, at least about 100 chips, at least about 200 chips, at least about 300 chips, at least about 500 chips, at least about 750 chips, or at least about 1,000 chips or more) may be operated in parallel, for example, through the use of robotics, for example which can monitor or control the chips automatically. Additionally, an advantage may be obtained by maintaining production capacity at the small scale of reactions typically performed in the laboratory, with scale-up via parallelization. It is a feature of the invention that many reaction sites may be arranged in parallel within a reactor of a chip and/or within a plurality of chips. Specifically, at least five reaction sites can be constructed to operate in parallel, or in other cases at least about 7, about 10, about 30, about 50, about 100, about 200, about 500, about 1,000, about 5,000, about 10,000, about 50,000, or even about 100,000 or more reaction sites can be constructed to operate in parallel, for example, in a high-throughput system. In some cases, the number of reaction sites may be selected so as to produce a certain quantity of a species or product, or so as to be able to process a certain amount of reactant. In certain cases the parallelization of the chips and/or reactors may allow many compounds to be screened simultaneously, or many different growth conditions and/or cell lines to be tested and/or screened simultaneously. Of course, the exact locations and arrangement of the reaction site(s) within the reactor or chip will be a function of the specific application.

Additionally, any embodiment described herein can be used in conjunction with a collection chamber connectable ultimately to an outlet of one or more reactors and/or reaction sites of a chip. The collection chamber may have a volume of greater than 10 milliliters or 100 milliliters in some cases. The collection chamber, in other cases, may have a volume of greater than 100 liters or 500 liters, or greater than 1 liter, 2 liters, 5 liters, or 10 liters. Large volumes may be appropriate where the reactors and/or reaction sites are arranged in parallel within one or more chips, e.g., a plurality of reactors and/or reaction sites may be able to deliver a product to a collection chamber.

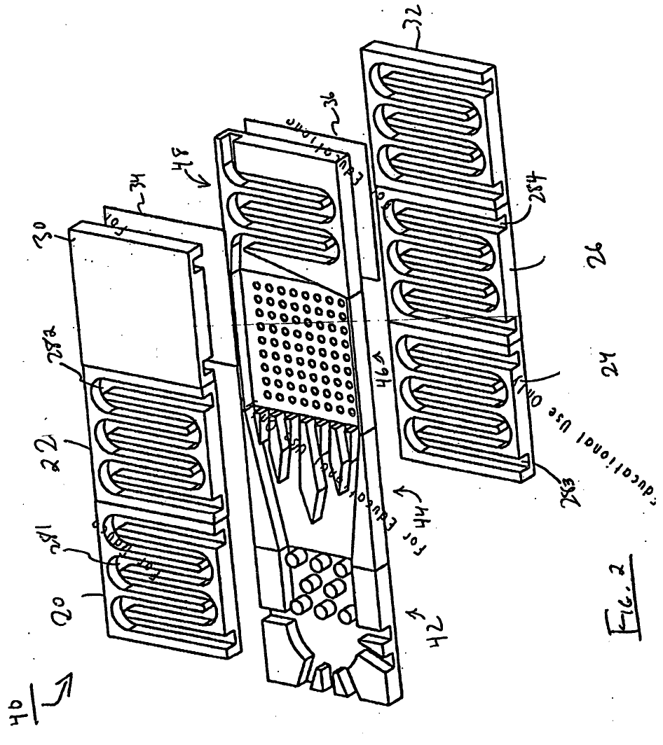

In some embodiments, the reaction site(s) and/or the channels in fluidic communication with the reaction site(s) are free of active mixing elements. In these embodiments, the reactor of the chip can be constructed in such a way as to cause turbulence in the fluids provided through the inlets and/or outlets, thereby mixing and/or delivering a mixture of the fluids, preferably without active mixing, where mixing is desired. Specifically, the reactor and/or reaction site(s) may include a plurality of obstructions in the flow path of the fluid that causes fluid flowing through the flow path to mix, for example, as shown in mixing unit 42 in FIG. 2. These obstructions can be of essentially any geometrical arrangement for example, a series of pillars. As used herein, “active mixing elements” is meant to define mixing elements such as blades, stirrers, or the like, which are movable relative to the reaction site(s) and/or channels themselves, that is, movable relative to portion(s) of the reactor defining a reaction site a or a channel.

Chips of the invention can be constructed and arranged such that they are able to be stacked in a predetermined, pre-aligned relationship with other, similar chips, such that the chips are all oriented in a predetermined way (e.g., all oriented in the same way) when stacked together. When a chip of the invention is designed to be stacked with other, similar chips, the chip often can be constructed and arranged such that at least a portion of the chip, such as a reaction site, is in fluidic communication with one or more of the other chips and/or reaction sites within other chips. This arrangement may find use in parallelization of chips, as discussed herein.

In one set of embodiments, the chip is constructed and arranged such that the chip is able to be stably connected to a microplate, for example, as defined in the 2002 SPS/ANSI proposed standard (e.g., a microplate having dimensions of roughly 127.76±0.50 mm by 85.48±0.50 mm). As used herein, “stably connected” refers to systems in which two components are connected such that a specific motion or force is necessary to disconnect the two components from each other, i.e., the two components cannot be dislodged by random vibration or displacement (e.g., accidentally lightly bumping a component). The components can be stably connected by way of pegs, screws, snap-fit components, matching sets of indentations and protrusions, or the like. A “microplate” is also sometimes referred to as a “microtiter” plate, a “microwell” plate, or other similar terms known to the art. The microplate may include any number of wells. For example, as is typically used commercially, the microplate may be a six-well microplate, a 24-well microplate, a 96-well microplate, a 384-well microplate, or a 1,536-well microplate. The wells may be of any suitable shape, for example, cylindrical or rectangular. The microplate may also have other numbers of wells and/or other well geometries or configurations, for instance, in certain specialized applications.

FIGS. 3A-3C illustrate one set of embodiments of the invention in which one or more reaction sites may be positioned in association with a chip such that, when the chip is stably connected to other chips and/or microplates, one or more reaction sites of the chip are positioned or aligned to be in chemical, biological, or biochemical communication with, or chemically, biologically, or biochemically connectable with one or more reaction sites of the other chip(s) and/or one or more wells of the microplate(s). “Alignment,” in this context, can mean complete alignment, such that the entire area of the side of a reaction site adjacent another reaction site or well completely overlaps the other reaction site or well, and vice versa, or at least a portion of the reaction site can overlap at least a portion of an adjacent reaction site or well. “Chemically, biologically, or biochemically connectable” means that the reaction site is in fluid communication with another reaction site or well (i.e., fluid is free to flow from one to the other); or is fluidly connectable to the other site or well (e.g., the two are separated from each other by a wall or other component that can be punctured or ruptured, or a valve in a conduit connecting the two can be opened); or the reaction site and other site or well are arranged such that at least some chemical, biological, or biochemical species can migrate from one to the other, e.g., across a semipermeable membrane. As examples, a chip may have six reaction sites that are constructed and arranged to be aligned with the six wells of a 6-well microplate when the chip is stably connected with the microplate (e.g., positioned on top of the microplate), a chip having 96 reaction sites may be constructed and arranged such that the 96 wells are constructed and arranged to be aligned with the 96 wells of a 96-well microplate when the chip is stably connected with the microplate, etc. Of course, in some cases, the chip may be constructed and arranged such that a single reaction site of the chip is aligned with more than one microplate well and/or more than one other reaction site, and/or such that more than one microplate well and/or more than one other reaction site is aligned with a single reaction site of the chip.

Chips of the invention also may be constructed and arranged such that at least one reaction site and/or reactor of the chip is in fluid communication with, and/or chemically, biologically, or biochemically connectable to an apparatus constructed and arranged to address at least one well of a microplate, for example, an apparatus that can add species to and/or remove species from wells of microplates, and/or can test species within wells of a microplate. In this arrangement, the apparatus may add and/or remove species to/from a reaction site of a chip, and/or test species at reaction sites. In this embodiment, the reaction sites typically are arranged in alignment with wells of the microplate.

With reference to FIGS. 3A and 3B, examples are shown in which inventive chip 120 may be stably connected to commercially-available microplate 123. In FIG. 3A, chip 120 may be positioned such that at least some of reaction sites 125 of chip 120 are aligned with, and/or connectable with at least some of wells 127 of microplate 123 when chip 120 is stably connected to microplate 123. Similarly, in FIG. 3B, chip 120 may be constructed and arranged such that, when stably connected to microplate 23, at least some of reaction sites 125 are aligned with, and/or connectable with at least a portion of wells 127 on microplate 123. In FIG. 3C, another embodiment of the invention is shown where chips 130, 131, . . . 132, are constructed and arranged such that the chips can be stably connected to each other. In some cases, chips 130, 131, . . . 132 are constructed and arranged such that, when stably connected to each other, reaction site 135 of chip 130 is aligned with one or more other reaction sites on other chips, for example, with reaction site 136 in chip 131, and/or reaction site 137 in chip 132.

Chips of the invention can be substantially liquid-tight in one set of embodiments. As used herein, a “substantially liquid-tight chip” or a “substantially liquid-tight reactor” is a chip or reactor, respectively, that is constructed and arranged, such that, when the chip or reactor is filled with a liquid such as water, the liquid is able to enter or leave the chip or reactor solely through defined inlets and/or outlets of the chip or reactor, regardless of the orientation of the chip or reactor, when the chip is assembled for use. In this set of embodiments, the chip is constructed and arranged such that when the chip or reactor is filled with water and the inlets and/or outlets sealed, the chip or reactor has an evaporation rate of less than about 100 microliters per day, less than about 50 microliters per day, or less than about 20 microliters per day. In certain cases, a chip or reactor will exhibit an unmeasurable, non-zero amount of evaporation of water per day. The substantially liquid-tight chip or reactor can have a zero evaporation rate of water in other cases.

Chips of the invention can be fabricated using any suitable manufacturing technique for producing a chip having one or more reactors, each having one or multiple reaction sites, and the chip can be constructed out of any material or combination of materials able to support a fluidic network necessary to supply and define at least one reaction site. Non-limiting examples of microfabrication processes include wet etching, chemical vapor deposition, deep reactive ion etching, anodic bonding, injection molding, hot pressing, and LIGA. For example, the chip may be fabricated by etching or molding silicon or other substrates, for example, via standard lithographic techniques. The chip may also be fabricated using microassembly or micromachining methods, for example, stereolithography, laser chemical three-dimensional writing methods, modular assembly methods, replica molding techniques, injection molding techniques, milling techniques, and the like as are known by those of ordinary skill in the art. The chip may also be fabricated by patterning multiple layers on a substrate (which may be the same or different), for example, as further described below, or by using various known rapid prototyping or masking techniques. Examples of materials that can be used to form chips include polymers, silicones, glasses, metals, ceramics, inorganic materials, and/or a combination of these. The materials may be opaque, semi-opaque translucent, or transparent, and may be gas permeable, semi-permeable or gas impermeable. In some cases, the chip may be formed out of a material that can be etched to produce a reactor, reaction site and/or channel. For example, the chip may comprise an inorganic material such as a semiconductor, fused silica, quartz, or a metal. The semiconductor material may be, for example, but not limited to, silicon, silicon nitride, gallium arsenide, indium arsenide, gallium phosphide, indium phosphide, gallium nitride, indium nitride, other Group III/V compounds, Group II/VI compounds, Group III/V compounds, Group IV compounds, and the like, for example, compounds having three or more elements. The semiconductor material may also be formed out of combination of these and/or other semiconductor materials known in the art. In some cases, the semiconductor material may be etched, for example, via known processes such as lithography. In certain embodiments, the semiconductor material may have the from of a wafer, for example, as is commonly produced by the semiconductor industry.

In some embodiments, a chip of the invention may be formed from or include a polymer, such as, but not limited to, polyacrylate, polymethacrylate, polycarbonate, polystyrene, polyethylene, polypropylene, polyvinylchloride, polytetrafluoroethylene, a fluorinated polymer, a silicone such as polydimethylsiloxane, polyvinylidene chloride, bis-benzocyclobutene (“BCB”), a polyimide, a fluorinated derivative of a polyimide, or the like. Combinations, copolymers, or blends involving polymers including those described above are also envisioned. The chip may also be formed from composite materials, for example, a composite of a polymer and a semiconductor material.

In some embodiments, the chip, or at least a portion thereof, is rigid, such that the chip is sufficiently sturdy in order to be handled by commercially-available microplate-handling equipment, and/or such that the chip does not become deformed after routine use. Those of ordinary skill in the art are able to select materials or a combination of materials for chip construction that meet this specification, while meeting other specifications for use for which a particular chip is intended. In other embodiments, however, the chip may be semi-rigid or flexible.

In certain embodiments, the chip may include a sterilizable material. For example, the chip may be sterilizable in some fashion to kill or otherwise deactivate biological cells (e.g., bacteria), viruses, etc. therein, before the chip is used or re-used. For instance, the chip may be sterilized with chemicals, radiated (for example, with ultraviolet light and/or ionizing radiation), heat-treated, or the like. Appropriate sterilization techniques and protocols are known to those of ordinary skill in the art. For example, in one embodiment, the chip is autoclavable, i.e., the chip is constructed and arranged out of materials able to withstand commonly-used autoclaving conditions (e.g., exposure to temperatures greater than about 100° C. or about 120° C., often at elevated pressures, such as pressures of at least one atmosphere), such that the chip, after sterilization, does not substantially deform or otherwise become unusable. Other examples of sterilization techniques include exposure to ozone, alcohol, pheloics, halogens, heavy metals (e.g., silver nitrate), detergents, quatanary ammonium components, ethylene oxide, CO₂, aldehydes, etc. In another embodiment, the chip is able to withstand ionizing radiation, for example, short wavelength, high-intensity radiation, such as gamma rays, electron-beams, or X-rays. In some cases, ionizing radiation may be produced from a nuclear reaction, e.g., from the decay of ⁶⁰Co or¹³⁷CS.

In one set of embodiments, at least a portion of the chip may be fabricated without the use of adhesive materials. For example, at least two components of a chip (e.g., two layers of the chip if the chip is a multi-layered structure, a layer or substrate of the chip and a membrane, two membranes, an article of the chip and a component of a microfluidic system, etc.) may be fastened together without the use of an adhesive material. For example, the components may be connected by using methods such as heat sealing, sonic welding, via application of a pressure-sensitive material, and the like. In one embodiment, the components may be held in place mechanically. For example, screws, posts, cantilevers, matching indentations, etc. may be used to mechanically hold the chip (or a portion thereof) together. In other embodiments, the two components of the chip may be joined together using techniques such as, but not limited to, heat-sealing methods (e.g., or more components of the chip may be heated to a temperature greater than the glass transition temperature or the melting temperature of the component before being joined to other components), or sonic welding techniques (e.g., vibration energy such as sound energy may be applied to one or more components of the chip, allowing the components to at least partially liquefy or soften).

In one embodiment, two components of the chip may be fastened via a heat-sealing method. For example, one or more components of the chip may be heated to a temperature greater than the glass transition temperature or the melting temperature of the component (i.e., temperatures at which the component softens or begins to liquefy). The components can be placed in contact with each other and allowed to cool to below the glass transition temperature or the melting temperature, thus allowing the components to become fastened together.

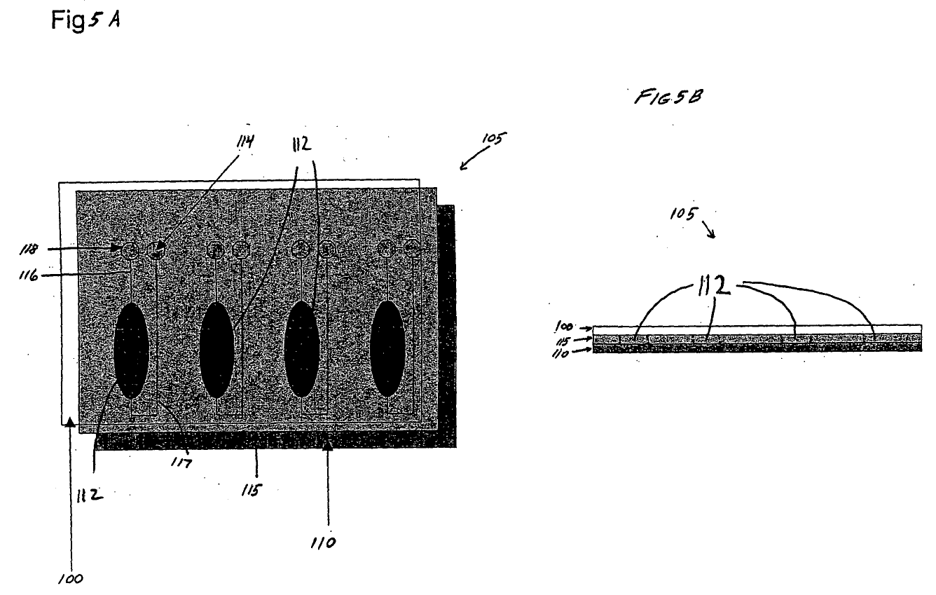

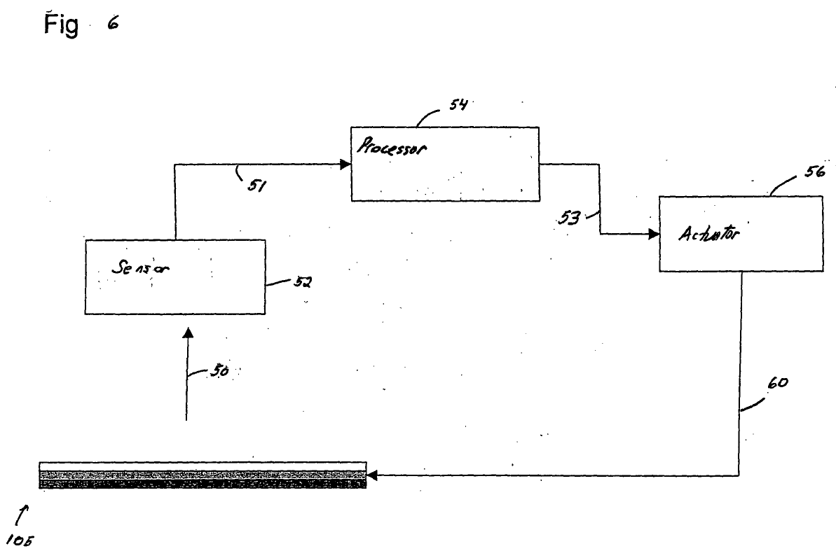

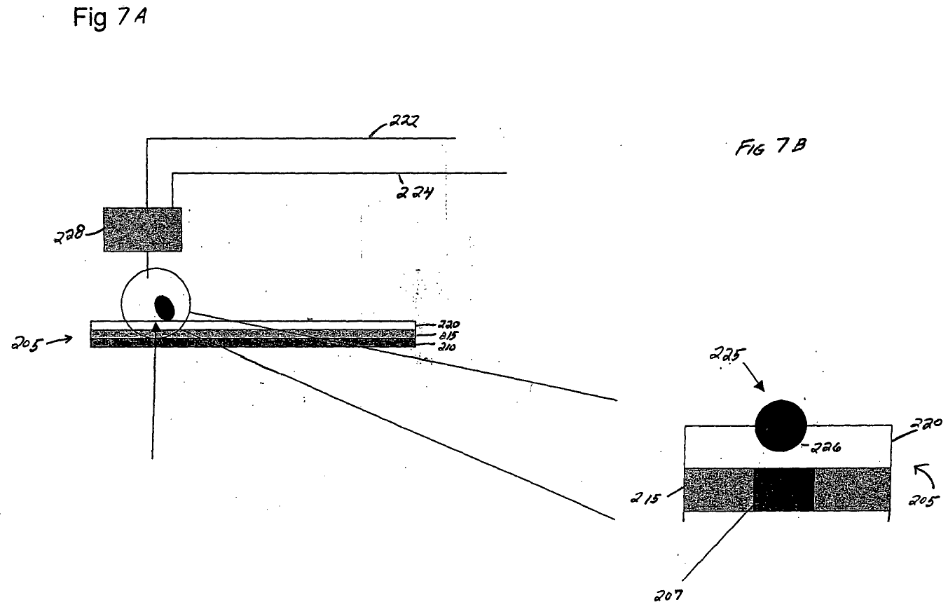

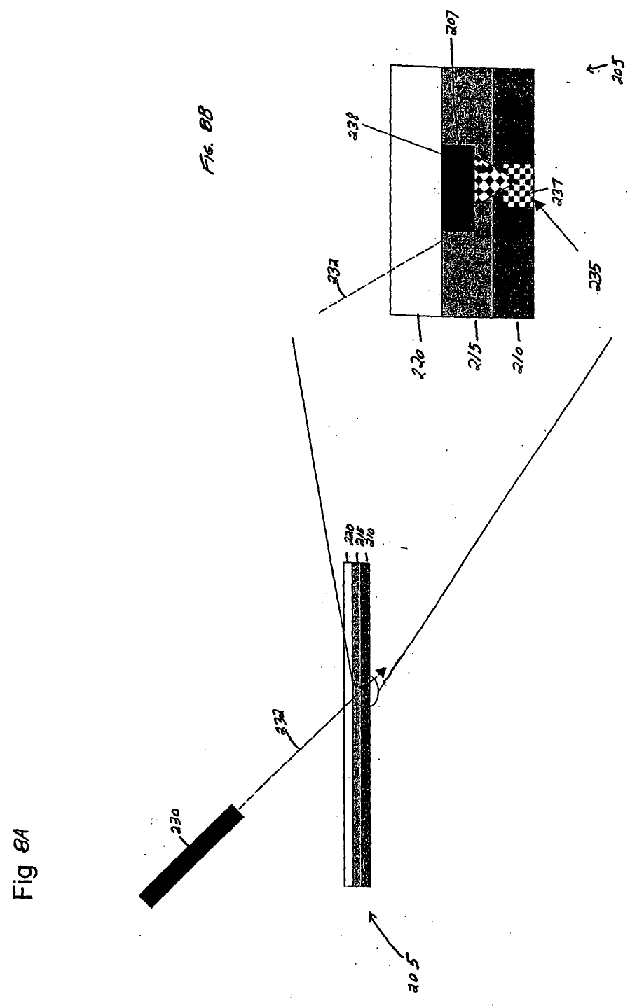

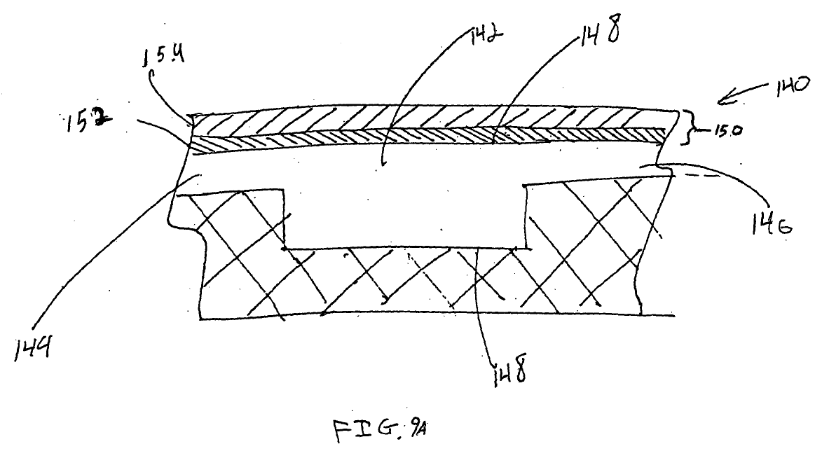

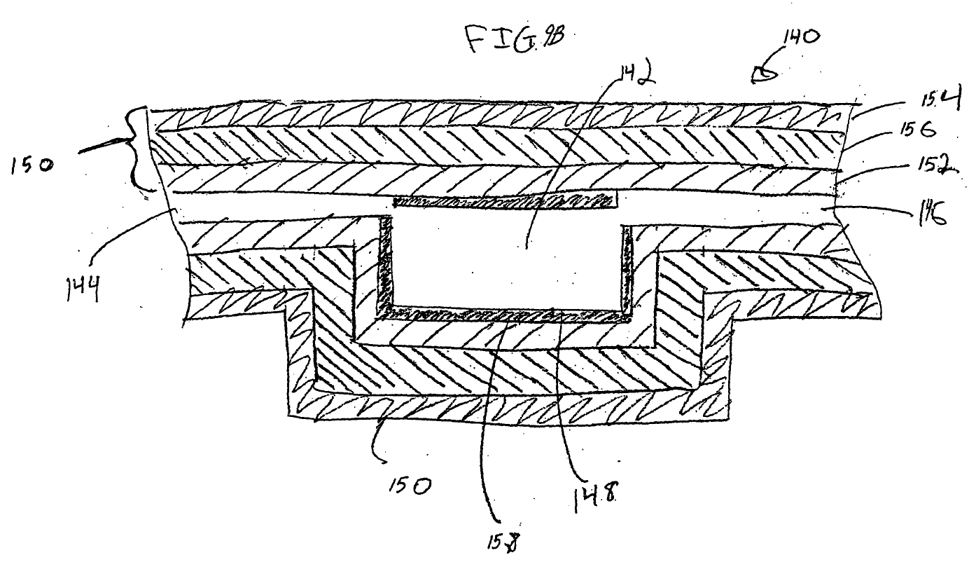

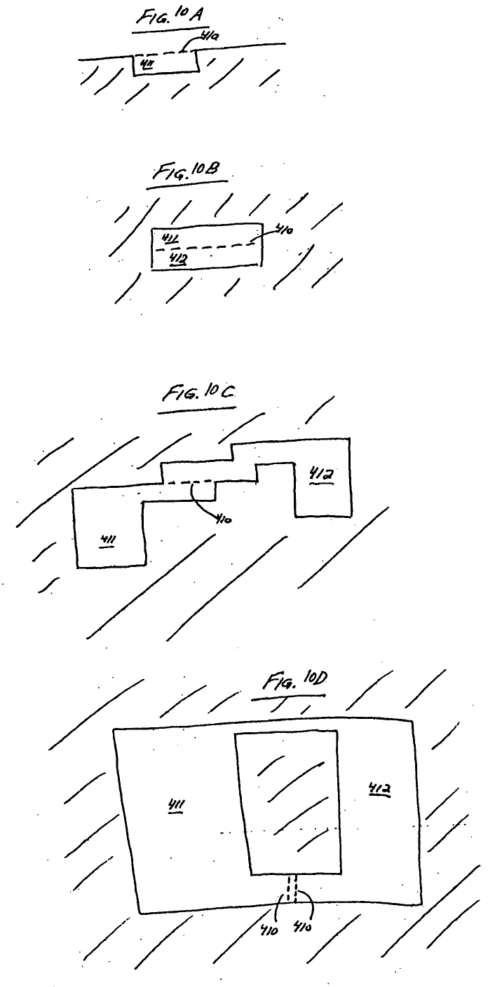

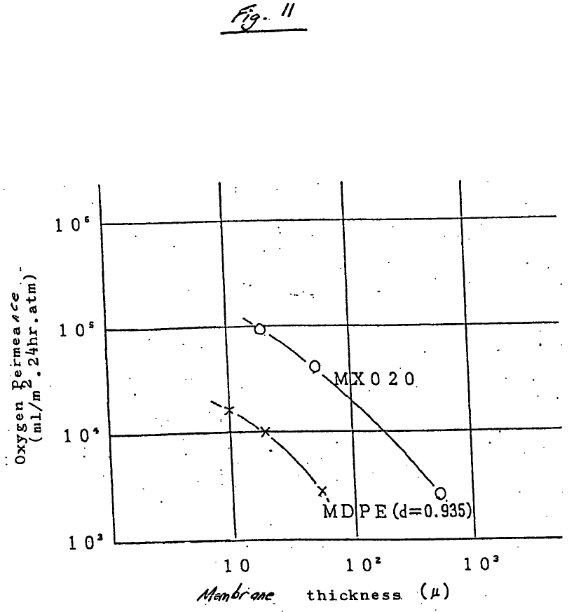

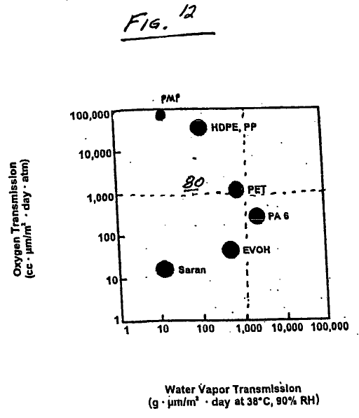

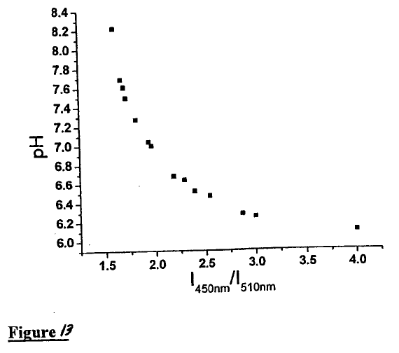

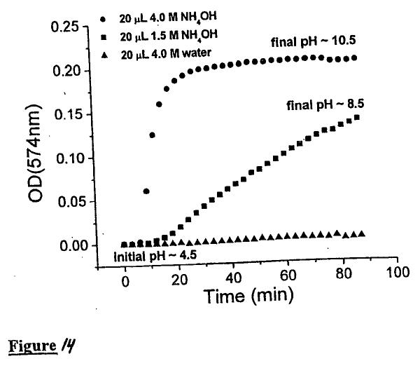

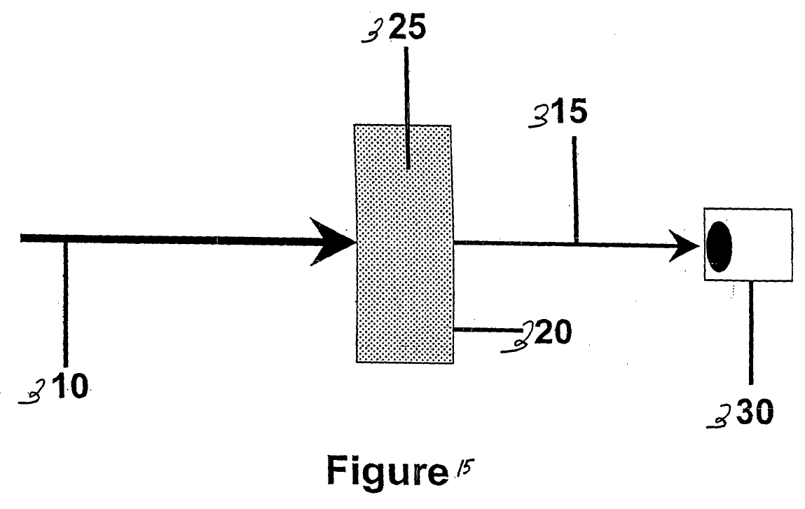

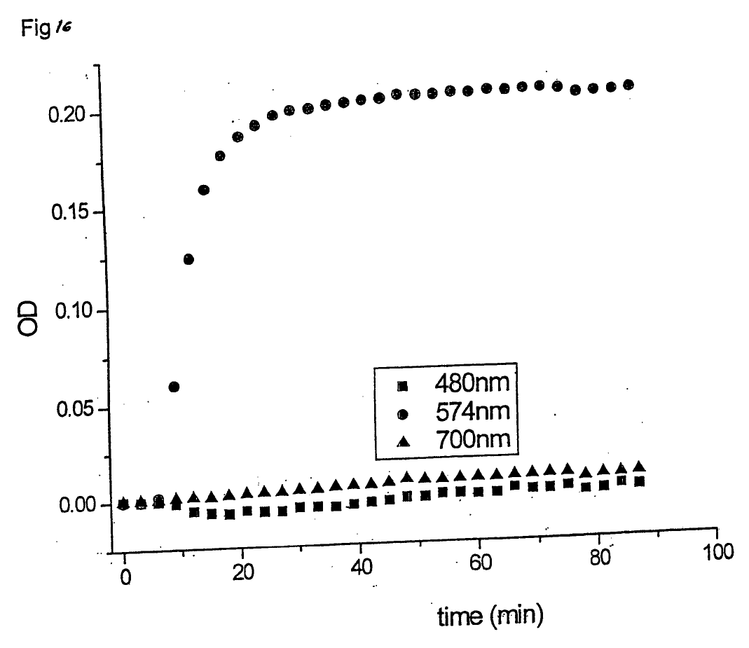

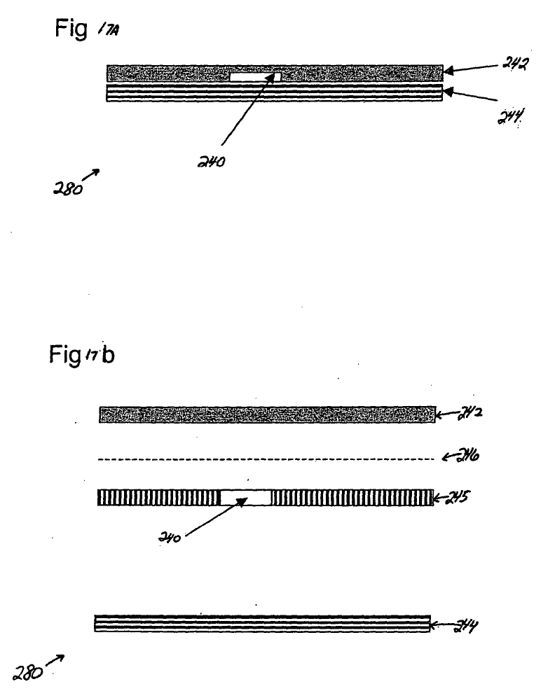

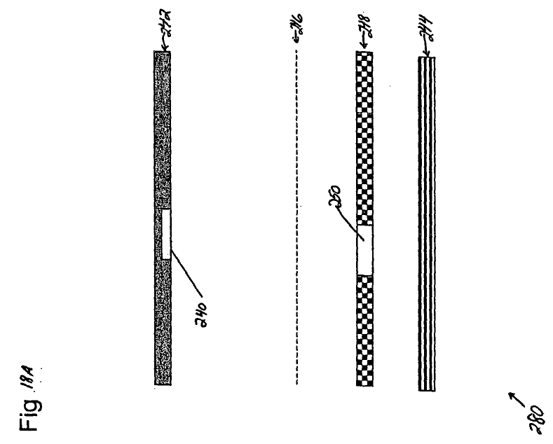

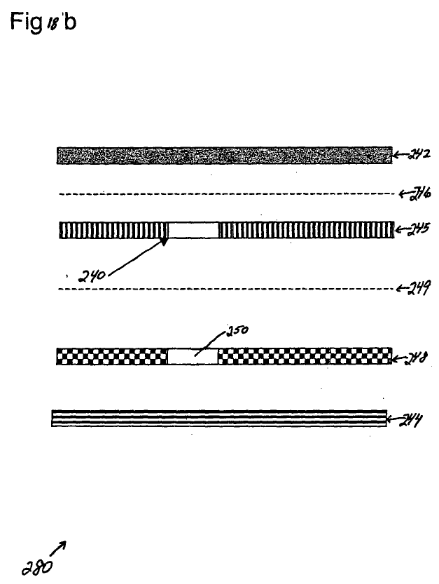

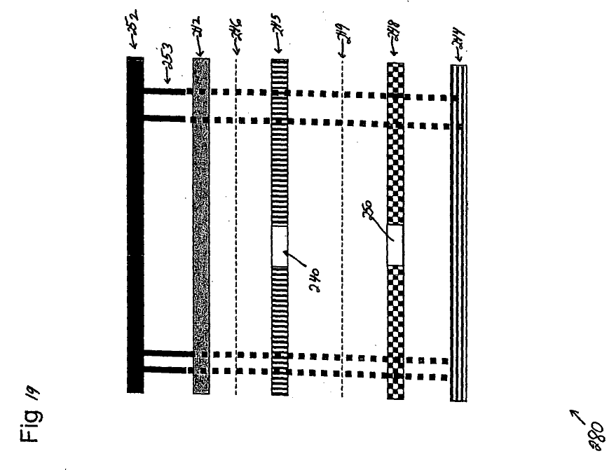

In another embodiment, the two components can be fastened via sonic welding techniques. As one example, vibration energy (e.g., sound energy) may be applied to one or more components of the chip. The applied vibration energy causes the component(s), or at least a portion of the component(s), to at least partially liquefy or soften. The components can then be placed together. The vibration energy may then be stopped, thus allowing the components to become fastened together. In some cases, the components may be designed such that the vibration energy is able to be concentrated into certain regions of the component (an “energy director” region), such that only the energy director region of the component is able to liquefy under the influence of the vibration energy. For example, as shown in FIG. 4A (side) and FIG. 4B (top), a side view of a component 75 of the chip is illustrated, showing energy director region 73. When vibration energy is applied to component 75, a substantial fraction of the energy can be concentrated in the energy director region 73, allowing at least a portion of energy director 73 to soften or liquefy. The softened and/or liquefied region may then be connected to other components of the chip and allowed to harden, thus allowing two components of the chip to be fastened together, for instance, as is shown in FIG. 4C, where component 75 has been fastened to component 77.

In another set of embodiments, two or more components of the chip may be joined using an adhesive material. As used herein, an “adhesive material” is given its ordinary meaning as used in the art, i.e., an auxiliary material able to fasten or join two other materials together. Non-limiting examples of adhesive materials suitable for use with the invention include silicone adhesives such as pressure-sensitive silicone adhesives, neoprene-based adhesives, and latex-based adhesives. The adhesive may be applied to one or more components of the chip using any suitable method, for example, by applying the adhesive to a component of the chip as a liquid or as a semi-solid material such as a viscoelastic solid. For example, in one embodiment, the adhesive may be applied to the component(s) using transfer tape (e.g., a tape having adhesive material attached thereto, such that, when the tape is applied to the component, the adhesive, or at least a portion of the adhesive, remains attached to the component when the tape is removed from the component). In one set of embodiments, the adhesive may be a pressure-sensitive adhesive, i.e., the material is not normally or substantially adhesive, but becomes adhesive and/or increases its adhesive strength under the influence of pressure, for example, a pressure greater than about 6 atm or about 13 atm (about 100 psi or about 200 psi). Non-limiting examples of pressure-sensitive adhesives include AR Clad 7876 (available from Adhesives Research, Inc., Glen Rock, Pa.) and Trans-Sil Silicone PSA NT-1001 (available from Dielectric Polymers, Holyoke, Mass.)

In another embodiment, the adhesive may be applied to at least a component of the chip using a solvent-bonding system. In a solvent-bonding system, one or more components of the chip are placed in an environment rich in solvent vapor, i.e., the environment that the component(s) is placed in is saturated or supersaturated with a solvent, such that the solvent is able to condense onto the component(s) placed within the environment under suitable conditions (e.g., when the pressure and/or the temperature is lowered). The components can then be contacted together within the environment and allowed to fasten together, for example, when the environment (including solvent) is removed. As one specific example, two polycarbonate components of a chip of the invention may be fastened together in a methylene chloride environment. For example, a thin layer of a solvent, i.e. methylene chloride or the like, may be applied to a surface. The two surfaces to be joined may then be pressed and/or clamped together under pressure to ensure bonding.

In some embodiments of the invention, the chip may be constructed and arranged such that one or more reaction sites can be defined, at least in part, by two or more components fastened together as previously described (i.e., with or without an adhesive). In some cases, a reaction site may be free of any adhesive material adjacent to or otherwise in contact with one or more surfaces defining the reaction site, and this can be advantageous, for instance, when an adhesive might otherwise leach into fluid at the reaction site. Of course, an adhesive may be used elsewhere in the chip, for example, in other reaction sites. Similarly, in certain cases, a reaction site may be constructed using adhesive materials, such that at least a portion of the adhesive material used to construct the reaction site remains within the chip such that it is adjacent to or otherwise remains in contact with one or more surfaces defining the reaction site. Of course, other components of the chip may be constructed without the use of adhesive materials, as previously discussed.

Referring now to FIG. 2, one example of a microfluidic chip 40 of the invention is shown. Chip 40 includes four general units, including a mixing unit 42, heating/dispersion unit 44, reaction site 46, and separation unit 48. One or more sensors, processors, and/or actuators (not shown) can optionally be included in sensing or actuating communication with the chip, respectively. “Sensing communication” and “actuating communication,” as used herein, means that a sensor or actuator, respectively, is positioned anywhere in association with the chip such that the environment of the reaction site and/or the content of the reaction site can be determined and/or controlled. A sensor or actuator can be included within the chip, for example embedded within or integrally connected to the reaction site, positioned within or on the chip, or positioned remotely from the chip but with physical, electrical, and/or optical connection with the reaction site so as to be able to sense or actuate a factor within the reaction site. For example, a sensor may be free of any physical connection with a chip, but may be positioned so as to detect the results of interaction of electromagnetic radiation, such as infrared, ultraviolet, or visible light, which has been directed toward a reaction site and has passed through the site or has been reflected or diffracted by the site. As another example, a sensor may be positioned on or within a chip, and may sense activity at a reaction site by being connected optically to the reaction site via a waveguide. The chip can be similarly directly or indirectly connected to a network or a control system for overall control of detection and actuation. Sensing and actuating communication can also be provided where the reaction site is in communication with a sensor or actuator fluidly, optically or visually, thermally, pneumatically, electronically, or the like, so as to be able to sense a condition of the reaction site and/or the content of the site. As one example, the sensor may be positioned downstream of one of the outlets, or behind a membrane or a transparent cover separating the reaction site from the sensor. Additional discussion of sensing and actuating arrangements is provided below.

FIG. 5 illustrates another embodiment of the invention. FIG. 5A illustrates a top view and FIG. 5B illustrates a side view of chip 105. In this embodiment, chip 105 is composed of three layers of material, namely, top layer 100 (which is transparent in the embodiment illustrated), middle layer 115, and lower layer 110. Of course, in other embodiments of the invention, chip 105 may have more or fewer layers of material (e.g., including only 1 layer), depending on the specific application. In the embodiment shown in FIG. 5, middle layer 115 has one or more void spaces 112, defining a plurality of predetermined reaction sites as discussed below. One or more channels 116, 117 may also be defined within middle layer 115, in fluid communication with reaction site 112.

In some cases, one or more ports 114, 118 may allow external access to the channels, for example through upper layer 100.

Upper layer 100 may cover or at least partially cover middle layer 115, thereby in part defining reaction site(s) 112. In some cases, upper layer 100 may be permeable to a gas or liquid, for example, in cases where a gas or liquid agent is allowed to permeate or penetrate through upper layer 100. For instance, upper layer 100 may be formed from a polymer such as PDMS or silicone, which may be thin enough to allow detectable or measurable gaseous transport therethrough. In some cases, gaseous transport through upper layer 100 may be possible, while the transport of a liquid through upper layer 100 is not generally possible within a reasonable time frame. In certain cases, upper layer 100 may also be substantially transparent or translucent, for example, in embodiments where light is used to initiate a reaction or activate a process (e.g., within the reaction site). In some cases, upper layer 100 may be formed from a polymer that allows a gaseous pH-altering agent to permeate across. In certain instances, upper layer 100 may be formed of a material that is self-sealing, i.e., the material may be penetrated by a solid object but generally regains its shape after such penetration. For example, upper layer 100 may be formed of an elastomeric material which may be penetrated by a mechanical device such as a needle, but which sealingly closes once the needle or other mechanical device is withdrawn.

Middle layer 115 includes four void spaces in the embodiment illustrated in FIG. 5. Of course, in other embodiments, more or fewer void spaces may be present within middle layer 115. In the embodiment illustrated in FIG. 5, void space in middle layer 115, along with upper layer 100 and lower layer 110, may define reaction site 112. In the embodiment of FIG. 5, there are four reaction sites 112, which are substantially identical; however, in other embodiments of the invention, more or fewer predetermined reaction sites may exist, and the reaction sites may each be the same or different. In the embodiment shown, each void space is substantially identical and has two fluid channels 116, 117 in communication with the void space. Of course, in other embodiments of the invention, there may be more or fewer channels running throughout the chip. In the embodiment of FIG. 5, fluid channel 116 is connected to port 118 in layer 115, and fluid channel 117 is connected to port 114 in layer 115; in other embodiments, of course, fluid channels 116 and 118 may fluidly connect one or more reaction sites to each other, to one or more fluid ports, and/or to one or more other components within chip 105. Ports 114 and/or 118 may be used to introduce or withdraw fluids or other substances from the reactor in some cases. In some embodiments of the invention, reaction site 112 and/or one or more fluidic channels may be defined, for example, in one or more layers of the chip, for example, solely within one layer, at a junction between two layers, in a void space that spans three layers, etc.

Ports 114 and 118 may be in fluid communication with one or more reaction site(s) 112. Ports 114 and 118 may be accessible, in some cases, by inserting a needle or other mechanical device through upper layer 100. For example, in some cases, upper layer 100 may be penetrated, or a space in upper layer 100 may permit external access to ports 114 and/or 118. In some cases, upper layer 100 may be composed of a flexible or elastomeric material, which may be self-sealing in some cases. In certain instances, upper layer 100 may have a passage formed therein that allows direct or indirect access to ports 114 and/or 118, or ports 114 and/or 118 may be formed in upper layer 100 and connected to channels 116 and 117 through channels defined within layer 100.

Lower layer 110 forms the bottom of chip 105, as illustrated in FIG. 5. As previously described, parts of lower layer 110 in part may define reaction site 112 in certain instances. In some cases, lower layer 110 may be formed of a relatively hard or rigid material, which may give relatively rigid structural support to chip 105. Of course, in other embodiments, lower layer 110 may be formed of a flexible or elastomeric material (i.e., non-rigid). In some cases, lower layer 110 may contain one or more channels defined therein and/or one or more ports defined therein. In some cases, material defining a boundary of the reaction site, such as lower layer 110 (or upper layer 100), may contain salts and/or other materials, for example, in cases where the materials are reacted in some fashion to produce an agent that is allowed to be transported to or proximate reaction site 112. The agent may be any agent as previously discussed, for instance, a gas, a liquid, an acid, a base, a tracer compound, a small molecule (e.g., a molecule with a molecular weight of less than about 1000 Da-1500 Da), a drug, a protein, or the like, and transport may occur by any suitable mechanism, for example, diffusion (natural or facilitated) or percolation. In one embodiment, the agent is produced by a thermal decomposition reaction that may be externally initiated, for example, using electric current or light (e.g., with a laser). In certain other cases, material defining a boundary of the reaction site, such as lower layer 110 or upper layer 100, may contain one or more reservoirs of agents that are not in fluidic contact with reaction site 112, but where the agents may be transported to or proximate the reaction site, for example, by creating at least one fluidic connection between a reservoir and a reaction site. The transport may be externally controlled or driven in some cases, e.g., using an electric or magnetic field to direct fluid movement. Of course, in still other cases, lower layer 110 and/or upper layer 100 may not contain any agents or other reservoirs.

It should be understood that the chips and reactors of the present invention may have a wide variety of different configurations. For example, the chip may be formed from a single material, or the chip may contain more than one type of reactor, reservoir and/or agent; In some cases, the chip may contain more than one system able to alter one or more environmental factor(s) within one or more reaction sites within the chip. For example, the chip may contain a sealed reservoir and an upper layer that a non-pH-neutral gas is able to permeate across.

Chips of the invention can be constructed and arranged so as to be able to detect or determine one or more environmental conditions associated with a reaction site of the reactor, for example, using a sensor. In some cases, each reaction site may be independently determined. Detection of the environmental condition may occur, for example, by means of a sensor which may be positioned within the reaction site, or positioned proximate the reaction site, i.e., positioned such that the sensor is in communication with the reaction site in some manner. In some cases, such detection may occur in real-time. The sensor may be, for example, a pH sensor, an optional sensor, an oxygen sensor, a sensor able to detect the concentration of a substance, or the like. Other examples of sensors are further described below. The sensor may be embedded and integrally connected with the chip (e.g., within a component defining at least a portion of the reaction site a channel in fluidic communication with the reaction site, etc.), or separate from the chip in some cases (e.g., within sensing communication). Also, the sensor may be integrally connected to or separate from the reaction site in certain embodiments.

As used herein, an “environmental factor” or an “environmental condition” is a detectable and/or measurable condition (e.g., by a sensor) of the environment within and/or associated with a reaction site, such as the temperature or pressure. The factor or condition may be detected and/or measured within the reaction site, and/or at a location proximate to the reaction site (e.g., upstream or downstream of the reaction site) such that the environmental condition within the reaction site is known and/or controlled. For example, the environmental factor may be the concentration of a gas or a dissolved gas within the reaction site or associated with the reaction site (for example, upstream or downstream of the reaction site, separated from the reaction site by a membrane, etc.). The gas may be, for example, oxygen, nitrogen, water (i.e., the relative humidity), CO₂, or the like. The environmental factor may also be a concentration of a substance in some cases. For example, the environmental factor may be an aggregate quantity, such as molarity, osmolarity, salinity, total ion concentration, pH, color, optical density, or the like. The concentration may also be the concentration of one or more compounds present within the reaction site, for example, an ion concentration such as sodium, potassium, calcium, iron or chloride ions; or a concentration of a biologically active compound, such as a protein, a lipid, or a carbohydrate source (e.g., a sugar) such as glucose, glutamine, pyruvate, apatite, an amino acid or an oligopeptide, a vitamin, a hormone, an enzyme, a protein, a growth factor, a serum, or the like. In some embodiments, the substance within the reaction site may include one or more metabolic indicators, for example, as would be found in media, or as produced as a waste products from cells. If cells are present, the sensor may also be a sensor for determining all viability, cell density, cell motility, cell differentiation, cell production (e.g., of proteins, lipids, small molecules, drugs, etc.), etc.

The environmental factor may also be a fluid property of a fluid within the reaction site, such as the pressure, the viscosity, the turbidity, the shear rate, the degree of agitation, or the flowrate of the fluid. The fluid may be, for instance, a liquid or a gas. In one set of embodiments, the environmental factor is an electrical state, for example, the charge, current, voltage, electric field strength, or resistivity or conductivity of the fluid or another substance within the reaction site. In one set of embodiments, the environmental condition is temperature or pressure. In certain embodiments, the sensor may be a ratiometric sensor, i.e., a sensor able to determine a difference or ratio between two (or more) signals, e.g., a measurement and a control signal, two measurements, etc.

Non-limiting examples of sensors useful in the invention include dye-based detection systems, affinity-based detection systems, microfabricated gravimetric analyzers, CCD cameras, optical detectors, optical microscopy systems, electrical systems, thermocouples and thermistors, pressure sensors, etc. Those of ordinary skill in the art will be able to identify other sensors for use in the invention. For example, in one set of embodiments, the chip may contain a sensor comprising one or more detectable chemicals responsive to one or more environmental factors, for example, a dye (or a combination of dyes), a fluorescent molecule, etc. One or more dyes, or fluorescent or chromogenic molecules sensitive to a specific environmental condition(s) may be chosen by those of ordinary skill in the art. Non-limiting examples of such dyes, or fluorescent or chromogenic molecules include pH-sensitive dyes such as phenol red, bromothymol blue, chlorophenol red, fluorescein, HPTS, 5(6)-carboxy-2′,7′-dimethoxyfluorescein SNARF, and phenothalein; dyes sensitive to calcium such as Fura-2 and Indo-1; dyes sensitive to chloride such as 6-methoxy-N-(3-sulfopropyl)-quinolinim and lucigenin; dyes sensitive to nitric oxide such as 4-amino-5-methylamino-2′,7′-difluorofluorescein; dyes sensitive to dissolved oxygen such as tris(4,4′-diphenyl-2,2′-bipyridine) ruthenium (II) chloride pentahydrate; dyes sensitive to dissolved CO₂; dyes sensitive to fatty acids, such as BODIPY 530-labeled glycerophosphoethanolamine; dyes sensitive to proteins such as 4-amino-4′-benzamidostilbene-2-2′-disulfonic acid (sensitive to serum albumin), X-Gal or NBT/BCIP (sensitive to certain enzymes), Tb³⁺ from TbCl₃ (sensitive to certain calcium-binding proteins), BODIPY FL phallacidin (sensitive to actin), or BOCILLIN FL (sensitive to certain penicillin-binding proteins); dyes sensitive to concentration of glucose, lactose or other components, or dyes sensitive to proteases, lactates or other metabolic byproducts, dyes sensitive to proteins, antibodies, or other cellular products, such as calcein AM, ethidium bromide, or resazurin (sensitive to viability).

In one embodiment, the dye or fluorescent molecule may be immobilized within one or more walls within the chip, e.g., within one or more walls defining the reaction site. In another embodiment, the dye or fluorescent molecule may be immobilized within a gel positioned within the chip, for example, in fluid communication with the reaction site. In yet another embodiment, the dye or fluorescent molecule may be dissolved in a media, for example, that is passed through the reaction site. The dye or fluorescent molecule may have a response generally proportional to a value of one or more environmental factors and/or other variable(s) of interest. The response may be measured, e.g., as a fluorescent signal, an absorbance signal, a wavelength or frequency, etc. A reactor and/or reaction site within a chip may be coupled to a light delivery and/or other light interacting component(s). For example, the light-interacting component may include a detection system where light (e.g., having a predetermined wavelength) arising from a dye, a fluorescent molecule, etc., may be detected and/or measured.

The sensor can include a colorimetric detection system in some cases, which may be external to the chip, or microfabricated into the chip in certain cases. In one embodiment, the colorimetric detection system can be external to the chip, but optically coupled to the reaction site, for example, using fiber optics or other light-interacting components that may be embedded in the chip (e.g., such as those described below). As an example of a colorimetric detection system, if a dye or a fluorescent molecule is used, the colorimetric detection system may be able to detect a change or shift in the frequency and/or intensity of the dye or fluorescent molecule in response to a change or shift in one or more environmental factors within a reaction site. As a specific example, Ocean Optics Inc. (Dunedin F.O.) provides fiber optic probes and spectrometers for the measurement of pH and dissolved oxygen concentration.

In some aspects of the invention, any of the above-described chips may be constructed and arranged such that the chip, or a portion thereof, such as one or more reaction sites, is able to respond to a change in an environmental condition within or associated with a reaction site, for example, by use of a control system. In some cases, each reaction site within the chip may be independently controlled in some fashion. As used herein, a “control system” is a system able to detect and/or measure one or more environmental factors within or associated with the reaction site, and cause a response or a change in the environmental conditions within or associated with the reaction site (for instance, to maintain an environmental condition at a certain value). In some cases, the control system may control the environmental factor in real time. The response produced by the control system may be based on the environmental factor in certain cases. An “active” control system, as used herein, is a system able to cause a change in an environmental factor associated with a reaction site as a direct response to a measurement of the environmental condition. The active control system may provide an agent that can be delivered, or released from the reaction, where the agent is controlled in response to a sensor able to determine a condition associated with the reaction site. A “passive” control system, as used herein, is a system able to maintain or cause a change in an environmental condition of the reaction site without requiring a measurement of an environmental factor. The passive control system may control the environmental factor within the reaction site, but not necessarily in response to a sensor or a measurement. The passive control system may allow an agent to enter or exit the reaction site without active control. For example, a passive control system may include an oxygen membrane and/or a water-permeable membrane, where the membrane can maintain the oxygen and/or the water vapor content within the reaction site, for instance, within certain predetermined limits. The control system may be able to control one or more conditions within or associated with the reaction site for any length of time, for example, 1 day, 1 week, 30 days, 60 days, 90 days, 1 year, or indefinitely in some cases.

The control system can include a number of control elements, for example, a sensor operatively connected to an actuator, and optionally to a processor. One or more of the components of the control system may be integrally connected to the chip containing the reaction site, or separate from the chip. In some cases, the control system includes components that are integral to the chip and other components that are separate from the chip. The components may be within or proximate to the reaction site (e.g., upstream or downstream of the reaction site, etc.). Of course, in some embodiments, the control system may include more than one sensor, processor, and/or actuator, depending on the application and the environmental factor(s) to be detected, measured, and/or controlled. One example of a control system is depicted in FIG. 5, in which an environmental condition 50 within chip 105, detected by a sensor 52, is transduced into a signal 51 that is transmitted to processor 54 for suitable processing. Processor 54 then produces a signal 53, which is sent to actuator 56 where the signal is converted into a response 60. In some embodiments, the control system may be able to produce a very rapid change in the environmental factor in response to a stimulus or a change in stimulus (for example, a detectable change in an environmental factor such as temperature or pH in a time of less than 5 s, less than 1 s, less than 100 ms, less than 10 ms, or less than 1 ms).

As used herein, a “processor” or a “microprocessor” is any component or device able to receive a signal from one or more sensors, store the signal, and/or convert the signal into one or more responses for one or more actuators, for example, by using a mathematical formula or an electronic or computational circuit. In one embodiment, the processor may be an expert system. The signal may be any suitable signal indicative of the environmental factor determined by the sensor, for example a pneumatic signal, an electronic signal, an optical signal, a mechanical signal, etc. Processor 54 may be any device suitable for determining a response to the signal, for example, a mechanical device or an electronic device such as a semiconductor chip. The processor may be embedded and integrally connected with the reaction site or chip, or separate from the reaction site or chip, depending on the application. In one embodiment, the processor is programmed with a process control algorithm, which can, for example, take an incoming signal from a sensor and convert the signal into a suitable output for an actuator. Any suitable algorithm(s) may be used within processor 54, for example, a PID control system, a feedback or feedforward system, a fuzzy logic control system etc. The processor may be programmed or otherwise designed to control an environmental condition within the reaction site, for example, by manipulation of an actuator.

For example, in one embodiment, processor 54 is able to maintain one or more environmental conditions (e.g., temperature or pressure) at a constant, predetermined level within a predetermined reaction site of a chip, for example, to facilitate a chemical reaction therein. In another embodiment, processor 54 is able to alter one or more environmental conditions within one or more predetermined reaction sites of a chip according to a predetermined pattern, or in response to a specific condition; for example, the processor may cause the actuator to raise the pH within a predetermined reaction site at a certain rate, the processor may cause the actuator to alter the pH of a predetermined reaction site once a specific temperature or other environmental condition has been reached, or the processor may cause the actuator to allow or prevent, or increase or decrease, the flow of a substance or an agent into a predetermined reaction site. In some embodiments, processor 54 is able to control several environmental conditions within a predetermined reaction site, for example, at least two, three, four, five, six, seven or more conditions, preferably simultaneously or nearly simultaneously depending on the application and the degree of control that is desired. For example, processor 54 may be in communication with one or more sensors and/or one or more actuators.

In certain embodiments, processor 54 may be programmed or designed to maintain one or more environmental conditions within one or more reaction sites. For example, processor 54 may be programmed or designed to maintain one or more environmental conditions within three reaction sites, within seven reaction sites, within ten reaction sites, etc. For example, where there are a plurality of reaction sites, one subset of reactions site may be held at one temperature, while a different subset of reaction sites may be held at a different temperature. As another example, one subset of reaction sites may have a first compound added thereto, while a second subset reaction sites may have a different compound added thereto. Combinations of subsets may also be used, for example, different subsets having different chemicals, temperatures, or the like. Thus, many different environmental conditions may be simultaneously controlled at different values within one chip. In some cases, the pattern of control and monitoring of the reaction sites may be altered in time, i.e., during an experiment. Thus, for instance, two reaction sites that were monitored and/or controlled simultaneously at a first point in time may be separately monitored and/or controlled at a second point in time. The control and monitoring may be preset, automated, or manually determined.

In one set of embodiments, processor 54 may be programmed or designed to maintain conditions suitable for supporting the metabolism or growth of a cell (e.g., a bacterial or a mammalian cell). For example, processor 54 may be able to control one or more of the temperature, relative humidity, pressure, oxygen concentration, CO₂ concentration, serum concentration, nutrient concentration, shear rate, or the pH within the reaction sites of the chip. Other environmental factors suitable for supporting cell growth are further described below.

As used herein, an “actuator” is a device able to affect the environment within or proximate to one or more reaction sites, or in an inlet or outlet in fluid communication with one or more reaction sites (e.g., as in channels 116 and 117 in FIG. 5A). The actuator may be separate from, or integrally connected to the reaction site or chip. For example, in some embodiments, the actuator may include a valve or a pump (including microvalves and micropumps) able to control, alter, and/or prevent the flow of a substance or agent into or out of the reaction site, for example, a chemical solution, a buffering solution (e.g., a pH buffering solution), a gas such as CO₂ or O₂, a nutrient solution, a saline solution, an acid, a base, a solution containing a carbon source, a nitrogen source, an inhibitor, a promoter, a hormone, a growth factor, an inducer, etc. The substance to be transported will depend on the specific application. In some cases, the pump may be external of the chip. As one example, the actuator may selectively open a valve that allows CO₂ or O₂ to enter the reaction site. In other cases, the pump may be internal of the chip. For example, the pump may be a piezoelectric pump or a mechanically-activated pump (e.g., activated by pressure, electrical stimulation, etc.). In one embodiment, the pump is activated by producing a gas within the chip, which may cause fluid flow within the chip; as examples, the gas may be produced by directing light such as laser light at a reactant to start a gas-producing reaction, or the gas may be produced by applying an electric current to a reactant (for instance, an electric current may be applied to water to produce gas). As another example, the actuator may include a pumping system that can create a fluid connection with a reaction site as necessary. In one particular example, a chip having a gas-permeable service may be placed in an incubator or other enclosed environment, and the atmosphere within the incubator or other environment may be controlled, thereby controlling the environmental conditions within the reaction sites.

As yet another example, the actuator may include a heating element or a cooling element, such as a heat exchanger (e.g., as shown in FIG. 2), a resistive heater or a Peltier cooler. In other embodiments, the actuator may include an electrical system, such as an electrical system that maintains a steady current, or a steady electric field gradient within the reaction site. In yet another example where at least two fluid streams enter or leave a reaction site, the actuator may include a valve or a pump that is able to control the ratio of flowrates between the two fluid streams. For instance, the actuator, in response to a signal, may act to increase an inlet flowrate and decrease an outlet flowrate within the reaction site.

In one set of embodiments, the actuator may include an energy source, such as an electromagnetic energy source, a heat source, a mechanical energy source, or an ultrasound source. In some embodiments, the electromagnetic radiation may have wavelengths or frequencies in the optical or visual range (e.g., having a wavelength of between about 400 nm and about 700 nm), infrared wavelengths (e.g., having a wavelength of between about 300 nm and 700 nm), ultraviolet wavelengths (e.g., having a wavelength of between about 400 nm and about 10 nm), or the like. In some cases, the light may cover a range of frequencies, for example, between about 350 nm and about 1000 nm, between about 300 nm and about 500 nm, between about 500 nm and about 1 nm, between about 400 nm and about 700 nm, between about 600 nm and about 1000 nm, or between about 500 nm and about 50 nm. In other cases, the light may be monochromatic (i.e., having a single frequency or a narrow frequency distribution), for example, a frequency that is commonly produced by commercial lasers, or a frequency that a fluorescent agent is excited at. For example, the frequency may be a frequency that is centered around 366 nm, 405 nm, 436 nm, 546 nm, 578 nm, 457 nm, 488 nm, 514 nm, 532 nm, 543 nm, 594 nm, 633 nm, 568 nm, or 647 nm. The monochromatic beam of light may have a narrow distribution of frequencies. For example, 90% or 95% of the frequencies may be within ±5 nm or ±3 nm of the average frequency. In certain cases, the light may be polarized (e.g., linearly or circularly), or more than one wavelength of light may be used, for example, serially or simultaneously. In some embodiments, a light-interacting component may alter the wavelength of light within the device.

In another embodiment, the actuator may be constructed and arranged to selectively kill or deactivate specific cells or types of cells, preferably without affecting nearby or adjacent cells. For example, the actuator may include an energy source directed substantially at the reaction site, or at an inlet or outlet in fluid communication with the reaction site; on detection of a specific cell or cell type by the sensor, the actuator may target the cell, for example, by directing energy at the cell, killing the cell or otherwise deactivating it in some fashion (e.g., by damaging its DNA enough to prevent replication). The energy targeted towards the cell may be any energy able to deactivate the cell, for example, electromagnetic or ionizing radiation, ultrasound, or heat energy.

In one set of embodiments, the chip is constructed and arranged to control an environmental factor associated with a reaction site by transporting an agent able to affect the environmental factor, or a precursor of an agent that is able to affect the factor, into or proximate the reaction site (i.e., such that it affects the environmental factor within the reaction site). Control of the delivery of the agent (or precursor) to the reaction site, in certain instances, may be used to control the environmental factor.

In another set of embodiments, an environmental factor within or associated with the reaction site may be altered and/or controlled without directly contacting the reaction site to an agent, e.g., an external or unsterilized agent, such as a liquid or a gas. For example, the reaction site may contain a biological specimen or a substance for use in a biological setting where sterility and/or isolation is required; or the reaction site may contain a reaction that is sensitive to, e.g., liquids or pH changes, for example, a water-sensitive reaction which must be performed in a non-humid environment, where direct contact between the agent and, the reaction site would be present difficulties.

In one set of embodiments, the chip may be constructed and arranged to allow an agent to permeate or diffuse into the reaction site. For instance, the reaction site may be defined, at least in part, by a component such as a wall or a layer of the chip, through which an agent is able to permeate. The agent may be able to alter and/or control one or more of the environmental factors within or associated with the reaction site. For instance, the component may include a membrane, such as an osmotic membrane or a semipermeable membrane (e.g., with respect to the agent) that the agent is able to permeate across. In some cases, the component may be chemically or physically inert with respect to the agent. In certain instances, a flow of agent may occur on one side of the component. In some embodiments, the flow of agent on one side of the component may occur along a meandering or non-straight pathway, for example, to increase the time of contact between the agent and the component. For example, in FIG. 2, if compartment 20 is separated from compartment 42 by a membrane (not shown) through which an agent is able to permeate, a flow of agent may occur along serpentine path 281.

In one embodiment, a chemical agent generated elsewhere within the chip may be allowed to interact with the reaction site(s) to control the environmental factor(s) therein, or one or more fluidic pathways may be created (e.g., opened) within the chip that allows an agent stored within the chip or external the chip to come into contact with the reaction site or otherwise affect the reaction site. The agent may be any agent able to alter and/or control one or more environmental factors within the reaction site. For instance, the agent may be a non-pH-neutral composition or a pH-altering agent as previously described. As an example, in FIG. 5A, chip 105 may be constructed to allow an agent to permeate and/or diffuse into the reaction site. For instance, the reaction site may include a component such as a wall (e.g., a wall of predetermined reaction site 112) or one or more layers of the chip (e.g., upper layer 100), through which an agent is able to permeate through to affect the reaction site. As another example, the component that the agent is able to penetrate in some fashion may include or be defined by a membrane, such as an osmotic membrane or a semipermeable membrane (e.g., semipermeable with respect to the agent) that the agent is able to permeate across. In some cases, the component may be chemically or physically inert with respect to the agent; for instance, the component may allow an acidic or an alkaline compound to permeate across to the reaction site without substantially damaging or altering the component. In certain instances, a flow of agent may occur on one side of the component. In some embodiments, the flow of agent on one side of the component may occur along a meandering or non-straight pathway, for example, to increase the time of contact between the agent and the component.

For instance, in the embodiment of the invention shown in FIG. 7A, chip 205 is illustrated having a predetermined reaction site 207 and a permeable upper layer 220. In this example, dispensing unit 228 is positioned proximate the reaction site such that the dispensing unit is able to produce an agent able to permeate towards and interact with reaction site 207 within a desired time frame, for example, within a few seconds or tens of seconds, minutes, or hours, depending on the application. Dispensing unit 228 may also be connected to one or more chemical sources, for example, one or more sources of gases and/or pH-altering agents, such as sources 222 and 224 as shown in the illustrative figure. As examples, source 222 may be an acid source and source 224 may be an alkaline source, source 222 and source 224 may each be acid sources or alkaline sources, source 222 may be a source of cell media and source 224 may be a source of glucose or saline, etc. FIG. 7B illustrates an expanded view of a droplet 225 containing an agent (e.g., an agent dispensed by dispensing unit 228) that has been dispensed onto the surface of chip 205 on upper layer 220. In this figure, a portion 226 of droplet 225 has partially permeated through layer 220 towards reaction site 207. Over time, permeation region 226 may expand as the agent penetrates upper layer 220 until the agent comes into contact with reaction site 207 and affecting an environment factor within the reaction site.

In some embodiments, as shown in Eq. 1, the permeability (P) of a substrate with respect to an agent (e.g., a component or a layer of the chip) may be expressed as the volumetric transfer rate of the agent (v) times the thickness (T), per area (a), time (t) and the partial pressure difference (p):
P=vT/atp (1)
The thickness of the substrate (T) may be measured in, for example, cm or mm, the time (t) in seconds, the pressure (p) in Pa, atm, cmHg, or mmHg, the area (a) in cm² or mm², and the volumetric transfer rate of the agent (v) in cm³, measured at STP (“standard temperature and pressure,” referring to a temperature of 273.15 K (0° C.) and a pressure of 101 325 Pa (1 atm)) or other standardized conditions. The permeability will thus be in units, for example, of cm³_{STP} mm/cm² s cmHg). Thus, as one, for example, the substrate may have a permeability of at least about 400×10₋₉ (cm³^{STP} cm/s cm² cmHg), at least about 500×10⁻⁹ (cm³_{STP} cm/s cm² cmHg), at least about 590×10⁻⁹ (cm³_{STP} cm/s cm² cmHg), at least about 700×10⁻⁹ (cm³_{STP} cm/s cm² cmHg), or at least about 800×10⁻⁹ (cm³_{STP} cm/s cm² cmHg) to ammonia, acetic acid, and/or CO₂. As one particular example, where the substrate is a membrane that has a thickness of rough 100 micrometers, the substrate may have a permeability of about 172 mol/day m² atm to ammonia, a permeability of about 150 mol/day m² atm to acetic acid, and/or a permeability of about 150 mol/day m² atm to CO₂.

As one example, if the environmental factor within or associated with the reaction site is pH, then the agent may be a pH-altering agent able to be delivered or transported to or proximate the reaction site to control the pH therein. As used herein, a “pH-altering” agent is any agent able to alter the pH of the environment within or associated with the reaction site, for example, an acid, a base, or an agent able to react within or proximate the reaction site to form an acid or a base. In some embodiments, the pH-altering agent is inert relative to the reaction site, and/or other component(s) of the chip. The pH-altering agent may be able to alter the pH of the environment within or associated with the reaction site to a significant or a measurable extent, for example, by at least 0.1, 0.2, 0.3, 0.4, 0.5, 0.8, 1, 2, or 3 or more pH units, depending on the required sensitivity and the specific application. The required pH sensitivity can be readily determined by those of ordinary skill in the art. For example, a chemical process that requires a change in pH to initiate a reaction may require large pH changes, while a process to regulate the pH of the reaction site near an optimum value may require sensitivity to smaller changes in pH.

As used herein, “acid” is given its ordinary definition as used in chemistry. In some cases, an acid may have a pH of less than about 7, less than 5, less than 4, less than 3, or less than 2 pH units, depending on the strength of the acid. Similarly, a “base,” or an “alkaline” is given its ordinary definition as used in the field of chemistry. In some cases, the base or alkaline may have a pH of at least about 7, at least about 8, at least about 9, at least about 11, or at least about 12 pH units. A “non-neutral” or a “non-pH-neutral” composition is a composition that is either acidic or basic (i.e., the composition has a pH that is either greater than or less than 7, preferably by a significant amount, such as by at least 1 or 2 pH units). The non-pH-neutral composition may be a solid, a liquid, or a gas in some cases. As used herein, a “gaseous” acid or base is a composition that is in the gas phase, or is generally volatile (i.e., having a high vapor pressure) and easily enters the gas phase. For example, the gaseous acid or base may have a vapor pressure of at least about 300 mmHg, at least about 400 mmHg, at least about 500 mmHg, at least about 600 mmHg, or at least about 700 mmHg. Non-limiting examples of gaseous acids include acetic acid, formic acid, propionic acid, pyruvic acid, lactic acid, SO₂, CO₂, CO, NO₂, or butyric acid; non-limiting examples of gaseous bases include ammonia, phosphine, or arsine.

In some embodiments where a component of the chip (e.g., a layer or a membrane) comprises a polymer that a molecule (e.g. a small molecule) is able to permeate, the polymer may be or include, for example, nylon, polyethylene, polypropylene, polycarbonate, polydimethylsiloxane, or copolymers or blends. In another set of embodiments, the component may include a polymer substantially impermeable to the agent being transported, but the component may be constructed or designed to allow transport of the agent to occur, for example, through a region that is porous or contains a number of channels. In yet other embodiments, the component may be impermeable to the agent being transported, but the component may be converted to a permeable form upon the addition of a permeabilizing agent. As used herein, “permeation” and “permeate” refer to any suitable non-bulk transport process. A non-bulk transport, with respect to a substrate, generally is a transport process where substantial convection or bulk flow does not occur within the substrate. For example, permeation of the agent may occur through passive diffusion, for example, through the bulk material of a component or through pores or other interstices that may exist within the component; or the transport may be facilitated or enhanced in some manner, for instance, through osmosis, electrodiffusion, electroosmosis, percolation, or through the use of a permeation-enhancing compound within the component. In some embodiments, transport of the agent may be facilitated using an externally-applied field, such as an electrical, magnetic, or a centripetal field.

In some embodiments, the component may be designed to transport an agent therethrough within a given period of time or under a certain condition. In these cases, the exact thickness, density, porosity, tortuosity, composition, or other characteristics of the component may be determinable by those of ordinary skill in the art. For example, in some cases, the diffusion of the agent across the component may be generally Fickian, and the time it takes the agent to diffuse across the component may be determined using Fick's Law. In certain cases, transport of the agent across the component may be relatively rapid, for example, in cases where a relatively thin component is used. For instance, the component may be constructed such that an agent is transported therethrough in less than about 10 minutes, less than about 5 minutes, less than about 3 minutes, or less than about 1 minute, depending on the application.

In another set of embodiments, for example, as shown in FIG. 8A, laser 230 directs laser beam 232 at compartment 235 of chip 205, for example, to activate a reaction that produces an agent able to alter an environmental factor within predetermined reaction site 207, for instance, pH or concentration. In other embodiments, of course, other forms of energy, such as heat or electrical energy, may be applied to compartment 234 (or to chip 205 in general) to activate the agent. An expanded view of FIG. 8A is shown in FIG. 8B. Laser beam 232 may be substantially directed towards compartment 235 directly from any direction or angle (as shown in FIG. 8), or indirectly, for example, through a waveguide (not shown). As shown in FIG. 8, laser beam 232 may optionally pass through one or more other layers and or components of chip 205 before reaching compartment 235 (for example, if those layers and/or components are substantially transparent). Upon absorption of the energy from laser beam 232 by agent-producing precursor(s) 237 in compartment 235, the agent-producing precursor(s) 237 may produce agent 238 in this example. Agent 238 may be, in this example, a gas such as a pH-altering gas, for example, ammonium, acetic acid, CO, CO₂, O₂, N₂, HCl, etc. Agent 238 then may permeate through at least a portion of chip 205 (for example, within a channel, or through a component and/or a layer of the chip) to interact with predetermined reaction site 207. Thus, the controlled application of light or other energy to compartment 235 may result in the alteration and/or control of an environmental factor within predetermined reaction site 207.

In some embodiments, the environmental factor within the reaction site may be altered by generating one or more agents within the chip, for example, from one or more precursors, such as precursor 237 in FIG. 8B. The agent(s) may interact with, or alter in some way, an environmental factor within the reaction site. In one embodiment, the agent may be generated within the reaction site. In another embodiment, the agent may be generated elsewhere within the chip and transported to the reaction site in some fashion, for instance, fluidically. For example, the chemical agent may be produced and/or stored within a different compartment associated with or external of the chip (e.g., as in a reservoir), then transported to the reaction site, for instance, through a channel or other fluidic connection, or by allowing it to permeate or diffuse across a membrane or another component. In one embodiment, the agent may be generated in a location proximate the reaction site, e.g., the agent may be generated in a location where it can be readily transferred or transported to the reaction site, for example, within a few seconds or tens of seconds. In another embodiment, the agent may be a gas that is transported to the reaction site, for example, through a membrane, or over a barrier that prevents liquid communication between the compartment and the reaction site, while non-gaseous products may be prevented from entering the reaction site. In certain embodiments of the invention, the reaction of the precursor(s) that produces the agent may be externally initiated. For example, a light source, such as a laser, may be applied to the precursor(s), or other energy sources such as electrical current or heat may be used to initiate a reaction of the precursor(s). In yet another embodiment, a fluidic connection may be created between the compartment and the reaction site, for example, reversibly. For instance, the fluidic connection may be created by opening a valve such as a mechanical valve or a micromechanical valve, etc. separating the compartment and the reaction site.

In some cases, additional compounds may be combined with the precursor(s) to, for example, preserve the precursor(s) against decomposition or degradation, to enhance the ability of the precursor(s) to react (e.g., a catalyst or an enzyme), or to enhance the absorption of incident energy onto the precursor(s), for instance, to increase the reaction rate of the precursor(s) to form an agent. In some embodiments, a material that is able to absorb of incident electromagnetic radiation, such as a darkened or “black” material, may be added to the precursor(s), for example, to enhance the absorption of energy. Non-limiting examples of such materials include quartz, black glass, silicon, black sand, carbon black, and the like. The additional compounds may be substantially unreactive, unable to form a transportable agent (i.e., transportable through a layer or a component of the chip), or the additional compounds may not significantly interfere with the production of the agent or with control of an environmental factor associated with the reaction site.

The agent, in certain embodiments, may be produced in a reaction that is activated at a certain temperature, such as in a thermal decomposition or degradation reaction. In some cases, the reaction to produced the agent may be initiated when the precursor(s) is exposed to at least a certain temperature able to activate the reaction, for example, a temperature of at least about 200° C., 300° C., 400° C., or 500° C. The temperature necessary to activate the reaction may be produced within the precursor(s) by any suitable technique, for example, upon the exposure of light energy, heat, electrical energy (e.g., resistive heating), an exothermic chemical reaction, or the like to the precursor(s).

In some embodiments, the agent so produced may be a gas, for example, O₂, CO, CO₂, NO, NO₂, HCl, or the like. In some cases, the agent-producing reaction may produce one or more gases and/or one or more non-gaseous products. In some cases, the gaseous agent(s) may then be transported into or proximate the reaction site (for example, through a membrane or over a barrier), while non-gaseous products (such as liquids or solids) may be prevented from entering the reaction site in some fashion.

The agent, in certain cases, may be a pH-altering agent. In some cases, the pH-altering agent may be a base, such as ammonia. The base may be generated by any suitable reaction that can generate an alkaline agent, such as through a thermal decomposition reaction of an alkaline precursor salt. For example, ammonia may be generated through the thermal decomposition of an ammonium precursor salt such as ammonium nitrate, ammonium carbonate, ammonium bicarbonate, ammonium chloride, ammonium bromide, ammonium fluoride, or the like. In other cases, the pH-altering agent may be an acid, such as acetic acid or formic acid. The acid may be generated using any suitable reaction that can generate an acidic agent, such as the thermal decomposition of an acid precursor salt. For instance, acetic acid may be produced by the thermal decomposition of sodium acetate, potassium acetate, calcium acetate, lithium acetate, magnesium acetate, or the like. Similarly, formic acid may be produced by the thermal decomposition of sodium formate, potassium formate, calcium formate, lithium formate, magnesium formate, etc. In some cases, the pH-altering agent may not be an acid or a base, but be in a form that can be converted into an acid or a base within the chip or within a reaction site. For example, the pH-altering agent may react with water to form an acid or a base within the chip or reaction site. As a non-limiting example, a gas such as CO₂ may react with water to produce carbonic acid, e.g.:
CO₂+H₂O<−>H₂CO₃<−>H⁺+HCO₃⁻

In yet another set of embodiments of the invention, the agent may be present in a compartment not in fluid communication with the reaction site; when exposure of the agent to the reaction site is desired in order to alter or control an environment factor therein is desired, a fluidic pathway may be created to enable the agent to enter into or otherwise interact with the reaction site. For example, a created fluidic pathway may be a new pathway, i.e., a non-preexisting pathway, or a pathway created in a region that did not previously contain a fluidic pathway; or the created fluidic pathway may be created in a region that previously contained a fluidic pathway that has been altered to prevent fluidic communication. In some cases, a new pathway may be created within the chip by removing or damaging a component of the chip, such as a layer, a membrane a wall defining a reaction site or a channel in fluidic communication with the reaction site, etc. As another example, the fluidic pathway may be a closed, pre-existing fluidic pathway that can be opened and/or modified under certain conditions, for instance, a valve or a switch. In one embodiment, the compartment is a sealed compartment, e.g., a compartment without access to the external environment and/or the reaction site. In another embodiment, the compartment is accessible externally (i.e., through an inlet or an outlet), but is not in fluid communication with the reaction site.

Chips of the invention may include one or more fluid pathways for delivery of species or removal of species from a reaction site. In some cases, a fluidic pathway can be created in situ (after construction of the chip, during chip setup and/or during use of the chip) by permeabilizing or damaging a component separating the compartment from the reaction site (e.g., as in a wall or a membrane), or separates the compartment from a fluidic pathway in fluid communication with the reaction site. For instance, in certain embodiments of the invention, the fluidic pathway or other means for fluidic communication may be created by permeabilizing and/or damaging (reversibly or irreversibly) a component that separates the compartment containing the agent (and/or agent precursor(s)) from fluidic communication with the reaction site, or separates the compartment from a channel or other fluidic pathway in fluid communication with the reaction site, thus creating a fluidic connection between the compartment and the reaction site. For example, the component may be permeabilized by heating the component to increase the permeability of the chemical agent or by causing the component to melt or vaporize. In some cases, the permeability of the component may be enhanced by one, two, or three or more orders of magnitude. In certain cases, the permeabilization of the component may be reversible or at least partially reversible, for example, by decreasing the temperature, or introducing a non-permeabilizing agent.

The component, in some cases, may also be damaged or otherwise altered or permeabilized through a reaction, for example, a chemical or electrochemical reaction, to produce a fluidic connection with the reaction site. For example, the component may include a metal, such as gold, silver or copper, that can be electrolyzed upon the application of a suitable electrical current. As yet another example, the component may be chemically etched, for example, with a reactive species.

In still other embodiments, the component as discussed above may be mechanically altered and/or damaged, for example, by piercing the component with a microneedle to create a fluidic pathway between the compartment and the reaction site. The microneedle or other mechanical device may originate from within the chip, or externally. In one embodiment, the component may be altered on a reversible basis, for example, the component may be self-sealing and/or comprised an elastomeric substance that can be resealed.

The component may also be damaged without the use of mechanical forces or chemicals in some cases. For example, energy may be applied to the surface to damage it. In some embodiments, the component may be ablated, for example, using heat or light. If light is used, the light may be channeled through a waveguide to the surface in some cases, or light may be applied directly to the surface.

The component may include a material able to enhance the creation of the fluidic pathway in some embodiments of the invention. As examples, the enhancing material may facilitate the absorption of light or other forms of energy, or increase the chemical reaction or transport rate. For instance, in one embodiment, the component may comprise a material that is able to absorb incident electromagnetic radiation, i.e., a darkened or “black” material, such as quartz, black glass, silicon, black sand, carbon black, and the like. As other examples, the component may include a catalyst, an enzyme, or a permeation enhancer.

In one aspect, the present invention is directed to a chip able to control gases or humidity therein. The present invention, in some embodiments, may allow humidity control to be passive and built into a chip that may be used to, for example, conduct chemical or biochemical reactions, or culture cells. In one embodiment, humidity control or maintenance may be provided to the chip in the form of a humidity controller and/or a film, optionally with low water permeability relative to the oxygen permeability. As used herein, a “humidity controller” is a device that allows certain gases, such as oxygen, carbon dioxide, or nitrogen to enter the chip, but inhibits the passage of water vapor into the chip. The humidity controller may allow passage of small amounts of water vapor into the chip, but does not allow as much water vapor to enter the chip as at least one other gas, e.g. those listed above. Examples include, but are not limited to, membranes and thin films (e.g., films having a thickness of less than 2 mm). In some embodiments, the humidity controller may be positioned as, or in, a wall of the chip, such as within a wall of a reactor unit or reaction site. In other embodiments, the humidity controller may be positioned such that it is in fluid communication with one or more reaction sites. In some embodiments, each of the reaction sites in the chip may be adjacent to, and/or in fluid communication with a humidity controller. In some cases, the humidity controller may substantially seal at least a portion of the chip.

Humidity controllers of the invention can include a humidity control material designed to maximize gas and/or minimize water vapor passage therethrough. The humidity control material of the present invention may allow the passage of certain desired gases, such as oxygen and/or carbon dioxide, while inhibiting the passage of other gases, for example, water vapor. The material of the present invention is suitable for use as a humidity controller in a chip, but is not limited to such uses; rather, the material may be used anywhere where water vapor or other specified gases are to be kept in or out, while allowing the passage of oxygen and/or other gases. For example, the humidity control material of the present invention may be useful in greenhouses or wound dressings.

In one set of embodiments, the humidity control material may include a membrane or a thin film selected to control the passage of gases and/or water vapor therethrough. In one embodiment, the humidity controller is a membrane or a thin film having a desired permeability to one or more gases. The membrane or thin film may be positioned anywhere in the chip where it is able to affect one or more reaction sites in some fashion. For example, the membrane or thin film may be positioned such that it defines the surface of one or more reaction sites.

In one set of embodiments, the membrane or thin film has a thickness of greater than about 10 micrometers, in some cases greater than about 25 micrometers, in some cases greater than about 50 micrometers, in some cases greater than about 75 micrometers, in some cases greater than about 100 micrometers, or in some cases greater than about 150 micrometers while still allowing sufficient oxygen transport therethrough, for instance, to enable cell culture to occur, as further described herein. In some cases, a membrane or a thin film having a thickness of greater than about 50 micrometers may be particularly useful, for example, during manufacturing of the chip. The membrane may have a thickness of less than 1 or 2 millimeters in some cases.

In some cases, it may be desired to incorporate the humidity control material into a structural aspect of the chip, or to incorporate structural aspects of the chip into the humidity control material. Where the humidity control material is intended to provide or supplement support, or will not itself be otherwise adequately supported, the humidity control material may also include a support layer. A support layer may comprise any material or materials that provides desired support. For example, the support layer may include one of the layers that may otherwise be included in the humidity control material for permeability, such as polydimethylsiloxane or polyfluoroorganic materials, or the support layer may comprise a different material, such as glass (for example, PYREX® glass by Corning Glass of Corning, N.Y.; or indium/tin-coated glass), latex, silicon, or the like. The support layer may be positioned anywhere within the humidity control material, for example, as an outer layer or an intermediate layer, and may be positioned to help protect one or more delicate layers. In some embodiments of the present invention, the use of a support layer may allow a large portion, or nearly all of a reaction site, reactor, or chip to be constructed of the humidity control material. Preferably, the support layer does not significantly impact the permeability of the humidity control material, or the change in permeability may be accounted for in the design of the humidity control material.

Where the chip of the present invention is intended for use with materials, such as reactants, that may damage, reduce the function, or otherwise react with or cause the humidity control material to deteriorate, the membrane may include a protection layer. The protection layer may be positioned as any component of the humidity control material, for example, as a surface layer, or interposed between a sensitive portion of the humidity control material and the material or environment that may adversely affect it. For example, the protection layer may be positioned on an inner surface of the humidity control material, particularly where the harmful material is within the chip, or on the outer surface of the humidity control material, particularly where the harmful material is outside the chip. The protection layer may also be positioned between other layers, so long as it is able to perform is protective function. Preferably, the protection layer does not significantly impact the permeability of the humidity control material, or the change in permeability may be accounted for in the design of the humidity control material.

As an example, a chip 140 including a humidity controller according to one embodiment of the present invention is illustrated in FIG. 9A. This chip includes a reaction site 142, an inlet 144, an outlet 146, and an inner wall 148. Inner wall 148 is defined on one side by a humidity controller 150. Humidity controller 150, in this embodiment, includes a membrane having a first layer 152 and a second layer 154.

Another embodiment of a chip 140 including a humidity controller is illustrated in FIG. 9A. In this embodiment, the humidity controller 150 includes a multi-layer membrane that defines a wall of a reaction chamber 142, and also defines a wall of an inlet and of an outlet. In addition to first and second layers 152 and 154, which are provided primarily for purposes of providing a desired permeability, this membrane also includes a support layer 156 positioned between first and second layers 152, 154. Other arrangements for the permeability-controlling layer(s) and support layer(s) are possible. Also provided in chip 140 in this particular example is a cell adhesion layer 158 positioned on inner wall 148 of reaction site 142, encouraging cell growth there and not in inlet 144 and outlet 146. In other embodiments, the cell adhesion layer could extend over more, or all, of the surface of humidity controller 150. It should also be appreciated that the geometry of chip 140 as illustrated in FIGS. 9A and 9B is shown by way of illustration only and that many other arrangements and chip geometry may be useful in particular embodiments.

In one set of embodiments, the humidity control material is selected to have a certain permeability and/or a certain permeance. As used herein, the “permeability” of a material is given its ordinary meaning as used in the art, i.e., an intrinsic property that generally describes the ability of a gas to pass through the material. In contrast, as used herein, the “permeance” of a material is the actual rate of gas transport through a sample of a material, i.e., an extrinsic property. The permeance of a sample of material is affected by factors such as the area or thickness of the material, the pressure differential across the material, etc. For example, in FIG. 11, the oxygen permeance of two membranes is shown to be dependent on the membrane's thickness.

A chip of the present invention, in one set of embodiments, may include a humidity control material (e.g., a membrane or a thin film) having a permeability to oxygen greater than about 3.9×10⁸ cm³/s, and in some cases greater than about 4.3×10⁻⁸ cm³/s, and/or a permeability to water vapor lower than about 1.7×10⁻⁷ cm³/s, and in some cases lower than about 1.0×10⁻⁷ cm³/s. It should be appreciated that, while control of oxygen is used as an example herein, other gases such as nitrogen or carbon dioxide may be controlled instead, at permeabilities as noted above, or a combination of gases may be controlled. It should also be appreciated that while, in the example of cells further described below, the lower limit of oxygen transfer and the upper limit of water vapor transfer may typically be desired to be controlled, in other applications, for example, in a chemical synthesis operation, it may be desired to control other parameters, for example, the upper limit of oxygen transfer and lower limit of water vapor transfer, or the lower and upper limits of other gases such as nitrogen or carbon dioxide.

The humidity control material of the present invention may be used in a wide variety of reactions and interactions. One example of a reaction is cell culture, for example to maintain a cell culture, to increase the number of available cells or cell types, or to produce a desirable cellular product. In some cases, the humidity control material may allow sufficient oxygen to enter by diffusion therethrough to support cell growth. In certain cases, the humidity control material may also be largely impermeable to microorganisms and other cells, for example to prevent contamination. Preferably, the material has low toxicity.

In embodiments where the invention is used in connection with culturing cells, cell culturing may take place over varying lengths of time, depending on the cells being cultured and other factors known to those of ordinary skill in the art. Thus, the design of the chip and the nature of the humidity control material may be adapted to the culture time. For example, the chip or humidity control material may be designed to allow it to withstand the time needed for the culture and is preferably designed to be able to be reused many times. In various embodiments, cell cultures may be performed in 24 hours, 48 hours, 1 week, 2 weeks, 4 weeks, 6 weeks, 3 months, 1 year, continuously, or any other time required for a specific cell culture.

In some cases, the humidity control material is selected to have a permeability and/or a permeance to one or more gases that corresponds to a range acceptable for culturing certain cells. For example, the humidity control material may have a permeability and/or permeance to oxygen high enough, and/or a permeability and/or permeance to water vapor low enough, to allow cell culturing. Examples of such permeabilities include the above-described permeabilities. Those of skill in the art will be able to identify specific ranges of permeabilities of certain materials appropriate for successfully culturing particular cells and cell lines, as well as larger cellular groups, such as microbial and mammalian cells, tissues, tissue engineering constructs, etc.

Thus, in one embodiment, the invention includes a method of identifying an oxygen requirement and a humidity requirement of certain cells, selecting a material having an oxygen permeability high enough to meet the oxygen requirement of the cells and a water vapor permeability low enough to meet the humidity requirement of the cells, and culturing the cells in a chip comprising a reaction site. The reaction site has at least a portion thereof formed of the selected material.

Examples of permeability ranges of a humidity control material for use in the invention, for example for use in culturing a broad range of cells, include a permeability to oxygen greater than about 100 (cm³_{STP} mm/m² atm day), and a permeability to water vapor less than about 6×10⁻⁶ (cm³_{STP} mm/m² atm day). As used herein, “STP” refers to “standard temperature and pressure,” referring to a temperature of 273.15K (0° C.) and a pressure of about 10⁵ Pa (1 atm). In another embodiment, the humidity control material may have a permeability to water that is less than about 100 (cm³_{STP} mm/m² atm day) and, in other embodiments, less than about 30 (cm³_{STP} mm/m² atm day) or less than about 10 (cm³_{STP} mm/m² atm day), and an oxygen permeability of at least about 6×10⁶ (cm³_{STP} mm/m² atm day), and in some embodiments, at least about 1×10⁷ (cm³_{STP} mm/m² atm day), and in other embodiments greater than about 3×10⁷ (cm³_{STP} mm/m² atm day) or 1×10⁸ (cm³_{STP} mm/m² atm day). Any combination of oxygen permeability and water vapor permeability listed herein can be used. For microbial cells, an example of a suitable range of oxygen permeability is provided by a membrane having a permeability to oxygen permeability greater than about 1×10³ (cm³_{STP} mm/m² atm day) and/or a permeability to water vapor is less than about 6×10⁶ (cm³_{STP} mm/m² atm day). For mammalian cells, an example suitable range is provided by a membrane of the invention having a permeability to oxygen greater than about 100 (cm³_{STP} mm/m² atm day) and a permeability to water vapor lower than about 1×10⁵ (cm³_{STP} mm/m² atm day).

For humidity control materials having a permeability to oxygen and water vapor, in certain cases, it is desired that the material have very high oxygen permeability and very low permeability to water vapor, e.g., as is indicated in FIG. 12 by “goal” region 80. For example, the material may have an oxygen permeability of greater than about 1000 (cm³_{STP} micrometer/m² day atm), in some cases greater than about 10,000 (cm³_{STP} micrometer/m² day atm), and in some cases greater than about 100,000 (cm³_{STP} micrometer/m² day atm), and/or a permeability to water vapor less than about 1000 (g micrometer/m² day), in some cases less than about 100 (g micrometer/m² day), and in some cases less than about 10 (g micrometer/m² day). For instance, as illustrated in FIG. 5, the results of materials such as high density polyethylene (“HDPE”), polyethylene terephthalate (“PET”), polypropylene (“PP”), or poly(4-methylpentene-1) (“PMP”) are shown, and these may be suitable for use with the invention, as further described below. Other materials and combinations of materials are also contemplated, e.g., as further described below.

In some embodiments, the humidity control material does not promote cell adhesion, but may include a cell adhesion layer (or a cell adhesion layer can be provided on the material) that may be any of a wide variety of hydrophilic, cytophilic, and/or biophilic materials. Examples of materials that may be suitable for a cell adhesion layer on a humidity control material include, but are not limited to, polyfluoroorganic materials, polyester, PDMS, polycarbonate, polystyrene, and aluminum oxide. As another example, the humidity control material may include a layer coated with a material that promotes cell adhesion, for example, using an RGD peptide sequence. In some embodiments, it may be desired to modify the surface of a cell adhesion layer, for example, by attachment, binding, soaking or other treatments. Example molecules that promote cell adhesion include, but are not limited to, fibronectin, laminin, albumin or collagen. Where the material includes a cell adhesion layer, the cell adhesion layer may be positioned as an inner layer or a surface layer of the membrane, or may abut an interior of the chip. Preferably, the cell adhesion layer does not significantly impact the permeability or permeance of the humidity control material, or the change in permeability or permeance may be accounted for in the design of the humidity control material.

Some of the materials used to form the humidity control material, and, in some cases, some of the layers thereof, may be selected based on the gas permeabilities of the materials, for example, as previously described. Those of ordinary skill in the art will know of methods of determining the gas permeability of a material. As one particular example method, a sample of a material having a known exposed area and thickness (e.g., a membrane) may be placed between two chambers, and a gas (or a liquid) may be placed in one chamber. The experimental time it takes for the gas (or liquid) to diffuse across the material to the other chamber and detected in a suitable fashion may then be related to the gas (or liquid) permeability of the material.

In one set of embodiments, the humidity control material may include a polymer (e.g., a single polymer type, a co-polymer, a polymer blend, a polymer derivative, etc.). Examples of polymers that may be used within the humidity control material include, but are not limited to, polyfluoroorganic materials such as polytetrafluoroethylenes (e.g., such as those marketed under the name TEFLON® by DuPont of Wilmington, DE, for example, TEFLON® AF) or certain amorphous fluoropolymers; polystyrenes; PP; silicones such as polydimethylsiloxanes; polysulfones; polycarbonates; acrylics such as polymethyl acrylate and polymethyl methacrylate; polyethylenes such as high-density polyethylenes (“HDPE”), low-density polyethylenes (“LDPE”), linear low-density polyethylenes (“LLDPE”), ultra low-density polyethylenes (“ULDPE”) etc.; PET; polyvinylchloride (“PVC”) materials, such as those marketed under the name SARAN® by Dow Chemical Co. of Midland, MI; nylons such as that marketed under the name DARTEK® by Dupont; a thermoplastic elastomer, and the like. Another example of a suitable material is a BIOFOIL® polymer membrane, made by VivaScience (Hannover, Germany). In one embodiment, the polymer may be poly(4-methylpentene-1) (“PMP”): 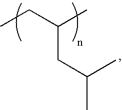
which, in some cases, may have a permeability coefficient for oxygen of about 317.2 (m³_{STP} m/s m Pa). Examples of PMPs include those marketed under the name TPX™ by Mitsui Plastics (White Plains, N.Y.). In other embodiments, the polymer may be poly(4-methylhexene-1), poly(4-methylheptene-1) poly(4-methyloctene-1), etc. In another embodiment, the polymer may be poly(1-trimethlsilyl-1-propyne) (“PTMSP”): 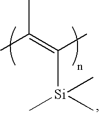
which, in some cases, may have a permeability coefficient for oxygen of about 5.78×10⁵ (cm³_{STP} mm/m² day atm). In some cases, copolymer of these and/or other polymers may be used in the humidity control material.

Of course, the first and second layers may also each include a mixture of materials in some embodiments. For example, one layer may include at least 50% by weight of one material with the balance comprising one or more other materials. In another embodiment, each layer consists essentially of a single material.

In some embodiments, the area and thickness of the humidity control material, or a layer or portion thereof, may be used to select a desired degree of permeance and/or permeability. As one example, a more water vapor-permeable material may be made thicker, or its area may be reduced, in order to reduce the amount of water vapor that reaches or leaves the area or region where humidity control is desired. In some cases, the material may be designed such that it is between about 10 micrometers and 2 mm thick. Within this range, the relative thickness of layers within multiple layers or portions of the material may vary. For example, a relatively thick layer of a polyfluoroorganic material and a relatively thin layer of vinylidene chloride may be useful in particular embodiments. As additional examples, a few micrometers of polytetrafluoroethylene may be deposited or coated onto a layer of polydimethylsiloxane, or a few micrometers of HDPE could be co-molded with PDMS.

In some cases, the polymer (or mixture of polymers) used in the humidity control material may be sufficiently hydrophobic such that the polymer is able to retain water (i.e., water vapor is not able to readily transport through the polymer). For instance, the permeability of water through a hydrophobic polymer may be less than about 1000 (g micrometer/m² day), 900 (g micrometer/m² day), 800 (g micrometer/m² day), 600 (g micrometer/m² day) or less, as previously described.

In certain embodiments, the polymer(s) used in the humidity control material may have a molecular structure open enough to readily allow the transport of oxygen therethrough. For instance, the molecular structure may allow transport of oxygen across the polymer of greater than about 1000 (cm³_{STP} micrometer/m² day atm) or more, as previously described. In one embodiment, the polymer is sufficiently branched such that the polymer is unable to form a structure under ambient conditions (e.g., a tightly crystalline structure) that limits the transport of oxygen therethrough, for instance, to less than about 1000 (cm³_{STP} micrometer/m² day atm) or 500 (cm³_{STP} micrometer/m² day atm).

In another embodiment, the polymer may include a bulky group that prevent the polymer from readily forming a structure under ambient conditions that limits the transport of oxygen therethrough. A “bulky group” on a polymer, as used herein, is a moiety sufficiently large that the polymer is unable to form a crystalline structure under ambient conditions that limits the transport of oxygen therethrough to less than about 1000 (cm³_{STP} micrometer/m² day) or 500 (cm³_{STP} micrometer/m² day). The bulky group may be, for instance, part of the backbone of the polymer or a side chain. Non-limiting examples of bulky side groups include groups containing cyclopentyl moieties, isopropyl moieties, cyclohexyl moieties, phenyl moieties, isobutyl moieties, tert-butyl moieties, cycloheptyl moieties, trimethylsilyl or other trialkylsilyl moieties etc. For example, in one set of embodiments, the polymer may have a structure: 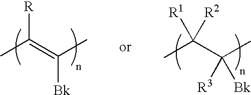
where each R independently comprises at least one atom, and Bk is a bulky group. In some cases, R may be a hydrogen or an alkyl group.

Of course, it should be understood that the polymer may have several or all of the above-described features. For example, the polymer may be a polymer blend or a copolymer that has sufficient hydrophobicity such that the polymer is able to retain water yet have a molecular structure open enough to allow sufficient oxygen permeability therethrough.

In one embodiment, the present invention achieves a permeability goal by combining two layers or portions of material. This can be achieved, for example, by including a first, more permeable layer, and a second, less permeable layer; multiple layers may also be used in other embodiments. By combining different materials and adjusting their relative thickness, a desired oxygen and water vapor permeability may be achieved. In one embodiment where the humidity control material comprises two layers or portions, they may be formed out of the same or different materials polymers. For example, the humidity control material may include a first layer including at least about 55% by weight of a first polymer or co-polymer and a second layer comprising no more than about 45% by weight of the first polymer or co-polymer. As another example, the humidity control material may include a first layer including at least about 60%, about 70%, or about 80% by weight of a first polymer or co-polymer and a second layer comprising no more than about 40%, about 30%, or about 20% by weight of the first polymer or copolymer. In some embodiments, the first polymer may comprise about 100% of the first layer and essentially none of the second layer. In some cases, at least a portion of the first layer may be co-polymerized with the second layer.

Where the humidity control material of the present invention is constructed as a membrane including two or more layers, the two or more layers may be joined in any manner that provides sufficient strength to the membranes. In some cases, the two or more layers may be sufficiently self-supporting and it may not be necessary to join the layers, meaning a space could be left therebetween if desired. In other embodiments, additional layers may be used to support the membrane. In embodiments where it is desired to join the two or more layers to provide mutual support or otherwise, examples of acceptable means of joining the layers include laminating the layers together, at least partially intermixing the layers, and co-polymerizing the layers together. Where the layers are to be intermixed, the resin that will form each layer may be partially or totally intermixed before the membrane is formed. For example, liquid pre-polymers may be mixed and then a curing agent added, or two partially cured layers can be connected with a curing agent between them, curing the layers together.

In another set of embodiments, the humidity control material of the present invention allows light to pass through it. This may allow the material to be used where light is important, for example, to facilitate a reaction such as a photocatalyzed reaction, to promote cell or plant growth, to cause a biochemical change to occur, or the like. The material may also allow observation of a region, such as a reactor or reaction site, that is protected by the humidity control material, or is located behind a humidity-controlled region. In one embodiment, the humidity control material is translucent, and, in some cases, it is at least substantially transparent. One of skill in the art will recognize that there are varying degrees of translucence and transparence, and will be able to select desired properties based upon a particular application.

The chip can include a variety of other components. For example, the chip may include components such as a light source, a flowmeter (e.g., for measuring fluid flow of a gas or a liquid), a circuit such as an integrated circuit, a reservoir (e.g., for a solution), a micromechanical or a MEMS (“microelectromechanical system”) component, a microvalve, a micropump, or the like, for example, as further described below. The components may be fabricated on the chip using techniques such as those used in standard microfabrication, similar to those used to create semiconductors (See Madou Fundamentals of Microfabrication, CRC Press, Boca Raton, Fla. 1997; and Maluf, An Introduction of Micromechanical Systems Engineering, Artech House Boston, Mass. 2000). In some embodiments, at least one, two, three or more components are integrally connected to the chip. In certain embodiments, all of the components are integrally connected to the chip.

Other examples of components suitable for use with the invention include pylon-like obstructions placed in the flow path of a stream to enhance mixing within the chip, reactor and/or reaction site, or heating, separation, and/or dispersion units within the chip, reactor and/or reaction site. For example, if a heating unit is present, the heating unit may be a miniaturized, traditional heat exchanger.

For instance, in one set of embodiments, the present invention may include a membrane, such as a membrane that may control humidity (e.g., as previously described) and/or be substantially transparent. If a membrane is present, it may be positioned anywhere in the a reactor within a chip. In one embodiment, the membrane is positioned such that it defines the surface of one or more reaction sites and/or divides a reaction site into two or more portions, which portions may have the same or different dimensions. For example, in FIG. 10A, membrane 410, which may be a humidity controller and/or be substantially transparent, defines a surface of reaction site 411. In FIG. 10B, membrane 410 defines the surface of reaction site 411 and a surface of reaction site 412. As another example, the membrane can be positioned such that it is in fluidic communication with one or more reaction sites of the chip. In some cases, the membrane may be positioned such that a pathway fluidly connecting a first reaction site with a second reaction site crosses the membrane. In another embodiment, the membrane can be positioned such that it is in fluidic communication with one or more reaction sites of the chip. In some cases, the membrane may be positioned such that a pathway fluidly connecting a first reaction site with a second reaction site crosses the membrane. For example, in FIGS. 10C and 10D, membrane 410 does not define surfaces of reaction sites 411 or 412, but is positioned such that at least one pathway fluidly connecting reaction site 411 with reaction site 412 crosses membrane 410.

As one example, in one embodiment, the membrane may be a porous membrane having, for example, a number-average pore size of greater than about 0.03 micrometers and less than about 5 micrometers. In other embodiments, the pore size of the membrane may be less than about 4 micrometers, less than about 3 micrometers, less than about 2 micrometers, less than about 1.5 micrometers, less than about 1.0 micrometers, less than about 0.75 micrometers, less than about 0.6 micrometers, less than about 0.5 micrometers, less than about 0.4 micrometers, less than about 0.3 micrometers, less than about 0.1 micrometers, less than about 0.07 micrometers, and in other embodiments, less than about 0.05 micrometers. In certain cases, the pores are also greater than 0.03 micrometers or greater than 0.08 micrometers. In some cases, the membrane may be chosen to prevent the passage of certain cells there through (e.g., bacterial cells, yeast cells, mammalian cells, etc.). For example, a membrane with a pore size of about 0.2 micrometers may prevent the passage of bacteria cells, and a membrane with a pore size of a bout 1 micrometer may prevent the passage of mammalian cells. In certain embodiments, a membrane may be chosen to prevent or permit the passage of certain molecules, e.g., micromolecules, having a certain size and/or charge, i.e., a charge and/or size selective membrane.

The membrane may be or include polymers or other materials such as polyethylene terephthalate (PET), polysulfone, polycarbonate, acrylics such as polymethyl methacrylate, polyethylene, polypropylene, regenerated cellulose, nitrocellulose, aluminum oxide, glass, fiberglass, and the like. In certain embodiments, the membrane may also be substantially transparent, e.g., as previously described. In one embodiment, for example, the membrane is a substantially transparent polyethylene terephthalate membrane having a pore size of 2 micrometers or less, for example, a ROTRAC® capillary membrane made by Oxyphen U.S.A., Inc. (New York, N.Y.).

In one set of embodiments, a chip of the invention may include a structure adapted to facilitate the reactions or interactions that are intended to take place therein (e.g., within a reaction site). For example, where a chip is intended to function as one or more bioreactors for cell culturing, the chip may include structure(s) able to improve or promote cell growth. For instance, in some cases, a surface of a reaction site may be a surface able to promote cell binding or adhesion, or the reactor and/or reaction site within the chip may include a structure that includes a cell adhesion layer, which may include any of a wide variety of hydrophilic, cytophilic, and/or biophilic materials. As examples, the surface may be ionized, coated (e.g., with a support material) and/or micropatterned with any of a wide variety of hydrophilic, cytophilic, and/or biophilic materials, for example, materials having exposed carboxylic acid, alcohol, and/or amino groups. Examples of materials that may be suitable for a cell adhesion layer include, but are not limited to, polyfluoroorganic materials, polyester, PDMS, polycarbonate, polystyrene, and aluminum oxide. As another example, the structure may include a layer coated with a material that promotes cell adhesion, for example, an RGD peptide sequence, or the structure may be treated in such a way that it is able to promote cell adhesion, for example, the surface may be treated such that the surface becomes relatively more hydrophilic, cytophilic, and/or biophilic. In some embodiments, it may be desired to modify the surface of a cell adhesion layer, for instance with materials that promote cell adhesion, for example, by attachment, binding, soaking or other treatments. Example materials that promote cell adhesion include, but are not limited to, fibronectin, laminin, albumin or collagen. In other embodiments, for example, where certain types of bacteria or anchorage-independent cells are used, the surface may be formed out of a hydrophobic, cytophobic, and/or biophobic material, or the surface may be treated in some fashion to make it more hydrophobic, cytophobic, and/or biophobic, for example, by using aliphatic hydrocarbons and/or fluorocarbons.

In some embodiments, the chip may include a “light-interacting component,” i.e., a component that interacts with light, for example, by producing light, reacting to light, causing a change in a property of light, directing light, altering light, etc. As used herein, a “light-interacting component” is a component that interacts with light in some fashion related to chip and/or reactor function, for example, by producing light, reacting to light, causing a change in a property of light, directing light, altering light, etc., in a manner that affects a sample within a chip or reactor and/or determines something about the sample (the presence of the sample, a characteristic of the sample, etc.). In one embodiment, the component produces light, such as in a light-emitting diode (“LED”) or a laser. In another embodiment, the light-interacting component may be a component that is sensitive to light or responds to light, such as a photodetector or a photovoltaic cell. In yet another embodiment, the light-interacting component may manipulate or alter light in some fashion, for example, by focusing or collimating light, or causing light to diverge, such as in a lens, or spectrally dispersing light, such as in a diffraction grating or a prism. In another embodiment, the light-interacting component may be able to transmit or redirect the direction of light in some fashion, such as along a bent path or around a corner, for example, as in a waveguide or mirror. In yet another embodiment, the light-interacting component may alter a property of light incident on the component, such as the degree of polarization or the frequency, for example, as in a polarizer or an interferometer. Other devices, or combinations of devices, are also possible. In general, the term “light-interacting component” does not encompass components or devices that passively transmit light without significant modification, alteration, or redirection, such as air, or a plane of glass or plastic (e.g., a “window”). The term “light-interacting component” also does not generally encompass components that passively absorb essentially all incident light without a response, such as would be found in an opaque material.

In embodiments in which a light-interacting component is provided in conjunction with a reactor, it may be positioned anywhere on or within the reactor. For example, the light-interacting component may be placed within or adjacent to a reaction site. In some cases, the light-interacting component is integrally connected with the reaction site, for example, as a wall or a surface of the reaction site.

As another example, the light-interacting component may be positioned elsewhere in, or integrally connected to, the chip, such that at least a portion of light entering the light-interacting component is in optical communication with the reaction site. As used herein, the term “optical communication” generally refers to any pathway that provides for the transport of electromagnetic radiation, such as visible light. Optical communication includes direct, “line-of-sight” communication. Optical communication may also be facilitated, for example, by the use of optical devices such as lenses, filters, optical fiber, waveguides, diffraction gratings, mirrors, beamsplitters, prisms, and the like. In some embodiments, the light-interacting component may direct light to or from more than one reaction site, or the light-interacting component may direct light from more than one light source to a reaction site. In certain embodiments, more than one light-interacting component may be present.

The light-interacting component may include a waveguide in some cases. The term “waveguide,” as used herein, is given its ordinary meaning in the art and may include optical fibers. A waveguide is generally able to receive light and guide or transmit a portion of that light to a destination not within “line-of-sight” communication (although a waveguide can transmit light to a line-of-sight region), e.g., around bends, corners, and similar obstacles without substantial losses.

In some embodiments, a waveguide may include a “core” region of material embedded or surrounded, at least in part, by a second “cladding” material, which may have a lower refractive index. The core may have any shape, for example, a slab, a strip, or a cylinder of material.

The waveguide, or at least a portion of the waveguide, may be fashioned out of any material able to transmit or light to or from the reaction site. The waveguide may be substantially transparent, or translucent in some cases. In some embodiments, the waveguide may be formed out of a silicon-based material, for example, glass, ion-implanted glass, quartz, silicon, silicon oxide, silicon nitride, silicon carbide, polysilicon, coated glass, conductive glass, indium-tin-oxide glass and the like. In other embodiments, the waveguide may comprise other transparent or translucent organic or inorganic materials. For example, in certain embodiments, the waveguide may comprise a polymer including, but not limited to, polyacrylate, polymethacrylate, polycarbonate, polystyrene, polypropylene, polyethylene, polyimide, polyvinylidene fluoride, an ion-exchanged polymer, and fluorinated derivatives of the above. Combinations, blends, or copolymers are also possible.

In one embodiment, the waveguide or a portion thereof may be surrounded by or coated with a highly reflective material, for example, silver or aluminum. In another embodiment, the waveguide may be fashioned such that it comprises a central material (e.g., a core) having a first index of refraction, and a surrounding material (e.g., a cladding) having a second index of refraction. The cladding may have an index of refraction that is less than the index of refraction of the central material. In yet another embodiment, the index of refraction of the core or the cladding may vary over the cross section. As one example, the core may be a graded index optical fiber, where the index of refraction is generally highest near the center of the core.

Under these conditions, a substantial portion of the light traveling through the central material may be internally reflected (“total internal reflection”) as a result of this refractive index difference. Electromagnetic radiation entering one end of the waveguide may be confined to the central region due to the phenomenon of total internal reflection at the core-cladding boundary. The light may be transported through the core, without significant absorption by the cladding material or other surrounding materials, until it reaches the end of the waveguide, or a predetermined region of the waveguide that light is allowed to exit from. Light traveling through the central material may be directed around corners and other obstacles without a significant loss of intensity, for example, with an attenuation coefficient of less than about 10 db/cm or 20 db/cm. In another embodiment, the waveguide may have more than one central material or more than one surrounding material.

As one example of a waveguide, both the central and surrounding materials forming the waveguide may each be a glass. As another example, a waveguide may be formed out of a polymer and a silicon-based material, such that the material with the lower index of refraction surrounds the material with the higher index of refraction. As yet another example, the waveguide may be constructed out of a single material surrounded by, for example, air or a portion of the chip having a higher index of refraction than the waveguide, thus resulting in a condition where total internal reflection may occur at the waveguide/air or waveguide/chip interface.

The waveguide may be constructed by any suitable technique known to those of ordinary skill in the art, for example, by milling, grinding, or machining (e.g., by cutting or etching a channel into a chip substrate, then depositing material into the channel, optionally using a sealant). The waveguide may also be formed, for example, by depositing layers of materials during the chip fabrication process. The deposited material, in some cases, can have a higher index of refraction than the surrounding reactor substrate, thus forming a general core-cladding structure, as previously described. The waveguide may also be constructed by laser etching of materials forming the chip, such as glass or plastic, in such a way as to manipulate/alter the refractive index, relative to the surrounding material. In some cases, the refractive index of the etched/non-etched portion may be controlled so as to create a core-cladding structure.

In some embodiments, the light-interacting component may be, or include, a source of light. The light source may be any light source in optical communication with the reaction site. For example, the light source may be external or ambient light, a coherent or monochromatic beam of light such as created in an LED, or a laser such as a semiconductor laser or a quantum well laser. The light source may be integrally connected with a portion of the chip, for example, in a laser diode fabricated as part of the chip, or the light source may be separate from the chip and not integrally connected with it, but still positioned so as to allow optical communication with the reaction site. The light source may produce a single wavelength or a substantially monochromatic wavelength, or a wide range of wavelengths, as previously described. The source of light, in certain embodiments, may also be generated in a chemical reaction or a biological process, such as a chemical reaction that produces photons, for example, a reaction involving GFP (“green fluorescence protein”) or luciferase, or through fluorescence or phosphorescence. For example, incident electrons, electrical current, friction, heat, chemical or biological reactions may be applied to generate light, for example, within a sample located within a reaction site, or from a reaction center located within the chip in optical communication with the reaction site.

In certain cases, the light-interacting component may include a filter, for example, a low pass filter, a high pass filter, a notch filter, a spatial filter, a wavelength-selecting filter, or the like. The filter may be able to, for example, substantially reduce or eliminate a portion of the incident light. For example, the filter may eliminate or substantially reduce light having a wavelength below about 350 nm or greater than about 1000 nm. In another embodiment, the filter may be able to reduce noise within the incident light, or increase the signal-to-noise ratio of the incident light. In still another embodiment, the filter may be able to polarize the incident light, for example, linearly or circularly.

In some embodiments, the light-interacting component may include an optical element in optical communication with the reaction site. As used herein, an “optical element” refers to any element or device able to alter the pathway of light entering or exiting the optical element, for example, by focusing or collimating the light, or causing the light to diverge. For example, the optical element may focus the incident light to a single point or a small region, or the optical element may collimate or redirect divergent beams of light to form a parallel or converging beams of light. The term “focus” generally refers to the ability to cause rays of light to converge to a point or a small region. The term “collimate” generally refers to the ability to increase the convergence of rays of light, not necessarily to a point or a small region, for example, such that the beam focuses at an infinite distance. As one example, diverging beams of light may be collimated into parallel beams of light. In certain embodiments, the optical element may disperse or cause light to diverge, for example, as in a diverging lens. In other embodiments, the optical element may be, for example, a beamsplitter, an optical coating (e.g., a dichroic, an antireflective, or a reflective coating), an optical grating, a diffraction grating, or the like.

In one set of embodiments, the optical element may be a lens. The lens may be any lens, such as a converging or a diverging lens. The lens may be, for example, a meniscus, a plano-convex lens, a plano-concave lens, a double convex lens, a double concave lens, a Fresnel lens, a spherical lens, an aspheric lens, a binary lens, or the like. The optical element may also be a mirror, such as a planar mirror, a curved mirror, a parabolic mirror, or the like. In other embodiments, the optical element may cause light to disperse, for example, as in a diffraction grating or a prism.

In certain cases, a material having a different index of refraction may be used. For example, in embodiments in which light reaches the optical element through a waveguide, the optical element may be a material having a different index of refraction than the waveguide. In some cases, the index of refraction of the optical element will be about the same as or more than the index of refraction of the waveguide.

In some cases, a material having a graded index of refraction (a “GRIN” material) may be used as an optical element. The GRIN material may minimize the amount of divergence inherent in light reaching the GRIN material. For example, a material of uniform thickness can be made to act as a lens by varying its refractive index along a cross section of the element. In one embodiment, the GRIN material may redirect divergent rays of light into a parallel arrangement. In another embodiment, the GRIN material does not necessarily have a uniform thickness, and a combination of the graded index of refraction of the material and the shape of the material may be used to focus or collimate the light.

The light-interacting component, in some embodiments, may include a component that is able to convert light to electricity, such as a photosensor or photodetector, a photomultiplier, a photocell, a photodiode such as an avalanche photodiode, a photodiode array, a CCD chip (“charge-coupled device”) or the like. The component may be used, in some cases, to determine the state or condition of a substance within a reaction site, for example, through emission (including fluorescence or phosphorescence), absorbance, scattering, optical density, polarization measurements, or other measurements, including using the human eye.

In other cases, the light-interacting component may be used for imaging purposes, for example, to image a portion of a cell or other material located at or near the reaction site, or to determine whether a cell has adhered to a surface.

In some cases, the light-interacting component may be used to produce electricity. In one embodiment, a photocell may be integrally fabricated within the chip using one or more layers comprising semiconductor materials.

In some embodiments, light may be directed to the reaction site, for example, to activate or inhibit a chemical reaction. For example, a reaction may require the use of light for activation, or a light-sensitive enzyme may be inhibited by applying light to the enzyme. In certain embodiments, light directed to the reaction site may be used as a probe or a signal source. The light may be delivered in a controlled manner to the reaction site in certain embodiments, for example, so that the light reaching the reaction site has a specific wavelength, polarization, or intensity.

In some embodiments, a portion of the light arising from the reaction site may be detected and analyzed. The light arising from the reaction site may be reflected or refracted light, for example, light directed to the reaction as previously described, or the light may be produced through physical means, for example, through fluorescence or phosphorescence. In certain embodiments, the light may be generated within the reaction site, as previously described. Light from the reaction site may be analyzed using any suitable analytical technique, for example, infrared spectroscopy, FTIR (“Fourier Transform Infrared Spectroscopy”), Raman spectroscopy, absorption spectroscopy, fluorescence spectroscopy, optical density, circular dichroism, light scattering, polarimetry, refractometry, turbidity measurements, quasielectric light scattering, or any other suitable techniques. In another embodiment, imaging of the reaction site may be performed, for example using optical imaging, or infrared imaging.

In some embodiments of the invention, a reactor and/or a reaction site within a chip may be constructed and arranged to maintain an environment that promotes the growth of one or more types of living cells, for example, simultaneously. In some cases, the reaction site may be provided with fluid flow, oxygen, nutrient distribution, etc., conditions that are similar to those found in living tissue, for example, tissue that the cells originate from. Thus, the chip may be able to provide conditions that are closer to in vivo than those provided by batch culture systems. In embodiments where one or more cells are used in the reaction site, the cells may be any cell or cell type, for instance a prokaryotic cell or a eukaryotic cell. For example, the cell may be a bacterium or other single-cell organism, a plant cell, an insect cell, a fungi cell or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell. In some embodiments, more than one cell type may be used simultaneously, for example, fibroblasts and hepatocytes. In certain embodiments, cell monolayers, tissue cultures or cellular constructs (e.g., cells located on a non-living scaffold), and the like may also be used in the reaction site. The precise environmental conditions necessary in the reaction site for a specific cell type or types may be determined by those of ordinary skill in the art.

In some instances, the cells may produce chemical or biological compounds of therapeutic and/or diagnostic interest, for instance, in nanogram, microgram, milligram or gram or higher quantities. For example, the cells may be able to produce products such as monoclonal antibodies, proteins such as recombinant proteins, amino acids, hormones, vitamins, drug or pharmaceuticals, other therapeutic molecules, artificial chemicals, polymers, tracers such as GFP (“green fluorescent protein”) or luciferase, etc. In one set of embodiments, the cells may be used for drug discovery and/or drug developmental purposes. For instance, the cells may be exposed to an agent suspected of interacting with the cells. Non-limiting examples of such agents include a carcinogenic or mutagenic compound, a synthetic compound, a hormone or hormone analog, a vitamin, a tracer, a drug or a pharmaceutical, a virus, a prion, a bacteria, etc. For example, in one embodiment, the invention may be used in automating cell culture to enable high-throughput processing of monoclonal antibodies and/or other compounds of interest. In another embodiment, the invention may be used to screen cells, cell types, cell growth conditions, or the like, for example, to determine self viability, self production rates, etc. In some cases, the invention may be used in high through put screening techniques. For example, the invention may be used to assess the effect of one or more selected compounds on cell growth, normal or abnormal biological function of a cell or cell type, expression of a protein or other agent produced by the cell, or the like. The invention may also be used to investigate the effects of various environmental factors on cell growth, cell biological function, production of a cell product, etc.

In certain cases, a reactor and/or a reaction site within a chip may be constructed and arranged to prevent, facilitate, and/or determine a chemical or a biochemical reaction with the living cells within the reaction site (for example, to determine the effect, if any, of an agent such as a drug, a hormone, a vitamin, an antibiotic, an enzyme, an antibody, a protein, a carbohydrate, etc. on a living cell). For example, one or more agents suspected of being able to interact with a cell may be added to a reactor and/or a reaction site containing the cell, and the response of the cell to the agent(s) may be determined, using the systems and methods of the invention.

In some cases, the cells may be sensitive to light. For example, the cell may be a plant cell that responds to a light stimulus or is photosynthetic. In another embodiment, the light may be used to grow cells, such as mammalian cells sensitive to light, or plant cells. In yet another embodiment, the cell may be a bacterium that is attracted to or is repelled by light. In another embodiment, the cell may be an animal cell having a light receptor or other light-signaling response, for example, a rod cell or a cone cell. In yet another embodiment, the cell may be a genetically engineered cell having a light receptor or another light-sensitive molecule, for example, one that decomposes or forms reactive entities upon exposure to light, or stimulates a biological process to occur. In other cases, the cell may be insensitive to light; light applied to the chip may be used for analysis of the cells, for example, detection, imaging, counting, morphological analysis, or spectroscopic analysis. In still other cases, the light may be used to kill the cells, for example, directly, or by inducing an apoptotic reaction.

In some embodiments, the chip may be constructed and arranged such that cells within the chip can be maintained in a metabolically active state, for example, such that the cells are able to grow and divide. For instance, the chip may be constructed such that one or more additional surfaces can be added to the reaction site, for example, as in a series of plates, or the chip may be constructed such that the cells are able to divide while remaining attached to a substrate. In some cases, the chip may be constructed such that cells may be harvested or removed from the chip, for example, through an outlet of the chip, or by removal of a surface from the reaction site, optionally without substantially disturbing other cells present within the chip. The chip may be able to maintain the cells in a metabolically active state for any suitable length of time, for example, 1 day, 1 week, 30 days, 60 days, 90 days, 1 year, or indefinitely in some cases.

In one aspect, the present invention provides any of the above-mentioned chips packaged in kits, optionally including instructions for use of the chips. That is, the kit can include a description of use of the chip, for example, for use with a microplate, or an apparatus adapted to handle microplates. As used herein, “instructions” can define a component of instruction and/or promotion, and typically involve written instructions on or associated with packaging of the invention. Instructions also can include any oral or electronic instructions provided in any manner such that a user of the chip will clearly recognize that the instructions are to be associated with the chip. Additionally, the kit may include other components depending on the specific application, for example, containers, adapters, syringes, needles, replacement parts, etc. As used herein, “promoted” includes all methods of doing business including methods of education, hospital and other clinical instruction, scientific inquiry, drug discovery or development, academic research, pharmaceutical industry activity including pharmaceutical sales, and any advertising or other promotional activity including written, oral and electronic communication of any form, associated with the invention.

The function and advantage of these and other embodiments of the present invention will be more fully understood from the examples below. The following examples are intended to illustrate the benefits of the present invention, but do not exemplify the full scope of the invention.

### EXAMPLE 1

In this example, a chip, as illustrated generally in FIG. 5A, was prepared in accordance with an embodiment of the invention.

A first chip layer having associated fluidic channels, ports, chambers, other reaction sites, etc. therein was injection molded or machined from a stock sheet of acrylic or polycarbonate. This first layer was attached to a machined or injection molded flat bottom plate (also acrylic or polycarbonate) by means of a pressure-sensitive silicone adhesive (Dielectric Polymers). A 0.2 micrometer pore size membrane (Osmonics, Minnetonka, Minn.) was also attached to the top side of the first layer by means of the pressure-sensitive silicone adhesive.

A second chip layer (including chamber top) having associated fluidic channels, ports, chambers, other reaction sites, etc. therein was cast in a mold using PDMS. This second layer was fashioned to be alignable with the first chip layer. The second layer was aligned with the chambers in the first chip layer and attached by means of the pressure-sensitive silicone adhesive, forming a completed chip. The PDMS top could function as a septum or a self-sealing membrane by itself, or in some cases, an additional partial layer of PDMS could be bonded over an inlet or outlet of the chip using the pressure-sensitive adhesive.

### EXAMPLE 2

In this example, an embodiment of this experiment was used to demonstrate pH sensing.

Several chips similar to the one described in Example 1 were prepared. Each chip included a predetermined reaction site as defined by a chamber within the chip. The chamber depth of the bottom chamber (i.e., the distance of the chamber from the surface of the chip) was about 3 mm.

Fourteen solutions of 0.1 M phosphate buffer (K₂HPO₄/KH₂PO₄, both from Sigma-Aldrich, Milwaukee, Wis.) having differing pH were prepared with 5 micromolar solution of CDMF. CDMF (5(6)-carboxy-2′,7′-dimethoxyfluorescein; Helix Research, Springfield, Oreg.) is a fluorescent pH dye. A series of reaction sites on three different chips were each filled with the CDMF solutions.

The fluorescent intensity (“I”) of the CDMF solutions in each chamber within each chip was measured upon excitation at two wavelengths, 510 nm and 450 nm. The light sources used for excitation were high intensity light-emitting diodes (LEDs, LXHL-BE01 and -BR₀₂; Lumileds, San Jose, Calif.). The LED light was placed in optical communication with a 600 micron diameter optical fiber (P600-2, Ocean Optics) by a lens (74-UV, Ocean Optics), then directed to the chip. The emitted light was collected by a 25.4 mm f-1 lens (Thorlabs, Newton, N.J.) and optically communicated to another 600 micron fiber which, in turn, was in optical communication with a computer-controlled spectrophotometer (USB-2000F, Ocean Optics). The emission intensity reported in both cases was measured at 560 nm.

Sample results from these experiments are shown in FIG. 13 where the ratio of intensities was plotted versus the solution pH. Intensities were measured at 560 nm upon excitation by 450 nm light (I₄₅₀ nm) and 510 nm light (I₅₁₀ nm), and the ratio of these values was plotted as (I₄₅₀ nm/I₅₁₀ nm). The response of the fluorescent signal was found to correlate well with pH over the range of at least about 6 to at least about 8.

Thus, this experiment demonstrating the capability of optically addressing one embodiment of the invention to measure and control pH using ratiometric fluorescence techniques.

### EXAMPLE 3

This example illustrates the preparation of a chip in accordance with an embodiment of the invention.

A chip layer having associated fluidic channels, ports, chambers, etc. therein was cast in polydimethylsiloxane (PDMS, Sylgard 184, Dow Corning, Midland, MI) using a machined aluminum mold. The PDMS layer was cured at 90° C. for 20 minutes. The PDMS layer was attached to a bottom plate by means of a pressure sensitive silicone adhesive layer (Dielectric Polymers, Holyoke, Mass.). The bottom plate was made of acrylic or polycarbonate and was machined from sheet stock or injection molded. The layers were bonded by compressing the layers in a hydraulic press (Carver, Wabash, Ind.), forming the completed chip. The PDMS top could function as a septum itself, or in some cases, an additional partial layer of PDMS could be bonded over an inlet or outlet of the chip using the pressure sensitive silicone adhesive.

### EXAMPLE 4

This example illustrates the control of the pH within a reaction site of a chip, according to another embodiment of the invention.

Multiple chips similar to the one described in Example 3 were prepared using PDMS, having a geometry similar to the embodiment illustrated in FIG. 5. Each chip included three predetermined reaction site defined by a chamber within the chip. The chamber depth (distance from the surface of the chip) was 500 microns.

Three chambers of one chip were each filled with a solution of 50 micromolar chlorophenol red dye (Sigma-Aldrich, Milwaukee, Wis.). Cholorphenol red is known to undergo a color change from yellow to purple as the solution gets more basic (i.e., as the pH of the solution increases). This color change can be monitored by measuring the absorbance of the solution at a wavelength of 574 nm.

The pH of the reaction sites within the chips was determined optically. The light source (tungsten halogen; LH-1; Ocean Optics) was connected to an optical fiber (P100-2; Ocean Optics) which terminated with a collimating lens (74-UV; Ocean Optics) (these components are not shown on FIG. 15). The optical fiber assembly delivered light 310 to the reaction site 320. The transmitted light 315, now at least partially attenuated by the turbidity of sample 325 within reaction site 320, was collected with another collimating lens/fiber assembly (not shown) which transmitted it to a computer-controlled spectrophotometer 330 (USB-2000; Ocean Optics) The optical density (“OD”) was calculated as OD=log(I/I₀).

To control the pH within predetermined reaction site 320, a small amount (about 20 microliters) of ammonia solution (Sigma-Aldrich, Milwaukee, Wis.) was placed on top of the chip, generally proximate reaction site 320. The light absorbance at 574 nm of the reaction site was monitored over the course of two hours. Three concentrations of ammonia were used, as shown in FIG. 14: 4.0 M NH₄OH (●), 1.5 M NH₄OH (▪) and a control, water (▾). In FIG. 14, the optical density at 574 nm was plotted versus time for the three solutions, using 50 micromolar chlorophenol red as the pH indicator. Initial and final pH values were estimated from the observed change in OD.

It was found that volatile ammonia was able to permeate PDMS and enter the reaction site, thereby substantially increasing the pH of the solution within the reaction site through gaseous non-liquid transport through the PDMS, i.e., without making direct liquid contact to the liquid within the predetermined reaction site. It was also demonstrated that both the total change in pH and the rate of change within the predetermined reaction site could independently be controlled by adjusting the concentration of ammonia. By adjusting the thickness of the cover and the permeability of the cover material, the rate of pH change within the reaction site was also controlled.

Similar results, where the pH was controllably lowered instead of raised, were also demonstrated using methods similar to those described above. In those experiments, acetic acid was used as the pH-altering agent.

### EXAMPLE 5

This example illustrates an embodiment of the invention as used to adjust the pH within a predetermined reaction site while avoiding any liquid contact therein.

A microreactor was constructed out of polydimethylsiloxane (PDMS). This particular device had a footprint of 127.77 mm by 85.48 mm, generally the same size as a 96 microwell plate. This particular device was assembled by combining the various layers of materials, membranes, and barrier/interface layers to form a stacked composite structure having a 200 microliter chamber, as described in Example 1.

The pH of the chamber was monitored using a pH-altering agent, chlorophenol red, within the cell culture chamber. The emission spectra of the chamber was recorded every 10 seconds for about 90 minutes. At an initial time, a drop of ammonia (20 microliters, 4.0 M) was placed on a thin layer of PDMS covering the chamber. The ammonia gas was allowed to diffuse as a gas across the PDMS to enter the chamber, thus illustrating gaseous non-liquid transport of an agent to the predetermined reaction site.

A plot of the optical density of the chamber with respect to time of this experiment is shown in FIG. 16, for wavelengths of 480 nm, 574 nm, and 700 nm. A wavelength of 480 nm is indicative of the agent chlorophenol red, with higher optical density values indicating more alkaline conditions. These data show a rapid increase in the optical density at 574 nm over a period of about 3 minutes, beginning at about 5 minutes, indicating a rapid change in pH to more alkaline conditions during the experiment. In this experiment, the pH in the chamber was observed to rapidly increase from an initial value of 4.35 to a final value of 10.5.

Thus, this example illustrates the controlled alteration of the pH of a chamber without directly contacting the chamber with a liquid.

### EXAMPLE 6

This example illustrates the ratiometric determination of the pH within a reaction site of a chip according to an embodiment of the invention.

A chip was prepared using methods similar to those in Example 1. A pH sensor for the chip was constructed by immobilizing a fluorescent, pH-sensitive dye in a gel. The gel was prepared as follows. A stock solution of 15 ml tetraethoxysilane (TEOS) and 20 ml ethanol (both from Sigma-Aldrich, Milwaukee, Wis.) was prepared and kept sealed until use. To make the sol/gel, 1 ml of the TEOS solution was mixed with 1 ml of 500 micromolar solution of carboxyfluorescein (Sigma-Aldrich) in a 1:1 solution of ethanol and water. To this mixture, 0.1 ml of 0.5 M hydrochloric acid (Sigma-Aldrich) was added to catalyze formation of the sol/gel. Aliquots of 20 microliters of the catalyzed mixture were pipetted into small wells (500 microns deep) in the bottom plate. The plates with the sol/gel mixture were then allowed to gel over 48 hours in a humid environment. After the gel completely cured, the carboxyfluorescein dye was immobilized on the bottom plate.

The gel was placed in fluidic contact within the reaction site. Solutions having known pH values were added into the reaction site. The fluorescence of the gel in contract with the reaction site, indicative of the pH within the reaction site, was monitored using a ratiometric fluorescent procedure. In this procedure, the fluorescent response of the pH-sensitive dye at two different wavelengths (510 and 480 nm) in response to the pH was determined using a commercially-available UV-visible spectrometer. By using solutions having different known pH's within the reaction site, the ratio of the response at 510 nm and the response of 480 nm was shown to be proportional to the pH of the solution, thus demonstrating ratiometric determination of the pH within a reaction site.

### EXAMPLE 7

In this example, control of the pH within a reaction site of a chip was demonstrated according to one embodiment of the invention.

A chip similar to the one described in Example 1 was attached to a control system. A computer was used to record the pH values determined using the ratiometric procedure described above, and, using a control algorithm, the computer was able to determine whether control action to adjust the pH within the reaction site was necessary. When the computer determined that a control action was required, a fluidic connection was established between the chip and an external pumping system by opening a valve that connected the chip to the external pumping system. The external pumping system was then allowed to add an amount of an acid (e.g., ammonium hydroxide) or a base (e.g., acetic acid) to adjust the pH of the fluid within the reaction site to the required set-point. The amount of acid or base to be added was determined by the computer using the control algorithm.

### EXAMPLE 8

In this example, control of the pH within the reaction site was demonstrated in accordance with another embodiment of the invention.

A chip similar to the one described in Example 1 was attached to a control system. A fluorescent, pH-sensitive dye was immobilized in a gel in accordance with Example 6, and a computer was connected to the chip, similar to the method described in Example 7. When the computer determined that a control action was required to adjust the pH within the reaction site, the computer caused a fluidic system to dose a determined amount of ammonium hydroxide (base) or acetic acid (acid) on a permeable membrane in fluid communication with the reaction site. Control of the pH was then achieved by the action of acid or base diffusing through the membrane to enter the reaction site.

### EXAMPLE 9

This example illustrates various chips of the invention formed from multiple layers of dissimilar materials. A variety of adhesives were used to fix the interface layers to the rigid cell culture or sealing layers depending on the materials involved. One adhesive used for bonding PDMS to polycarbonate was a two-part urethane epoxy mixed with un-cured PDMS. The adhesive process used to bond rigid polycarbonate layers to each other was either sonic welding or a heated press. The reaction site was designed to be about 200 microns thick and had a volume of roughly 20 microliters.

In this example, a chip 280 having reaction site 240 was fabricated. As shown in FIG. 17A, a polycarbonate layer 244 was attached to PDMS layer 242. A gap within PDMS layer 242 defined reaction site 240 when the chip was assembled, as shown in FIG. 17A. PDMS layer 242 was attached to polycarbonate layer 244 using the above-described two-part urethane epoxy mixed with un-cured PDMS.

A similar chip is illustrated in FIG. 17B. In this figure, reaction site 240 was defined by layer 245, which was a thin, rigid layer of polycarbonate. Between layers 242 and 245 was a gas-permeable film 246 (BIOFOIL® made by VivaScience). Layers 244, 245, 246 and 242 of chip 80 were joined using the above-described adhesive processes.

### EXAMPLE 10

This example illustrates various chips of the invention formed from multiple layers of dissimilar materials. A variety of adhesives were used to fix the interface layers to the rigid cell culture or sealing layers depending on the materials involved. One adhesive used for bonding PDMS to polycarbonate was a two-part urethane epoxy mixed with un-cured PDMS. The adhesive process used to bond rigid polycarbonate layers to each other was either sonic welding or a heated press. The reaction site was designed to be about 200 microns thick and had a volume of roughly 20 microliters.

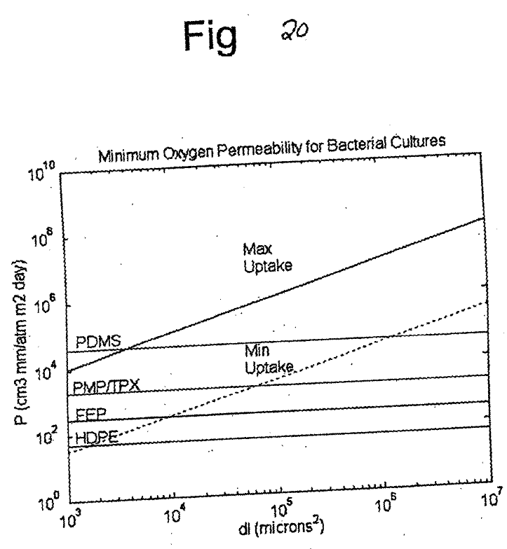

The fabrication of the chips illustrated in FIGS. 18A and 18B were similar to those described in Example 9, including the adhesion methods. In FIG. 18A, the reservoir layer 248 was fashioned from polycarbonate and was positioned between gas-permeable film 246 (BIOFOIL®) and polycarbonate layer 244. Reservoir layer 248 has a gap (i.e., a hole or a partially hollowed out space) that defines reaction site 50, which was a reservoir in this example. In FIG. 18A, the reaction site 240 was defined by a gap interface layer 242.

In FIG. 18B, polycarbonate layer 248 was used to define reaction site 250. Additionally, a second gas-permeable membrane 249 (BIOFOIL®) was used between polycarbonate layer 245 (defining reaction site 240) and polycarbonate layer 248.

### EXAMPLE 11

This example illustrates the fabrication of an embodiment of the invention without using adhesive materials. The reaction site was designed to be about 200 microns thick and had a volume of roughly 20 microliters.

The layout of this example, illustrated in FIG. 19, is similar to that illustrated in FIG. 18B of Example 10, except that an additional compression layer 252 was used to mechanically hold the other layers in place. No adhesive materials were used in this example. Instead, screws 253 extending from polycarbonate layer 252 through the other layers of the chip were secured to layer 244 to fabricate chip 280.

### EXAMPLE 12

In this example, an embodiment of the present invention is illustrated as used in a chip sealed by a membrane having a permeability to oxygen high enough to allow culture of living cells. The amount of oxygen required in this example is a function of the number of cells present and the oxygen requirements for the cells' metabolism. This is illustrated in the equations 2-4 below.
V=Ad (2)

$$\begin{matrix}P = \frac{nrdl}{{p}_{in} - {p}_{out}} & ( 3 ) \\ \frac{PA ( {p}_{in} - {p}_{out} )}{l} = \frac{Δ {m}_{gas}}{Δ t} = nrV & ( 4 )\end{matrix}$$

In these equations, P represents the permeability (typically measured in units of cm³_{STP} mm/m² atm day), A is the area (typically measured in m²), pᵢₙ is the oxygen partial pressure in the chip (typically measured in atm), pₒᵤₜ is the oxygen partial pressure outside the chip (typically measured in atm), l is the membrane thickness (typically measured in micrometers), V is the volume of the chip (typically measured in microliters), d is the cell culture chamber depth (typically measured in micrometers), n is the cell density (typically measured in cell/ml), and r is the specific oxygen demand per cell (typically measured in O₂/cell h).

Equation 4 represents a mass balance equating oxygen consumed by the growing culture to that available via diffusion through the film. Equation 2 sets the volume of the culture chamber equal to cross sectional area of the membrane contacting the chamber equal area out of both sides. Rearrangement yields Equation 3, thus expressing the minimum oxygen permeability needed to sustain cells of a given population density and metabolic rate as a function of film thickness and chamber depth

Values for P generally depend on the polymer and the permeant system, and were varied in this example for oxygen between 39,000 (cm³_{STP} mm/m² atm day) for silicon to 0.01 (cm³_{STP} mm/m² atm day) for EVA; pᵢₙ, was varied between 0.05 atm and 0.2 atm, and pₒᵤₜ was assumed to be 0.2 atm. The film thickness, 1, was varied between 1 micrometer and 2 mm. Vwas held to be less than 1 ml, and the cell culture depth, d, ranged between 30 micrometers and 2 mm. The cell density, n, was assumed in this example to be between 10⁵ cells/ml and 10⁷ cells/ml for mammalian cells and between 10⁹ cells/ml and 10₁₁ cells/ml for bacteria. The specific oxygen demand per cell ranged between 0.5 and 5×10⁻¹² mol O₂/cell h.

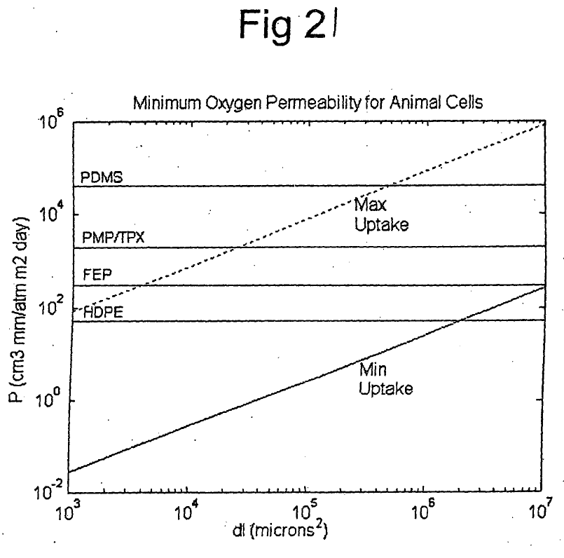

Equations 2-4 were then used to generate FIGS. 20 and FIG. 21. FIG. 20 is a graph of oxygen permeability requirements for bacterial cell culture as a function of film thickness and device geometry. FIG. 21 is a graph of oxygen permeability requirements for bacterial cell culture as a function of film thickness and device geometry. In both figures, flat horizontal lines represent the permeability of likely membrane or thin film construction materials, while diagonal lines represent the highest and lowest expected oxygen requirement. In these figures, n, the cell density, and r, the specific reaction rate, were set to the highest and lowest values, and the partial pressure differential (pᵢₙ−pₒᵤₜ) was set to 0.05 atm. The required permeability was then linear in the product of d, the chip depth and l, the thickness of the covering film.

### EXAMPLE 13

This example illustrates the use of an embodiment of the invention to determine the turbidity of a solution. This example generally corresponds to the common practice of measuring cell density of bacterial cells by nephelometry (light scattering measured at 90° to the primary beam). See, generally, Methods for General Bacteriology, P. Gerhardt, Ed., 1981 Washington D.C. p. 197.

A chip having an integrated waveguide was constructed as follows. The top layer of the chip was prepared and cast with polydimethylsiloxane (PDMS, Sylgard 184, Dow Corning, Midland, Mich.) using a machined aluminum mold.

A short section of polymeric waveguide (500 microns square, acrylic; South Coast Fiber, Alachua, Fla.) was laid in the machined aluminum mold such that one end abutted the edge of the mold and the other end extended to the edge of the mold. Fluid PDMS was poured into the mold and allowed to cure. The PDMS was cured at 90° C. for 20 minutes, immobilizing the waveguide in the chip and creating a light path from the edge of the chip to a predetermined reaction site, a chamber. The cured PDMS layer was adhered to a flat polystyrene bottom layer, forming the completed chip (the PDMS layer spontaneously adhered to the polystyrene layer). The depth of the chamber form the surface of the chip was about 1 mm.

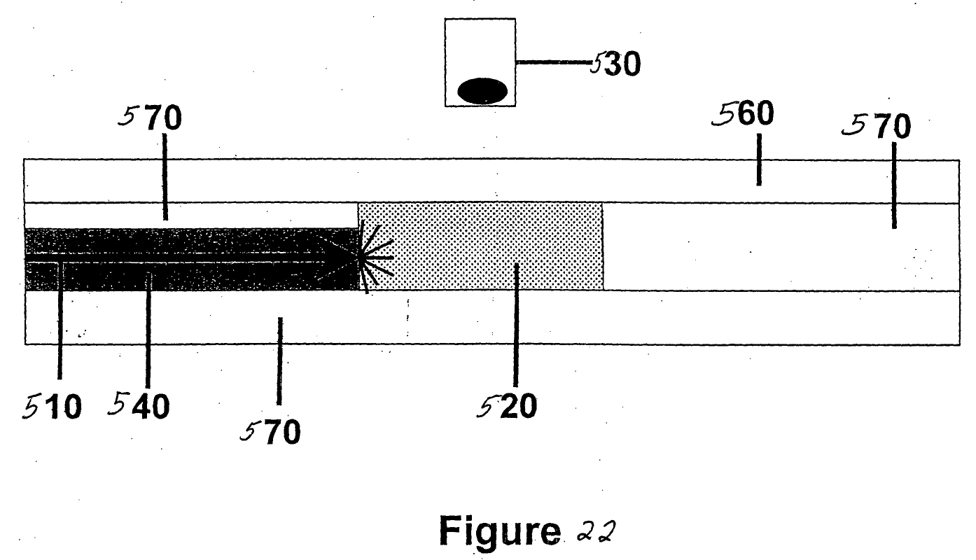

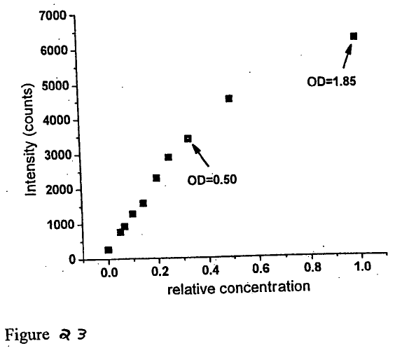

Light scattering was measured from a series of turbid solutions contained in the above chip. With reference to FIG. 22, the output of a helium-neon laser (05-LHP-991, wavelength=632.8 nm; Melles Griot Lasers, Carlsbad, Calif.) was focused onto the end of waveguide 540 which transmitted the light to the reaction site 520. The detector 530 consisted of a collimating lens (74-UV f/2 lens; Ocean Optics, Dunedin, Fla.), an optical fiber (P600-2, 600 micron dia.; Ocean Optics), and an attached spectrophotometer (USB-2000F; Ocean Optics). The detection angle was ˜90° from the axis of the waveguide.

The reaction site was filled with a series of turbid solutions of non-dairy coffee creamer (Sugar Foods, New York, N.Y.) which had absorbance values at 632 nm ranging from 0.05 to 1.85. A plot of scattered light intensity (632 nm) vs. relative concentration is given in FIG. 23. Linear correlation was observed for the solutions with optical density values ranging from 0.05 to 0.5. At higher concentrations, the scattered light response became non-linear.

### EXAMPLE 14

This example demonstrates an optically addressable reaction site, in accordance with an embodiment of the invention.

A chip was prepared using methods similar to those in Example 13. The chips used in this experiment were generally prepared. The distance of the reaction site from the surface of the chip was about 200 microns. As discussed below, the chip was optically addressed to measure optical density, using an arrangement similar to that pictured in FIG. 15.

The light source (tungsten halogen, LH-1; Ocean Optics) was connected to an optical fiber (P100-2; Ocean Optics) which terminated with a collimating lens (74-UV; Ocean Optics) (not shown in FIG. 15). The optical fiber assembly delivered light 310 to a reaction site 320. The transmitted light 315, now at least partially attenuated by the turbidity of sample 325, was collected with another collimating lens/fiber assembly (not shown) which in turn transmitted to detector 330, a computer-controlled spectrophotometer (USB-2000; Ocean Optics). The optical density was calculated as OD=log(I/I₀).

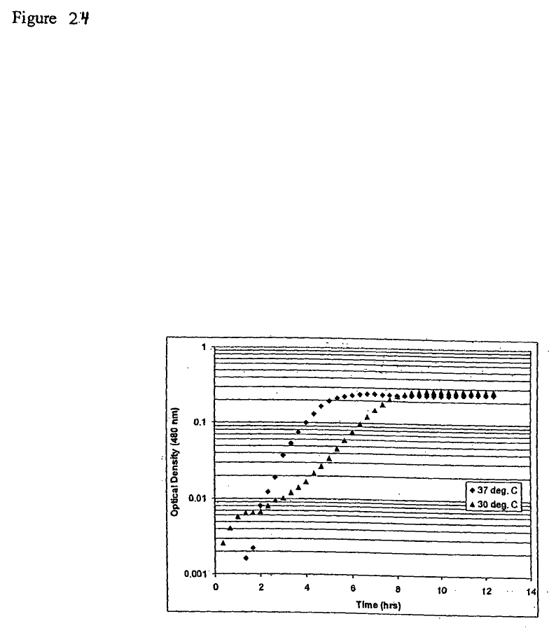

The optical density (“OD”) of a bacterial culture (E. coli BL21 in chemically defined media w/glucose) was monitored over a 13 hour growth period in a reaction site. The results from this experiment are shown in FIG. 24, which illustrates the growth of E. coli BL21 at 30° C. and 37° C. in the reaction sites of the chip, as monitored by a fiber optic spectrometer. These data thus demonstrate the validity of measuring cell growth by optically addressing reactions of the invention.

### EXAMPLE 15

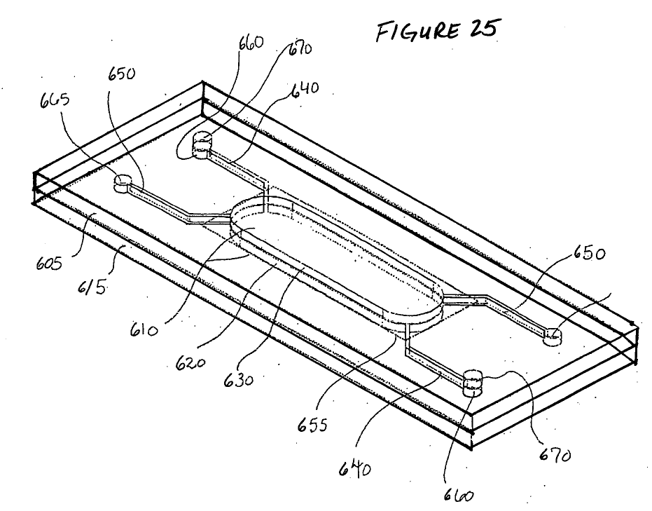

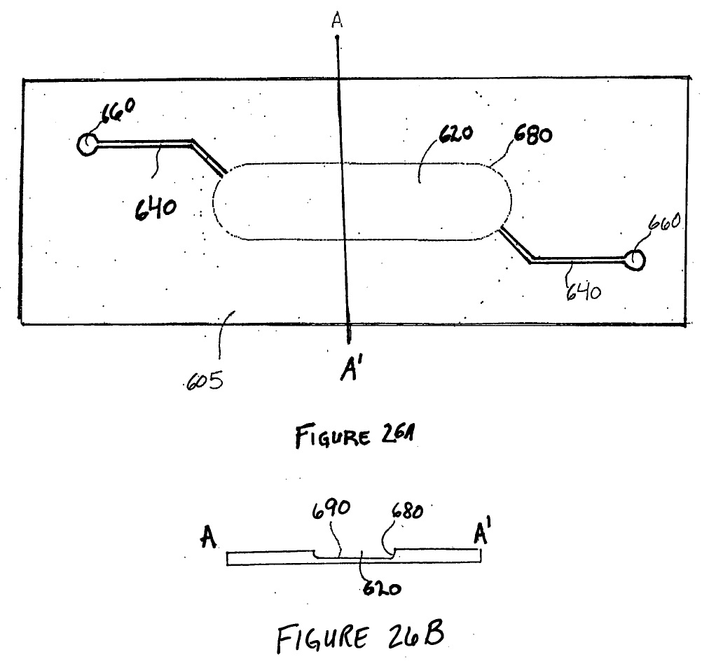

FIG. 25 is a perspective view of a planar solid substrate having a single reaction site (e.g., a chamber) and various channels. The planar substrate comprises two separately molded silicone sheets 605 and 615. In this embodiment, reaction site 610 and channels 640 and 650 are formed by juxtaposing elements molded into silicone sheets 605 and 615.

Chamber 610 in FIG. 25 includes a lower cell culture portion 620 and an upper reservoir portion 630. The lower cell culture portion 620 is in fluid communication with two lower portion channels 640 located at opposite corners of the lower cell culture portion 620. The upper reservoir portion 630 is in fluid communication with two upper portion channels 650 located at opposite corners of the upper reservoir portion 630. The upper reservoir portion 630 and its associated upper portion channels 650 are molded into upper silicone sheet 605, while the lower cell culture portion 620 and its associated lower portion channels 640 are molded into lower silicone sheet 615. The upper reservoir portion 630 and the lower cell culture portion 620 are separated by a membrane 655 that extends beyond chamber 610 between the upper silicone sheet 605 and the lower silicone sheet 615. The membrane, in this example, is substantially impermeable to mammalian cells, but is permeable to proteins, small molecules, and the like. Of course, in other embodiments, other impermeable or semipermeable membranes may be used, for example, a humidity control membrane.

Each of the upper portion channels 650, in FIG. 25, ends at an upper portion port 665 that passes completely through upper silicone sheet 615. This arrangement allows the upper portion port to be connected to additional channels, supply chambers, waste chambers, product chambers and the like that are connected at the upper surface of the upper silicone sheet. Of course, access to the upper portion channels can be provided in other ways.

In FIG. 25, each of the lower portion channels 640 ends at a lower portion port 660 that passes upward through the lower silicone sheet 605. Each lower portion port 660 is aligned with an opening 670 in upper silicone sheet 615. This arrangement allows access to each lower portion port through the upper silicone sheet 615 and allows each lower portion port to be connected to additional channels, supply chambers, waste chambers, product chambers and the like that are connected at the upper surface of the upper silicone sheet. Of course, access to the lower portion channels can be provided in other ways.

As noted above, the upper reservoir portion of the chamber and its associated channels are molded directly into the upper silicone sheet while the lower cell culture portion of the chamber and its associated channels are separately molded into the lower silicone sheet. Thus, prior to assembly of the apparatus as shown in FIG. 25, each silicone sheet includes an open upper or lower portion of the chamber and several open channels. A completely enclosed two-portion chamber and enclosed channels are formed by sandwiching a selectively permeable membrane between opposed upper and lower silicone sheets. In the embodiment of FIG. 25, the lower silicone sheet serves to close the open upper portion channels and the upper portion silicone sheet serves to close the open lower portion channels. As shown in FIG. 25, the membrane can extend beyond the walls of the chamber so that it lies between the upper and lower silicone sheets. The two silicone sheets are held together using any convenient fixture.

The silicone into which the portions of the chamber and channels are molded is sufficiently gas permeable to provide adequate gas exchange for the growth of aerobic cells in the chamber of the device.

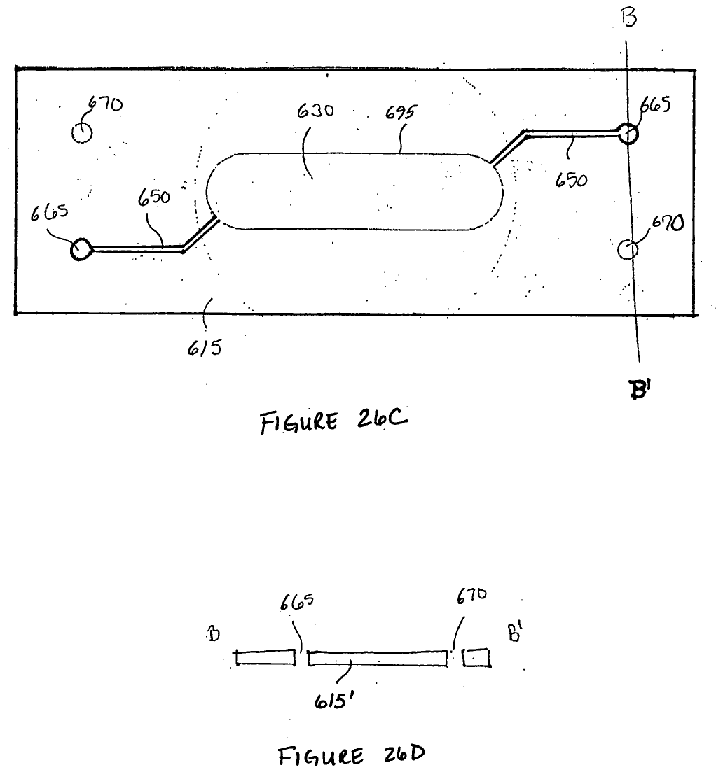

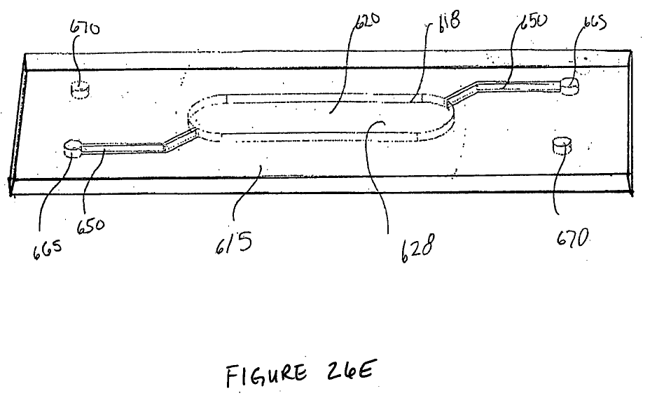

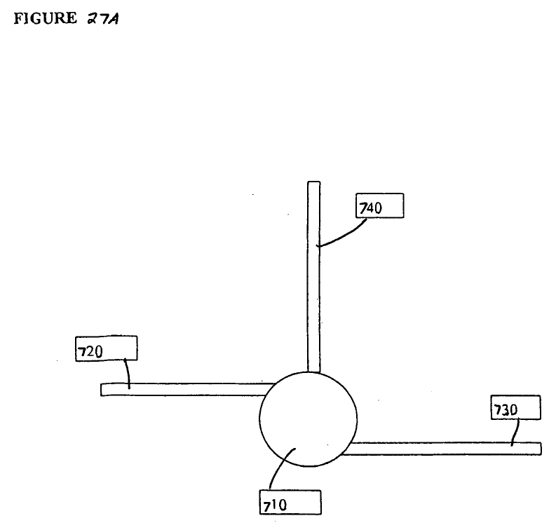

FIG. 26A is a plan view of the lower silicone sheet 605 showing the lower cell culture portion 620 of the chamber along with its associated channels 640 and lower portion ports 660. The wall 680 of lower cell culture portion 620 lacks abrupt transitions and corners. This facilitates complete mixing and dispersion of material introduced into the lower cell culture portion.

FIG. 26B is a cross-section of lower silicone sheet 605 along A-A′ in FIG. 2. The base 690 of the lower cell culture portion 620 is substantially planar and perpendicular to the wall 680 of the lower cell culture portion 620. In this embodiment, base 690 curves gently upward to meet the wall 680. This absence of sharp corners, in this example, facilitates complete mixing and dispersion of material in the lower cell culture portion 620.

FIG. 26C is a plan view of upper silicone sheet 615 showing the upper reservoir portion 630 of the chamber along with its associated channels 650, both of which end at an upper portion port 665 that provides access through the upper silicone sheet 615 to the upper portion channels. The wall 695 of upper reservoir portion 630 lacks abrupt transitions and corners in this example. This facilitates complete mixing and dispersion of material introduced into the upper reservoir portion 630. In the assembled device, passages 670 in the upper silicone sheets 615 are aligned with the lower portion ports the lower silicone sheet, allowing access to the lower portion channels through the upper silicone sheet.

FIG. 26D is a cross-section of upper silicone sheet 615 along B-B′ in FIG. 26C. As can be seen in this view, passage 670 provides an opening through the upper silicone sheet 615. This opening is aligned with one of the lower portion ports when the upper silicone sheet and the lower silicone sheet are joined to form a complete chamber. Upper portion port 665 is molded into upper silicone sheet 615 and provides access to the upper portion channels.

FIG. 26E is a perspective view of the upper reservoir portion of the chamber along with associated channels. The upper reservoir portion 620 and associated channels 650 are molded into an upper silicone sheet 615. The base 628 of the upper reservoir portion 620 is planar in this example. In this embodiment, the wall of the upper reservoir portion 618 is perpendicular to the base 628 of the upper portion. The base 628 can curve gently upward to meet the wall 618 in order to facilitate mixing and dispersion of material in the upper portion. The upper portion ports 665 located at the ends of the channels 650 allow the introduction of material into the channels. The upper silicone sheet 615 includes two passages 670 that permit access to the lower portion ports when the upper silicone sheet and lower silicone sheet are joined to form a complete chamber.

### EXAMPLE 16

In this example, a device was fabricated using three layers. In this embodiment, the bottom layer is a solid slab. The middle layer has a membrane molded into it that separates an upper reservoir portion from a lower cell culture portion, both of which are molded into the middle layer. The upper reservoir portion and the upper portion microchannels are molded into the upper surface of the middle layer and the lower cell culture portion and the lower portion microchannels are molded into the lower surface of the middle layer. Openings passing through the middle layer permit access to the lower portion microchannels. The top layer has four openings passing through it to serve as ports for the four microchannels. The top layer serves to seal the upper reservoir portion and its associated microchannels, while allowing access to all ports. The bottom layer serves to seal the lower cell culture portion and its associated microchannels.

### EXAMPLE 17

In this prophetic example, a fluidic device of the invention is used to examine the effect of chemical agent A on fermentation of a bacterium. Twelve fluidics, each bearing a single chamber having a cell culture portion and reservoir portion are aligned in parallel. The fluidics are sterilized and sterile growth media is pumped into each cell culture portion through a fluid delivery system. The reservoir portions of six fluidics receive a measured aliquot of chemical agent A and growth medium through the fluid delivery system and the remaining six receive growth medium only. Having six fluidics for each case provides a measure of redundancy for statistical purposes. The cell culture portion of each of the 12 fluidics is inoculated with a volume of concentrated cells, the volume being about 1/20 to 1/10 the volume of the cell culture portion. The growth of the microorganisms is monitored in each of the 12 fluidics by measuring pH, dissolved oxygen concentration, and cell density through the use of appropriate sensors in the fluidics. The fluidic heat exchangers, addition of chemicals, and airflow rate, the fluidic can control temperature, pH, and dissolved oxygen concentration, respectively. When cells reach stationary phase, the average cell growth rate and average final cell concentration are computed for the six fluidics with chemical agent A and for the six fluidics without. By comparing these averages, chemical agent A can be said to enhance cell growth, have no significant effect, or hinder cell growth.

### EXAMPLE 18

In this prophetic example, a fluidic device of the invention is used to provide an environment in which to grow cells or tissue that closely resembles that found in humans or mammals. With respect to drug screening, the fluidic device can monitor responses of cells to a drug candidate. These responses can includes increase or decrease in cell growth rate, cell metabolic changes, cell physiological changes, or changes in uptake or release of biological molecules. With many fluidics operating in parallel, different cell lines can be tested along with screening multiple drug candidates or various drug combinations. By incorporating necessary electronics and software to monitor and control an array of fluidics, the screening process can be automated.

Twenty fluidics each containing a single chamber divided into a cell culture portion and a reservoir portion are sterilized. Sterile animal cell culture media is pumped into the cell culture portion of each of the chambers through the fluid delivery system. Each fluidic is then inoculated with mammalian cells that are genetically engineered to produce a therapeutic protein. The cells are allowed to grow to production stage all the while their growth and environment is monitored by sensors in the fluidic. The fluidic, through control of temperature, pH, and air flow rate, is able to maintain an optimal environment for growth of the cells. Once at production stage, the fluidics are separated into four groups of five. Three of the four groups receive various cocktails of inducers for the therapeutic protein while the fourth group serves as a control and thus receives no inducers. The inducers and control sample are introduced into the reservoir portions of chambers through the fluid delivery system. A marker chemical that binds with the therapeutic protein is introduced along with inducers. When the culture is irradiated with light at a wavelength that excites the bound marker chemical, the chemical then fluoresces, and the intensity of fluorescence is proportional to the concentration of therapeutic protein in the culture. Both the irradiated light and the fluorescent signal are passed through the detection window covering the fluidic chamber. The fluorescent signal is picked up by a photodetector outside the fluidic. Production of the therapeutic protein is monitored for each of the four groups, and at the end of production, average production rates and average total production can be computed for each group. Comparison of production between the four groups can then determine the effectiveness of the various inducers on protein production.

### EXAMPLE 19

In this prophetic example, a fluidic device is used in an adsorption assay, for example, to model the adsorption of drugs and others agents in the gut. For example, the fluidic device can be provided with a chamber divided into two portions by a polycarbonate membrane having a 3.0, 2.0, or 1.0 micron pore size. Caco-2 (colon carcinoma cells) are grown on one surface of the membrane within a first portion of the chamber until they are differentiated. A drug or other agent is introduced into the portion of the chamber containing the cells. Passage of the drug or other agents through the cell layer into a second portion of the chamber is monitored.

A similar arrangement can be used for a cell migration assay. In such an assay, a membrane with a 5.0-12.0 micron pore size is used.

### EXAMPLE 20

Useful quantities of a large number of target proteins are produced as follows in this prophetic example.

A microfabricated bioreactor containing one or more cell growth chambers is sterilized and sterile growth media is pumped into each growth chamber through a fluid delivery system. For convenience, the bioreactor can contain, for example, 96 cell growth chambers arranged in the same manner as the wells of a 96 well plate. Each chamber receives an aliquot of mammalian cells and an aliquot of DNA encoding proteins of interest and, optionally, one or more selectable marks. The cells are transfected with the added DNA by calcium phosphate transfection or some other technique.

After transfection is complete, each chamber contains cells that express a different protein of interest. The cells are cultured so as to produce useful quantities of the proteins of interest which can then be harvested and analyzed or passed through microchannels to be analyzed using the microreactor system described above.

As an alternative, the cells can be transfected with the DNA molecules of interest prior to introduction into the growth chambers.

### EXAMPLE 21

Useful quantities of a large number of target proteins are produced as follows in this prophetic example.

A microfabricated bioreactor containing one or more cell growth chamber is sterilized and sterile growth media is pumped into each growth chamber through a fluid delivery system. Each chamber receives an aliquot of mammalian cells and an aliquot of a mixture of DNA molecules encoding proteins of interest and, optionally, one or more selectable markers. The cells are transfected with the added DNA by calcium phosphate transfection or some other technique.

After transfection is complete, each chamber contains cells that express one or more of the different proteins of interest. The cells are cultured so as to produce useful quantities of the proteins of interest which can then be harvested and analyzed or passed through microchannels to be analyzed using the microreactor system described above.

### EXAMPLE 22

In this prophetic example, useful quantities of a large number of target proteins are produced as follows.

A microfabricated bioreactor is sterilized and sterile growth media is pumped into each growth chamber through a fluid delivery system. Each chamber receives an aliquot of mammalian cells. A different agent is added to each chamber or each chamber is incubated under different conditions. As a result of the differing treatments, the cells in each chamber potentially produce a different group of proteins.

The cells are cultured so as to produce useful quantities of the proteins of interest which can then be harvested and analyzed or passed through microchannels to be analyzed using the microreactor system described above.

### EXAMPLE 23

In this prophetic example, useful quantities of a large number of target proteins are produced as follows.

A microfabricated bioreactor containing one or more cell growth chambers is sterilized and sterile growth media is pumped into each growth chamber through a fluid delivery system. Each chamber receives an aliquot of mammalian cells and an aliquot of DNA or a mixture of DNA molecules encoding proteins of interest and, optionally, one or more selectable markers. The cells are transfected with the added DNA by calcium phosphate transfection or some other technique.

After transfection is complete, the cells in each chamber are genetically mutated by the action of ionizing radiation, ultraviolet light, or other physical, chemical or biological mutagenesis agents. After genetic mutation, each chamber contains cells that express one or more of the different proteins of interest at potentially different rates and under different gene expression profiles. The cells are cultured so as to produce useful quantities of the proteins of interest which can then be harvested and analyzed or passed through microchannels to be analyzed using the microreactor system described above.

### EXAMPLE 24

Useful quantities of a large number of target proteins can also be produced as follows in this prophetic example.

A microfabricated bioreactor containing one or more cell growth chambers is sterilized and sterile growth media is pumped into each growth chamber through a fluid delivery system. Each chamber receives an aliquot of bacterial or fungal cells and an aliquot of a mixture of DNA molecules encoding proteins of interest within a genetic vector.

After genetic modification of the cells is complete, each chamber contains cells that express one or more of the different proteins of interest. The cells are cultured so as to produce useful quantities of the proteins of interest which can then be harvested and analyzed or passed through microchannels to be analyzed using the microreactor system described above.

### EXAMPLE 25

In this prophetic example, useful quantities of a large number of target proteins can also be produced as follows.

A microfabricated bioreactor containing one or more cell growth chambers is sterilized and sterile growth media is pumped into each growth chamber through a fluid delivery system. Each chamber is implanted with a tissue sample displaying a phenotype of interest.

The tissue samples are incubated so as to produce useful quantities of the proteins of interest which can then be harvested and analyzed or passed through microchannels to be analyzed using the microreactor system described above.

### EXAMPLE 26

This example illustrates the construction of certain embodiments of the invention.

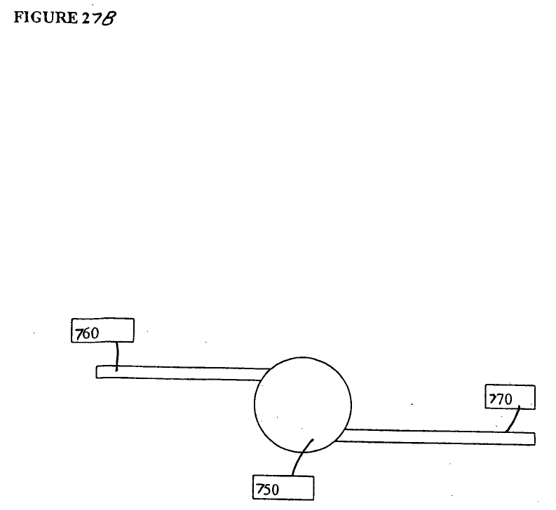

FIG. 27A depicts a cross-sectional view of the cell growth chamber of a microfabricated bioreactor device useful in the methods of the invention. The cell growth chamber 710 is a cylinder about 7 mm in diameter and about 0.1 mm in height having a total volume of 3.85 microliters. The chamber is fluidly connected to three microchannels. The first microchannel 720 is 0.4 mm wide by 0.1 mm deep and serves as a liquid inlet. The second microchannel 730 has similar dimension and serves as a liquid outlet. The third microchannel 740 is 0.2 mm wide by 0.1 mm deep. This microchannel can be used to introduce cells or any desired material into the chamber. The three microchannels and the cell growth chamber are etched into a solid support material.

FIG. 27B depicts a cross-sectional view of a gas headspace portion associated with a cell growth chamber. This allows a continuous supply of air to pass through the microfabricated bioreactor. A cylindrical chamber 750 that is about 7 mm in diameter and about 0.05 mm in height is etched in glass along with a gas inlet microchannel 760 and gas outlet microchannel 770, both of which are about 0.05 mm wide by about 0.05 mm deep. The cylindrical chamber of the gas headspace portion is matched over the cell growth chamber. The two halves can then be bonded together so as to form a tight seal.

To prevent the air flowing through the gas headspace from removing liquid in the cell growth chamber in this example, a membrane can be placed in so as to separate the gas headspace from the liquid filled bioreactor. The membrane retards passage of water and allows for the passage of air. The various microchannels are connected to supply units or waste units. These units as well as mixing devices, control valves, pumps, sensors, and monitoring devices can be integrated into the substrate in which the cell growth chamber is built or can be externally provided. The entire assembly can be placed above or below a heat exchanger (or sandwiched between two heat exchangers) to control the temperature of the unit.

The silicone into which the portions of the chamber and microchannels are molded, in this particular example, is sufficiently gas permeable to provide adequate gas exchange for the growth of aerobic cells in the chamber of the device.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An apparatus, comprising:
a chip comprising a predetermined reaction site having an inlet, an outlet, and a volume of less than about 1 ml, the predetermined reaction site constructed and arranged to maintain at least one living cell at the predetermined reaction site,
wherein the chip is constructed and arranged to stably connect in a predetermined, aligned relationship to other, similar chips.

2. The apparatus of claim 1, wherein the chip is enclosed.

3. The apparatus of claim 2, wherein the chip has an evaporation rate of less than about 100 microliters per day.

4. The apparatus of claim 3, wherein the chip has an evaporation rate of less than about 50 microliters per day.

5. The apparatus of claim 4, wherein the chip has an evaporation rate of less than about 20 microliters per day.

6. The apparatus of claim 1, wherein the chip has a length of about 128 mm.

7. The apparatus of claim 1, wherein the chip has a width of about 85 mm.

8. The apparatus of claim 1, wherein the chip is able to stably connect to a microplate.

9. An apparatus, comprising:
a chip comprising a predetermined reaction site having an inlet, an outlet, and a volume of less than about 1 ml, wherein the chip is constructed and arranged to be stably connectable to a microplate.

10. The apparatus of claim 9, wherein the predetermined reaction site is constructed and arranged to maintain at least one living cell at the predetermined reaction site.

11. The apparatus of claim 9, wherein the chip is enclosed.

12. The apparatus of claim 11, wherein the chip has an evaporation rate of less than about 100 microliters per day.

13. The apparatus of claim 9, wherein the chip has a length of about 128 mm.

14. The apparatus of claim 9, wherein the chip has a width of about 85 mm.

15. The apparatus of claim 9, wherein the microplate comprises at least 6 wells.

16. The apparatus of claim 15, wherein the microplate comprises at least 24 wells.

17. The apparatus of claim 16, wherein the microplate comprises at least 96 wells.

18. The apparatus of claim 17, wherein the microplate comprises at least 384 wells.

19. The apparatus of claim 18, wherein the microplate comprises at least 1,536 wells.

20. The apparatus of claim 9, wherein the microplate substantially conforms with an SBS/ANSI standard.

21. The apparatus of claim 9, wherein the chip is constructed and arranged to address at least one well of the microplate.

22. The apparatus of claim 21, wherein the chip is constructed and arranged to address more than one well of the microplate.

23. An apparatus, comprising:
a chip comprising a predetermined reaction site having an inlet, an outlet, and a volume of less than about 1 ml, wherein the chip is constructed and arranged to be fluid communicable with an apparatus constructed and arranged to address a well of a microplate.

24. The apparatus of claim 23, wherein the predetermined reaction site is constructed and arranged to maintain at least one living cell at the predetermined reaction site.

25. The apparatus of claim 23, wherein the chip is enclosed.

26. The apparatus of claim 25, wherein the chip has an evaporation rate of less than about 100 microliters per day.

27. The apparatus of claim 23, wherein the chip is constructed and arranged to address at least one well of the microplate.

28. The apparatus of claim 23, wherein the chip is constructed and arranged to address more than one well of the microplate.

29. An apparatus, comprising:
a chip comprising a predetermined reaction site having an inlet, an outlet, and a volume of less than about 1 ml, wherein each predetermined reaction site overlaps at least one well of a microplate.

30. The apparatus of claim 29, wherein the predetermined reaction site is constructed and arranged to maintain at least one living cell at the predetermined reaction site.

31. The apparatus of claim 29, wherein the chip is enclosed.

32. The apparatus of claim 31, wherein the chip has an evaporation rate of less than about 100 microliters per day.

33. The apparatus of claim 29, wherein each predetermined reaction site overlaps exactly one well of a microplate.

34. The apparatus of claim 29, wherein each predetermined reaction site overlaps more than one well of a microplate.

35. An apparatus, comprising:
a substantially liquid-tight chip comprising a predetermined reaction site having a volume of less than about 1 ml, wherein the predetermined reaction site is constructed and arranged to maintain at least one living cell at the predetermined reaction site.

36. The apparatus of claim 35, wherein the chip comprises structural components interconnected without auxiliary adhesive at locations defining boundaries of the predetermined reaction site.

37. The apparatus of claim 35, wherein the predetermined reaction site, during use of the chip, is not in fluid communication with an adhesive.

38. An apparatus, comprising:
a chip produced by a process including the step of fastening two components to produce a portion of the chip defining a predetermined reaction site having a volume of less than about 1 ml, wherein the predetermined reaction site is constructed and arranged to maintain at least one living cell at the predetermined reaction site.

39. The apparatus of claim 38, wherein the chip is enclosed.

40. The apparatus of claim 38, wherein the two components are fastened without the use of an adhesive material.

41. An apparatus, comprising:
a chip comprising a predetermined reaction site having a volume of less than about 1 ml, the predetermined reaction site constructed and arranged to maintain at least one living cell at the predetermined reaction site, wherein the predetermined reaction site has a nonzero evaporation rate of less than about 100 microliters/day.

42. The apparatus of claim 41, wherein the chip is enclosed.

43. The apparatus of claim 41, wherein the evaporation rate is less than about 50 microliters per day.

44. The apparatus of claim 43, wherein the evaporation rate is less than about 20 microliters per day.

45. In a method of producing a chip comprising a predetermined reaction site having a volume of less than 1 ml, the improvement comprising:
attaching a first component of the chip to a second component of the chip with or without auxiliary adhesive to produce a portion of the chip that defines the predetermined reaction site.

46. The method of claim 45, wherein the predetermined reaction site is constructed and arranged to maintain at least one living cell at the predetermined reaction site.

47. The method of claim 45, wherein the improvement comprises sonic welding the first component to the second component.

48. The method of claim 45, wherein the improvement comprises heat pressing the first component to the second component

49. The method of claim 45, wherein the first component comprises at least one polymer selected from the group consisting of polycarbonate, polysulfone, polyethylene, and blends and copolymers thereof.

50. The method of claim 45, wherein the improvement comprises applying energy to melt at least a portion of the first component.

51. The method of claim 50, wherein the energy comprises ultrasound.

52. The method of claim 50, wherein the energy comprises heat energy.

53. The method of claim 45, wherein the improvement comprises attaching the first component to the second component to produce a liquid-tight junction therebetween.

54. The method of claim 45, wherein the chip is enclosed.

55. An apparatus, comprising:
a predetermined reaction site having a volume of less than about 1 ml; and
a membrane substantially transparent to incident electromagnetic radiation in the infrared to ultraviolet range having a pore size less than 2.0 microns in fluid communication with the predetermined reaction site.

56. The apparatus of claim 55, wherein the predetermined reaction site is constructed and arranged to maintain at least one living cell at the predetermined reaction site.

57. An apparatus, comprising:
a predetermined reaction site having a volume of less than about 1 ml, constructed and arranged to carry out a chemical or biological reaction promoted by or monitored by electromagnetic radiation within a predetermined wavelength range; and
a membrane, transparent to electromagnetic radiation within the predetermined wavelength range to the extent necessary to promote or monitor the reaction, having a pore size of less than 2.0 microns in fluid communication with the predetermined reaction site.

58. The apparatus of claim 57, wherein the predetermined reaction site is constructed and arranged to maintain at least one living cell at the predetermined reaction site.

59. The apparatus of claim 57, wherein the membrane is substantially transparent to incident visible electromagnetic radiation.

60. The apparatus of claim 57, wherein the membrane is substantially transparent to incident electromagnetic radiation having a wavelength of between about 400 nm and about 800 nm.

61. The apparatus of claim 57, wherein the membrane has a transparency such that at least 80% of the incident electromagnetic radiation is transmitted across the membrane.

62. The apparatus of claim 61, wherein the membrane has a transparency such that at least 90% of the incident electromagnetic radiation is transmitted across the membrane.

63. The apparatus of claim 62, wherein the membrane has a transparency such that at least 95% of the incident electromagnetic radiation is transmitted across the membrane.

64. The apparatus of claim 57, wherein the membrane has an oxygen permeability of at least about 0.061 mol/day/m²/atm.

65. The apparatus of claim 57, wherein the membrane has a water permeability of less than about 0.39 mol/day/m².

66. An apparatus, comprising:
a chip comprising a first predetermined reaction site having a volume of less than about 1 ml and a second predetermined reaction site, the chip defining a pathway fluidly connecting the first predetermined reaction site and the second predetermined reaction site, wherein the pathway crosses a membrane.

67. The apparatus of claim 66, wherein the first predetermined reaction site is constructed and arranged to maintain at least one living cell at the first predetermined reaction site.

68. The apparatus of claim 66, wherein the chip is enclosed.

69. The apparatus of claim 68, wherein the chip has an evaporation rate of less than about 100 microliters per day.

70. The apparatus of claim 66, wherein the second predetermined reaction site has a volume of less than about 1 ml.

71. The apparatus of claim 66, wherein the membrane is a gas-permeable membrane.

72. The apparatus of claim 71, wherein the gas-permeable membrane is an oxygen-permeable membrane.

73. The apparatus of claim 72, wherein the oxygen-permeable membrane has an oxygen permeability of at least about 0.061 mol/day/m²/atm

74. The apparatus of claim 71, wherein the gas-permeable membrane is a CO₂-permeable membrane.

75. The apparatus of claim 66, wherein the membrane is porous.

76. The apparatus of claim 75, wherein the membrane has an average pore size of less than about 2 microns.

77. The apparatus of claim 75, wherein the membrane is substantially transparent.

78. The apparatus of claim 66, wherein the membrane is substantially transparent.

79. An apparatus, comprising:
a reaction site having a first portion and a second portion separated by a membrane; and
at least a first and a second channel in fluidic communication with the second portion of the reaction site.

80. The apparatus of claim 79, wherein the reaction site has a volume of less than 2000 microliters.

81. The apparatus of claim 79, wherein the reaction site has a volume of less than 1000 microliters.

82. The apparatus of claim 79, wherein the reaction site has a volume of less than 500 microliters.

83. The apparatus of claim 79, wherein the membrane comprises at least one of polycarbonate, cellulose, nitrocellulose, glass, fiberglass, or polycarbonate, regenerated cellulose, or polyethylene.

84. The apparatus of claim 79, wherein the membrane is permeable to cations and substantially impermeable to anions.

85. The apparatus of claim 79, wherein the membrane is permeable to anions and substantially impermeable to cations.

86. The apparatus of claim 79, wherein the membrane has a pore size less than 10 microns.

87. The apparatus of claim 79, wherein the first channel is fluidly connected to a mixing unit.

88. The apparatus of claim 87, wherein the mixing unit is fluidly connected to at least one inlet.

89. The apparatus of claim 79, wherein the substrate is formed from at least one of a glass, silicon, a metal, and a polymer.

90. The apparatus of claim 79, wherein the second portion of the reaction site is coated with a cytophilic material.

91. The apparatus of claim 79, wherein the first portion of the reaction site comprises a cytophilic material.

92. The apparatus of claim 79, further comprising a temperature sensor in sensing communication with the reaction site.

93. The apparatus of claim 79, further comprising a pH sensor in sensing communication with the reaction site.

94. The apparatus of claim 79, further comprising a pressure sensor in sensing communication with the reaction site.

95. The apparatus of claim 79, further comprising an optical density sensor in sensing communication with the reaction site.

96. The apparatus of claim 79, further comprising a glucose sensor in sensing communication with the reaction site.

97. The apparatus of claim 79, comprising at least 10 reaction sites.

98. The apparatus of claim 97, comprising at least 20 reaction sites.

99. The apparatus of claim 98, comprising at least 50 reaction sites.

100. The apparatus of claim 99, comprising at least 100 reaction sites.

101. The apparatus of claim 79, wherein the first portion is in communication with at least a third channel and a fourth channel.

102. The apparatus of claim 79, wherein the membrane is substantially impermeable to mammalian cells.

103. The apparatus of claim 79, wherein the membrane is substantially permeable to molecules having a molecular weight greater than about 100 kilodaltons.

104. The apparatus of claim 79, wherein the membrane is substantially impermeable to molecules having a molecular weight greater than about 10 kilodaltons.

105. The apparatus of claim 79, wherein the membrane is substantially impermeable to molecules having a molecular weight greater than about 1 kilodalton.

106. A method, comprising:
providing a substrate having a surface into which is fabricated a plurality of reaction sites, at least one reaction site having a volume less than about 2 ml and divided by a substantially cell impermeable membrane into at least a cell culture portion containing cells and a reservoir portion not containing cells, the reservoir portion being fluidly connected to at least a first and a second channel fabricated into the surface of the substrate;
introducing at least one test compound into at least one of the plurality of reaction sites; and
monitoring the effect of the at least one test compound on cells located within the cell culture portion.

107. The method of claim 106, wherein the membrane allows waste products produced by the cells to enter the reservoir portion.

108. The method of claim 106, wherein the membrane allows a protein produced by the cells to enter the reservoir portion.

109. The method of claim 106, wherein the contents of the reservoir portion is continuously replaced during at least a first period of time.

110. The method of claim 106, wherein the contents of the reservoir portion is periodically replaced during at least a first period of time.

111. The method of claim 106, wherein the cells include prokaryotic cells.

112. The method of claim 106, wherein the cells include eukaryotic cells.

113. The method of claim 106, wherein the membrane is a cation exchange membrane.

114. The method of claim 106, wherein the membrane is an anion exchange membrane.

115. The method of claim 106, wherein the step of monitoring comprises measuring a fluorescent signal influenced by the at least one test compound.

116. The method of claim 106, wherein the cell culture portion comprises a first type of cell and a second type of cell.
